(19) United States
(12) Patent Application Publication
(10) Pub. No.: US 2025/0127902 A1
(43) Pub. Date: Apr. 24, 2025

(54) POLYMER-CARGO COMPOSITIONS AND METHODS

(71) Applicant: REGENTS OF THE UNIVERSITY OF MINNESOTA, Minneapolis, MN (US)

(72) Inventors: Cristiam Fernando Santa Chalarca, Envigado (CO); Mckenna Grace Hanson, Denver, CO (US); Rishad Jay Dalal, San Diego, CA (US); Theresa M. Reineke, Vadnais Heights, MN (US)

(21) Appl. No.: 18/916,949

(22) Filed: Oct. 16, 2024

Related U.S. Application Data
(60) Provisional application No. 63/544,654, filed on Oct. 18, 2023.

(51) Int. Cl. A61K 47/34; A61K 9/107; A61K 31/7088; C12N 15/87

(52) U.S. Cl. CPC A61K 47/34; A61K 9/1075; A61K 31/7088; C12N 15/87

(57) ABSTRACT

A complex includes a polymer and an oligonucleotide cargo. The polymer may be a diblock copolymer that includes a first block and a second block. The first block may include an acrylate polymer. The second block may include an acrylamide polymer having one or more cationic groups, cationizable groups, or both. Alternatively, the polymer may be a homopolymer that includes one or more cationic groups, cationizable groups, or both.

### CROSS-REFERENCE TO RELATED APPLICATION

This application claims the benefit of U.S. Provisional Patent Application No. 63/544,654, filed Oct. 18, 2023, which is incorporated herein by reference in its entirety.

### SUMMARY

This disclosure describes, in one aspect a complex that includes a diblock polymer. The diblock polymer includes a first block and a second block. The first block includes an acrylate polymer. The second block includes an acrylamide polymer. The acrylamide polymer includes quaternary ammonium groups or amine cationizable groups. The complex further includes an oligonucleotide cargo.

In one or more embodiments, the acrylamide polymer is or includes amino ethyl acrylamide, dimethyl amine ethyl acrylamide, diethyl amine ethyl acrylamide, trimethyl amine ethyl acrylamide, or morpholino ethyl acrylamide.

In one or more embodiments, the acylate polymer is or includes a n-butyl acrylate polymer.

In one or more embodiments, the oligonucleotide cargo is an antisense oligonucleotide.

In one or more embodiments, the complex has an N/P ratio of 10 to 40.

In one or more embodiments, the complex includes or is a micelle.

In another aspect, the present disclosure describes a transfection composition that includes a plurality of the complexes of any preceding embodiment or aspect.

In one or more embodiments, the transfection composition includes a first plurality of complexes and a second plurality of complexes wherein the first plurality of complexes and the second plurality of complexes differ by at least by the identity of the second block of the diblock polymer.

In another aspect, the present disclosure describes a complex that includes a first diblock polymer and a second diblock polymer. The first diblock polymer includes a first block, the first block including an acrylate polymer. The first diblock polymer includes a second block, the second block including quaternary ammonium groups or amine cationizable groups. The second diblock polymer includes a first block, the first block including an acrylate polymer. The second diblock polymer includes a second block, the second block including quaternary ammonium groups or amine cationizable groups. The quaternary ammonium groups or amine cationizable groups of the second diblock polymer are different than the quaternary ammonium groups or amine cationizable groups of the first diblock polymer. The complex further includes an oligonucleotide cargo. In one or more embodiments, the complex is or includes a micelle.

In one or more embodiments, the first block of the first diblock polymer, the first block of the second diblock polymer, or both include or are an n-butyl-acrylate polymer.

In one or more embodiments, the acrylamide polymer of the first diblock polymer includes or is morpholino ethyl acrylamide and the acrylamide polymer of the second diblock polymer includes or is amino ethyl acrylamide, dimethyl amine ethyl acrylamide, diethyl amine ethyl acrylamide, or trimethyl amine ethyl acrylamide.

In one or more embodiments, the weight ratio or mole ratio of the first diblock polymer to the second diblock polymer is 0.8 parts or greater of the first diblock polymer for every 1 part of the second polymer. In one or more embodiments, the weight ratio or mole ratio of the first diblock polymer to the second diblock polymer is 0.1 to 0.3 parts of the first diblock polymer for every 1 part of the second polymer.

In another aspect, the present disclosure describes a transfection composition that includes a plurality of the complexes that include a first diblock polymer and a second diblock polymer as described herein.

In another aspect, the present disclosure describes a complex that includes a homopolymer. The homopolymer includes an acrylamide polymer. The acrylamide polymer includes quaternary ammonium groups or amine cationizable groups. The complex further includes an oligonucleotide cargo. In one or more embodiments, the oligonucleotide cargo has a length of 1000 bases or less. In one or more embodiments, the complex is or includes a polyplex. In one or more embodiments, the homopolymer is or includes amino ethyl acrylamide, dimethyl amine ethyl acrylamide, diethyl amine ethyl acrylamide, trimethyl amine ethyl acrylamide, or morpholino ethyl acrylamide.

In another aspect, the present disclosure describes a transfection composition that includes a plurality of the complexes that include a homopolymer.

In another aspect, the present disclosure describes a transfection method. The method includes contacting a cell with a transfection composition described herein.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. The synthetic scheme for cationic diblock polymers and cationic homopolymers as well as a depiction of micelle formulation. Table of properties of the cationic moieties are also shown. pKa, c log P, and molecular volume are from Santa Chalarca, C. F.; Dalal, R. J.; Chapa, A.; Hanson, M. G.; Reineke, T. M. Cation Bulk and PKa Modulate Diblock Polymer Micelle Binding to PDNA. ACS Macro Lett. 2022, 588-594. doi.org/10.1021/acsmacrolett.2c00015 where c log P and molecular volume werecalculated using the molecular property calculator of the MOLINSPIRATION CHEMINFORMATICS.

FIG. 2. GFP knockdown and cell viability in a deGFP HEK cell line with ASO complexes of polymers and micelles at an N/P 15 and 25 in simultaneous addition conditions. Trends with different cations are highlighted. Asterisks indicate the statistical significance (*: p≤0.05, **: p≤0.01) of key comparisons. Statistical analysis was done using the Brown-Forsythe and Welch ANOVA test with mean comparison using PRISM.

FIG. 3. Depiction of linear polymers settled onto adhered cells (Adhered cell addition) and comparison of GFP knockdown under simultaneous addition and adhered cell conditions for linear polyplexes.

FIG. 4. Depiction of colloidally stable micelles with cells before they have adhered during simultaneous conditions (Simultaneous Addition) and comparison of GFP knockdown under simultaneous addition and adhered cell conditions for various micelleplexes.

FIG. 5. Internalization of ASO as a function of delivery vehicle architecture (micelle, M or linear, L), N/P ratio (15 or 25), along with the controls 5 hours after transfection.

FIG. 6. Comparison of ASO internalization for simultaneous addition (solid) or adhered cell (hashed) conditions for various linear polyplexes (L).

FIG. 7. Comparison of ASO internalization using simultaneous addition (solid) or adhered cell (hashed) conditions for various micelleplexes (M).

FIG. 8. Depiction of micelles or linear polymers having different cation groups (R=AEA, DMA, DEA, TMA, or MEA) binding with an ASO to form a micelleplexe or a linear polyplexe, which are then introduced to serum.

FIG. 9. Normalized fluorescence of micelleplexes and linear polyplexes in phosphate buffered saline (PBS) indicating strength of binding. For example, comparatively, a higher normalized fluorescence value indicates weaker binding).

FIG. 10. Percent release of ASO after the addition of 10% fetal bovine serum (FBS).

FIG. 11. Dynamic light scattering data of linear polyplexes and micelleplexes including AEA or DMA at different N/Ps in the presence of 10% FBS.

FIG. 12. Dynamic light scattering data of linear polyplexes and micelleplexes including TMA or MEA at different N/Ps in the presence of 10% FBS.

FIG. 13. Relative amount of ASO in the supernatant of various linear polyplexes and micelleplexes after centrifugation.

FIG. 14. Depiction of relative amounts of linear polyplexes and micelleplexes in the supernatant and settled.

FIG. 15. Synthesis scheme for aminolysis and Michael addition of a linear poly(pentafluorophenyl acrylate) PFPA polymer to install a cation group (TMA, AEA, DEA, DMA, MEA).

FIG. 16. ¹⁹F NMR of crude products showing complete aminolysis of PFPA polymer yielding pentafluorophenol.

FIG. 17. ¹H NMR of L-TMA in DMF D7:D₂O 3:1.

FIG. 18. ¹H NMR of L-AEA-Boc in DMF D7.

FIG. 19. ¹H NMR of L-AEA in DMF D7:D₂O 3:1.

FIG. 20. ¹H NMR of L-DEA in D₂O.

FIG. 21. ¹H NMR of L-DMA in dimethylformamide (DMF) D7.

FIG. 22. ¹H NMR of L-MEA in D₂O.

FIG. 23. Size exclusion chromatography (SEC) characterization data of linear polymers.

FIG. 24. SEC traces of linear polymers.

FIG. 25. Dynamic light scattering (DLS) traces of micelles that include a polymer having TMA groups (top) or DEA groups (bottom).

FIG. 26. DLS traces of micelles that include a polymer having DMA groups (top), AEA groups (middle) or MEA groups (bottom).

FIG. 27. GFP knockdown after 72 hours of various linear polyplexes and micelleplexes formulations.

FIG. 28. Cell viability after 72 hours of various linear polyplexes and micelleplexes formulations.

FIG. 29. GFP knockdown after 72 hours of various linear polyplexes and micelleplexes with a mismatch (MM) ASO. (“A” is adhered cell addition and “S” is simultaneous addition).

FIG. 30. Percent of cells with Cy3 fluorescence 5 hours after transfection with a Cy3-ASO and corresponding delivery vehicle (e.g., linear polyplex or micelleplex). (“A” is adhered cell addition and “S” is simultaneous addition).

FIG. 31. Percent of cells with Cy3 fluorescence 24 hours after transfection with a Cy3-ASO and corresponding delivery vehicle (e.g., linear polyplex or micelleplex). (“A” is adhered cell addition and “S” is simultaneous addition).

FIG. 32. Relative amount of Cy3 fluorescence 24 hours after transfection with a Cy3-ASO and corresponding delivery vehicle (e.g., linear polyplex or micelleplex). (“A” is adhered cell addition and “S” is simultaneous addition).

FIG. 33. Data showing which condition (complexes added to adhered cells (“A”) or complexes added simultaneously with cells (“S”)) had higher knockdown or Cy3 internalization (comparing mean fluorescence). Bold indicates opposite trends as predicted with the matching colloidal stability hypothesis. An unpaired parametric t test was used to compare each condition. Asterisks indicate statistical significance (ns: p>0.05, *: p≤0.05, **: p≤0.01, ***: p≤0.001, ****: p≤0.0001). The ASO internalization after 24 hours is highest under simultaneous addition conditions for both micelles and linear polymers.

FIG. 34. DLS traces of micelles, linear polymers, micelleplexes, and polyplexes without serum. The micelles, linear polymers, micelleplexes, and polyplexes included DEA or DMA.

FIG. 35. DLS traces of micelles, linear polymers, micelleplexes, and polyplexes without serum. The micelles, linear polymers, micelleplexes, and polyplexes included MEA, AEA, or TMA.

FIG. 36. Cationic moieties (R groups) in each diblock polymer. Properties of the cationic moieties. pKa is from Santa Chalarca, C. F.; Dalal, R. J.; Chapa, A.; Hanson, M. G.; Reineke, T. M. Cation Bulk and PKa Modulate Diblock Polymer Micelle Binding to PDNA. ACS Macro Lett. 2022, 588-594. doi.org/10.1021/acsmacrolett.2c00015. c log P was calculated using the molecular property calculator of the MOLINSPIRATION CHEMINFORMATICS.

FIG. 37. Types of micelles formulated and their nomenclature.

FIG. 38. DLS traces for homomicelles A100, D100, and M100 and blended diblocks BldA50M50 and BldD50M50.

FIG. 39. DLS traces for micelleplexes in PBS at designated N/P of ASO and homomicelles A100, D100, and M100 and blended diblocks BldA50M50 and BldD50M50, and mixed micelles, MixA50+M50 and MixD50+M50.

FIG. 40. Binding strength in PBS at designated N/P of ASO and homomicelles A100, D100, and M100; blended diblocks BldA50M50 and BldD50M50; and mixed micelles MixA50+M50 and MixD50+M50.

FIG. 41. Illustration of the transfection procedure where cells were temporarily suspended in solution and combined with micelle formulations.

FIG. 42. The formulations/cells were then added to the plate for transfection and a GFP knockdown assay was performed. GFP knockdown performance 72 hours after transfection with each micelleplex formulation. Note that since ASO only GFP knockdown is approximately 0 within error with and without BAF-A1, ASO only is not plotted. Asterisks indicate the statistical significance (*: p≤0.05, ns: p>0.05) of key comparisons. Statistical analysis was done using the Brown-Forsythe and Welch ANOVA test with mean comparison.

FIG. 43. Cell Viability 72 hours after transfection with each micelleplex formulation. Note that since ASO only GFP knockdown is approximately 0 within error with and without BAF-A1, ASO only is not plotted. Asterisks indicate the statistical significance (*: p≤0.05, ns: p>0.05) of key comparisons. Statistical analysis was done using the Brown-Forsythe and Welch ANOVA test with mean comparison.

FIG. 44. Percent decrease in GFP knockdown 72 hours after transfection when cells were preincubated with BAF-A1 compared to without preincubation with BAF-A1. Note that since ASO only GFP knockdown is approximately 0 within error with and without BAF-A1, ASO only is not plotted. Asterisks indicate the statistical significance (*: p≤0.05, ns: p>0.05) of key comparisons. Statistical analysis was done using the Brown-Forsythe and Welch ANOVA test with mean comparison.

FIG. 45. DLS traces for micelleplexes in PBS at designated N/P of ASO and homomicelles (D100 and M100), blended diblocks (BldD20M80, BldD50M50, BldD80M20), and mixed micelles (MixD20+M80, MixD50+M50, and MixD80+M20).

FIG. 46. Binding strength in PBS at designated N/P of ASO and homomicelles (D100 and M100), blended diblocks (BldD20M80, BldD50M50, BldD80M20), and mixed micelles (MixD20+M80, MixD50+M50, and MixD80+M20). Binding strength after 2 times volume addition of DMEM with 10% FBS at designated N/P of ASO and homomicelles (D100 and M100), blended diblocks (BldD20M80, BldD50M50, BldD80M20), and mixed micelles (MixD20+M80, MixD50+M50, and MixD80+M20).

FIG. 47. GFP knockdown 72 hours after transfection with each micelleplex formulation of D and M at different ratios. Asterisks indicate the statistical significance (*: p≤0.05, **: p≤0.01, ***: p≤0.001, ns: p>0.05) of key comparisons. Statistical analysis was done using the Brown-Forsythe and Welch ANOVA test with mean comparison using Prism.

FIG. 48. Cell viability 72 hours after transfection with each micelleplex formulation of D and M at different ratios. Asterisks indicate the statistical significance (*: p≤0.05, **: p≤0.01, ***: p≤0.001, ns: p>0.05) of key comparisons. Statistical analysis was done using the Brown-Forsythe and Welch ANOVA test with mean comparison using Prism.

FIG. 49. Multiple of cell viability and GFP knockdown, designated as effective efficiency, 72 hours after transfection with each micelleplex formulation of D and M at different ratios. Asterisks indicate the statistical significance (*: p≤0.05, **: p≤0.01, ***: p≤0.001, ns: p>0.05) of key comparisons. Statistical analysis was done using the Brown-Forsythe and Welch ANOVA test with mean comparison using Prism.

FIG. 50. Percent decrease in GFP knockdown 72 hours after transfection when cells were preincubated with BAF-A1 compared to without preincubation with BAF-A1. Asterisks indicate the statistical significance (*: p≤0.05, **: p≤0.01, ***: p≤0.001, ns: p>0.05) of key comparisons. Statistical analysis was done using the Brown-Forsythe and Welch ANOVA test with mean comparison using Prism.

FIG. 51. Effective efficiency for mixed micelles and diblocks at 50:50 ratios.

FIG. 52. Raw GFP knockdown data for BAF-A1 transfection compared to GFP knockdown without BAF-A1 transfection.

FIG. 53. Binding of an ASO to micelles in DMEM and 10% FBS.

FIG. 54. GFP knockdown 72 hours after transfection with each micelleplex formulation and the designated amount of chloroacetate (CA).

FIG. 55. Cell viability 72 hours after transfection with each micelleplex formulation.

FIG. 56. Mismatched GFP Knockdown 72 hours after transfection with each micelleplex formulation.

FIG. 57. Multiple of cell viability and GFP knockdown, designated as effective efficiency, 72 hours after transfection with each micelleplex formation.

FIG. 58. GFP knockdown data for blended and mixed micelles of different ratios of M and A. Note that these micelles were formulated in chloroacetate buffer (20 mM, pH=3.0 100 ionic strength adjusted with NaCl) and therefore aren't directly comparable to other data.

FIG. 59. Raw GFP knockdown data for BAF-A1 transfection compared to GFP knockdown without BAF-A1 transfection.

### DETAILED DESCRIPTION OF ILLUSTRATIVE EMBODIMENTS

Nucleic acid-based medicines have many important applications ranging from disease treatment to vaccine development. Antisense oligonucleotides (ASOs) are one type of pharmaceutical paradigm emerging as an innovative medicine for many diseases including cancer and spinal muscular atrophy. Some ASOs function by binding to RNA, such as mRNA, via Watson-Crick base pairing to modulate gene expression. For example, binding of an ASO to RNA can induce one or more mechanisms to prevent the translation of RNA into a protein, induce ribonuclease H mediated decay of RNA, and/or modulate the splicing of pre-mRNA. ASOs were one of the first genetic therapeutics on the market because new ASOs can be readily developed with knowledge of the targeted mRNA sequence. For example, MILASEN is an ASO therapy developed specifically for one patient suffering from Batten disease, a fatal genetic disease-causing loss of vision, seizures, and dementia. The patient's genome was sequenced to identify the exact mutation thought to cause the disease, and a specific ASO was developed (MILASEN) that improved the patient's health.

Although ASOs have the potential to change the way diseases are treated, the physiological environment makes ASO delivery challenging. To engage a target, the ASO may need to cross several cellular barriers including the cell membrane and the endosome. Due to the relatively large and anionic nature of many ASOs, crossing cellular barriers is challenging. Additionally, for in vivo applications, the ASO should avoid extracellular barriers such as nuclease degradation, renal clearance, protein sequestration, among others, to even arrive at the cellular barriers.

Delivery vehicles are commonly used to facilitate delivery of genetic payloads such as ASOs through the cell membrane. Delivery vehicles include viral vectors and lipid-based systems. Viral vectors are expensive and a potentially immunogenic gene delivery system. While more economically tractable than viral vector systems, lipid-base systems face stability issues.

Polymer-based delivery systems are being explored as genetic payload delivery systems. Polymer-based delivery systems may have improved stability over lipid-based delivery systems because polymers are large molecules that are less dynamic, whereas lipid-based systems are complex and involve dynamic assemblies of many lipids with semi-stable structures. Cationic polymers bind to anionic genetic material to form complexes such as polyplexes, which can facilitate delivery of many nucleic acid types (e.g., ASOs). Synthesis of cationic polymers is modular, easy to scale up, and inexpensive. Cationic polymers bind to oligonucleotides, such as ASOs, through interpolyelectrolyte complexation driven by entropy, forming complexes. While many cationic polymer structures and architectures have been studied, cationic polymers still have issues translating to clinical use. For example, serum instability of formulations is a challenge for nonviral formulations. Serum, a component of blood that includes many types of proteins, can cause aggregation, can cause burst release of payloads, and/or can bind nonspecifically to delivery systems, causing issues with both in vitro and in vivo delivery. Methods of improving serum stability include the incorporation of hydrophilic sheath layers such as polyethylene glycol, carbohydrates, or zwitterionic moieties. However, these methods of improving serum stability can decrease interactions of the cationic polymer with the oligonucleotide payload and/or the interactions between the complex and a cell, both of which can result in decreased efficacy and/or efficiency of such cationic polymer delivery systems.

In one aspect, the present disclosure describes a complex that includes a polymer and an oligonucleotide cargo. The polymer includes cationic groups, cationizable groups, or both. In another aspect, the present disclosure describes a composition that includes one or more of the complexes of the present disclosure. In yet another aspect, the present disclosure describes methods of using a complex and/or a composition containing the same. The complex and/or composition containing the same may be used as or in delivery systems for oligonucleotide cargo. The delivery system may be used for in vitro and/or in vivo delivery of an oligonucleotide cargo.

The complexes include an oligonucleotide cargo. As used herein, the terms “oligonucleotide” and “nucleic acid” are used interchangeably to refer to a polymer of two or more nucleotides. As use herein, the term “oligonucleotide cargo” refers to one or more oligonucleotides that are a part of a complex of the present disclosure. An oligonucleotide cargo may include a plurality of oligonucleotides. The plurality of oligonucleotides may include the oligonucleotide having the same sequence or two or more oligonucleotides of different sequences.

An oligonucleotide of an oligonucleotide cargo may be single stranded; double stranded; or include one or more portions that are single stranded and one or more portions that are double stranded. Oligonucleotides include nucleosides linked through internucleoside linkages, also called backbone linkages. Nucleosides include a pentose sugar (e.g., ribose or deoxyribose) and a nitrogenous base (nucleobase or base) covalently attached to the sugar. The canonical nucleobases found in DNA and/or RNA are adenine (A), guanine (G), thymine (T), cytosine (C), and uracil (U). The canonical sugars found in DNA and/or RNA are deoxyribose (DNA) and ribose (RNA). A conical nucleoside linkage (also termed backbone linkage) is a phosphodiester bond. Oligonucleotide cargos may include canonical sugars, canonical bases, canonical internucleoside linkages, non-canonical sugars, non-canonical bases, non-canonical internucleoside linkages, or any combination thereof. Examples of non-canonical sugars include 2′ modifications on a pentose or ribose such as 2′-O-methoxyethyl (2′O-MOE) and 2′ methoxy (2′O-Me); locked sugars (also known as locked nucleic acids); and sugar alternatives such as methylenemorpholine rings. Examples of noncanonical backbone linkages include phosphorothioate linkages and phosphorodiamidate linkages. In some cases, a non-canonical sugar can be combined with a non-canonical backbone linkage, such as, for example in a phosphorodiamidate morpholino oligomer.

Examples of oligonucleotide cargos include, but are not limited to, plasmid DNA (pDNA), messenger RNA (mRNA), antisense oligonucleotides (ASOs), small interfering RNA (siRNA), micro-RNA (miRNA), guide RNA (gRNA), Cas9-gRNa complexes, aptamers, derivatives thereof, and combinations thereof.

In one or more embodiments, the oligonucleotide cargo includes an antisense oligonucleotide (ASO). An ASO is a single stranded oligonucleotide that is at least partially complementary to a target nucleic acid. ASOs are generally single stranded oligonucleotides; however, in the complexes of the present disclosure, an ASO may exists as single-stranded oligonucleotide, a double stranded oligonucleotide, or have one or more single stranded regions and one or more double stranded regions. ASOs may be linear and lack secondary structure. ASOs may include one or more non-canonical bases, one or more non-canonical sugars, one or more non-canonical internucleoside linkages, or any combination thereof.

An oligonucleotide cargo may have a variety of lengths. In one or more embodiments, the number of bases in an oligonucleotide cargo is 2 or more, 5 or more, 10 or more, 15 or more, 20 or more, 25 or more, 30 or more, 35 or more, 40 or more, 50 or more, 75 or more, 100 or more, 200 or more, 300 or more, 400 or more, 500 or more, 600 or more, 700 or more, 800 or more, 900 or more, 1000 or more, 10000 or more, 20000 or more, 30000 or more, or 40000 or more. In one or more embodiments, the number of bases in an oligonucleotide cargo is 50000 or less, 40000 or less, 30000 or less, 20000 or less, 10000 or less, 1000 or less, 900 or less, 800 or less, 700 or less, 600 or less, 500 or less, 400 or less, 300 or less, 200 or less, 100 or less, 75 or less, 50 or less, 40 or less, 35 or less, 30 or less, 25 or less, 20 or less, 15 or less, 10 or less, or 5 or less.

In one or more embodiments, the number of bases in an oligonucleotide cargo is 5 to 1000. In one or more embodiments, the oligonucleotide cargo is an ASO and the number of bases in the ASO is 5 to 1000, 5 to 75, 5 to 50, 5 to 30, 10 to 50, 10 to 40, 10 to 13, 15 to 50, 15 to 40, 15 to 30, 15 to 25, 20 to 50, 20 to 40, or 20 to 30.

In one or more embodiments, the oligonucleotide cargo is not self-replicating. In one or more embodiments, the oligonucleotide cargo is not methylated; that is, any cytosine present in the oligonucleotide sequence is not a methylated cytosine. In one or more embodiments, the oligonucleotide cargo does not encode a gene product.

The sequence of an oligonucleotide cargo depends at least in part upon the target nucleic acid. Targeting an oligonucleotide cargo to a target nucleic acid may be a multistep process. The process may include identifying the nucleic acid of interest, analyzing a transcript of the nucleic acid of interest, and identifying a particular region of the nucleic acid of interest to be targeted by the oligonucleotide. The nucleic acid of interest may be a pre-mRNA transcript, mRNA transcript, or a gene associated with a disease or disorder. Methods for designing, synthesizing, and screening oligonucleotide cargos are known.

A complex of the present disclosure includes a polymer. Polymers are polymerized from one or more monomers. A polymer polymerized from a particular monomer may be described as “monomer name” polymer. For example, a polymer polymerized from n-butyl acrylate monomers, may be described as an n-butyl acrylate polymer.

In one or more embodiments, a complex of the present disclosure includes a homopolymer. A homopolymer is a polymer polymerized from a single monomer.

In one or more embodiment, a complex of the present disclosure includes a block polymer. A block polymer is a polymer that includes two or more polymer segments (blocks) joined by a covalent linkage. A block copolymer may include two or more homopolymer segments; two or more copolymer segments; or one or more homopolymer segments and one or more copolymer segments that are joined by a covalent linkage.

In one or more embodiments, a complex includes a diblock polymer. A diblock polymer is a polymer that includes two homopolymer segments joined by a covalent linkage.

The homopolymers and block polymers of the present disclosure include cationic groups (also called cation groups, cationic moieties, cation moiety, or simply cations) or cationizable groups. A cationic group is a chemical functionality that has a positive charge. A cationizable group is a chemical functionality that when exposed to some pH values is protonated resulting in a positive charge. In some embodiments of the present disclosure, a cationizable group is an amine cationizable group that can be protonated to from a cation. In some embodiments of the present disclosure, a cationizable group is an amine cationizable group that has a pKa of 8.5 or less, 8.0 or less, 7.5 or less, 7.0 or less, 6.5 or less, 6.0 or less, or 5.5 or less. For example, an amine cationizable group may be protonated at physiological pH (e.g., 7.0-7.4) and/or at an acid pH, such as the pH observed in an endosome (e.g., 3 to 6.5). The cationic groups or cationizable groups may be a part of a monomer used to form a polymer. For example, a monomer may include a pendant cationic group or pendent cationizable group that is not chemically modified during polymerization.

In one or more embodiments, a cationic group includes a quaternary ammonium. In one or more embodiments a cationizable group is an amine cationizable group. An amine cationizable group includes a primary amine, a secondary amine, or a tertiary amine. Under certain conditions, an amine can be protonated forming an ammonium cation. For example, a primary amine can be protonated forming a primary ammonium cation, a secondary amine can be protonated forming a secondary ammonium cation, and a tertiary amine can be protonated forming a tertiary ammonium cation. In one or more embodiments, the cationic group includes an amine that is a part of a ring.

The cationizable groups of a polymer of the present disclosure may be described in their neutral form or in their cationic group form.

A cationic group or a cationizable group may be of the formula —NR¹R²R³ where R¹, R², and R³ are each independently H, a lone pair of electrons, or alkyl. The alkyl may include 1, 2, 3, 4, 5, 6, 7, 8, 9, or 10 carbons. In one or more embodiments, the alkyl is methyl, ethyl, isopropyl, n-propyl, iso-butyl, n-butyl, or sec-butyl. In one or more embodiments, R¹, R², and R³ are the same. In one or more embodiments, R¹ and R² are the same and R³ is different. In one or more embodiments, R¹, R², and R³ are different. In embodiments where R¹, R², and R³ are all alkyl, the group is a cationic group. An example of such a cationic group is trimethylammonium (—N(CH₃)₃⁺). Examples of cationizable groups include —NH₂; dimethylamino (—N(CH₃)₂); and diethylamino (—N(CH₂CH₃)₂). When exposed to certain pH values, the dimethylamino and diethylamino groups can be protonated to form dimethylammonium (—NH(CH₃)₂⁺) and diethylammonium (—NH(CH₂CH₃)₂⁺) groups, respectively.

A cationizable group may be of the formula —NR⁴R⁵ where the nitrogen, R⁴ and R⁵ form a 5, 6, or 7 membered ring. Except for the nitrogen atom, the ring may be a hydrocarbon ring. The ring may be aromatic. In addition to the nitrogen atom of the formula, the ring may include one or more other heteroatoms such as O, N, or S. Examples of cationic groups that are rings include 1-imidazolyl and 2-morpholino.

The acid dissociation constant (pKa) of a polymer that includes cationic groups or cationizable may vary. The pKa of a polymer may affect the ability of the polymer to form complexes with an oligonucleotide cargo and/or the ability to release the oligonucleotide cargo from a complex. The pKa of a polymer may be determined, for example, via potentiometric titration. In one or more embodiments, the pKa of the polymer is 5.0 or greater, 5.5 or greater, 6.0 or greater, 6.1 or greater, 6.2 or greater, 6.3 or greater, 6.4 or greater, 6.5 or greater, 6.6 or greater, 6.7 or greater, 6.8 or greater, 6.9 or greater, 7.0 or greater, 7.2 or greater, or 7.5 or greater. In one or more embodiments, the pKa of the polymer is 8.0 or less, 7.5 or less, 7.2 or less, 7.0 or less, 6.9 or less, 6.8 or less, 6.7 or less, 6.6 or less, 6.5 or less, 6.4 or less, 6.3 or less, 6.2 or less, 6.1 or less, 6.0 or less, or 5.5 or less. In one or more embodiments the pKa of the polymer is 5.0 to 8.0, 5.0 to 7.5, 5.0 to 7.2, 5.0 to 7.0, 5.0 to 6.9, 5.0 to 6.8, 5.0 to 6.7, 5.0 to 6.6, 5.0 to 6.5, 5.0 to 6.4, 5.0 to 6.3, 5.0 to 6.2, 5.0 to 6.1, 5.0 to 6.0, or 5.0 to 5.5. In one or more embodiments the pKa of the polymer is 5.5 to 8.0, 5.5 to 7.5, 5.5 to 7.2, 5.5 to 7.0, 5.5 to 6.9, 5.5 to 6.8, 5.5 to 6.7, 5.5 to 6.6, 5.5 to 6.5, 5.5 to 6.4, 5.5 to 6.3, 5.5 to 6.2, 5.5 to 6.1, or 5.5 to 6.0. In one or more embodiments the pKa of the polymer is 6.0 to 8.0, 6.0 to 7.5, 6.0 to 7.2, 6.0 to 7.0, 6.0 to 6.9, 6.0 to 6.8, 6.0 to 6.7, 6.0 to 6.6, 6.0 to 6.5, 6.0 to 6.4, 6.0 to 6.3, 6.0 to 6.2, or 6.0 to 6.1. In one or more embodiments the pKa of the polymer is 6.1 to 8.0, 6.1 to 7.5, 6.1 to 7.2, 6.1 to 7.0, 6.1 to 6.9, 6.1 to 6.8, 6.1 to 6.7, 6.1 to 6.6, 6.1 to 6.5, 6.1 to 6.4, 6.1 to 6.3, or 6.1 to 6.2. In one or more embodiments the pKa of the polymer is 6.2 to 8.0, 6.2 to 7.5, 6.2 to 7.2, 6.2 to 7.0, 6.2 to 6.9, 6.2 to 6.8, 6.2 to 6.7, 6.2 to 6.6, 6.2 to 6.5, 6.2 to 6.4, or 6.2 to 6.3. In one or more embodiments the pKa of the polymer is 6.3 to 8.0, 6.3 to 7.5, 6.3 to 7.2, 6.3 to 7.0, 6.3 to 6.9, 6.3 to 6.8, 6.3 to 6.7, 6.3 to 6.6, 6.3 to 6.5, or 6.3 to 6.4. In one or more embodiments the pKa of the polymer is 6.4 to 8.0, 6.4 to 7.5, 6.4 to 7.2, 6.4 to 7.0, 6.4 to 6.9, 6.4 to 6.8, 6.4 to 6.7, 6.4 to 6.6, or 6.4 to 6.5. In one or more embodiments the pKa of the polymer is 6.5 to 8.0, 6.5 to 7.5, 6.5 to 7.2, 6.5 to 7.0, 6.5 to 6.9, 6.5 to 6.8, 6.5 to 6.7, or 6.5 to 6.6. In one or more embodiments the pKa of the polymer is 6.6 to 8.0, 6.6 to 7.5, 6.6 to 7.2, 6.6 to 7.0, 6.6 to 6.9, 6.6 to 6.8, or 6.6 to 6.7. In one or more embodiments the pKa of the polymer is 6.7 to 8.0, 6.7 to 7.5, 6.7 to 7.2, 6.7 to 7.0, 6.7 to 6.9, or 6.7 to 6.8. In one or more embodiments the pKa of the polymer is 6.8 to 8.0, 6.8 to 7.5, 6.8 to 7.2, 6.8 to 7.0, or 6.8 to 6.9. In one or more embodiments the pKa of the polymer is 6.9 to 8.0, 6.9 to 7.5, 6.9 to 7.2, or 6.9 to 7.0. In one or more embodiments the pKa of the polymer is 7.0 to 8.0, 7.0 to 7.5, or 7.0 to 7.2. In one or more embodiments the pKa of the polymer is 7.2 to 8.0, 7.2 to 7.5, or 7.5 to 8.0.

In one or more embodiments, the complex includes an acrylamide polymer, the acrylamide polymer including the cationic groups and/or the cationizable groups. An acrylamide polymer includes the repeating acrylamide functionality —CH₂CH(C(═O)NHR)— where R includes a cationic/cationizable group. R can include a linker that separates the cationic/cationizable group from the amide of the acrylamide functionality. The linker can be a hydrocarbon linker. The hydrocarbon linker can include —(CH₂)ₙ— where n is 1, 2, 3, 4, 5, 6, 7, 8, 9, or 10. Examples of acrylamide polymers include, but are not limited to, amino ethyl acrylamide (AEA; AEAm); dimethyl amine ethyl acrylamide (DMA; DMAm); diethyl amine ethyl acrylamide (DEA; DEAm); trimethyl amine ethyl acrylamide (TMA; TMAm); morpholino ethyl acrylamide (MEA; MEAm); trimethylammonium ethylamine acrylamide (TMAEA; TMAEAm); and (1-imidazolyl)propyl acrylamide (ImPAm).

An acrylamide polymer can be formed from acrylamide monomers. An acrylamide polymer can be formed by reacting a polymer having a carboxylic acid or an ester with a compound that includes an amine capable of forming an amide bond. For example, an acrylamide polymer can be formed by modifying an acrylate polymer. Acrylate monomers may be polymerized to form an acrylate polymer having the acrylate functionality repeating group —CH₂CH(C(═O)OR^{X})— where R^{X} is a labile ester functionality. A compound having an amine capable of reacting with the ester of the acrylate functionality can react with the ester to displace RX and form an amide bond thereby forming an acrylamide functionality. An acrylamide polymer formed this way may include residual acrylate functionalities that were not converted to acrylamide functionalities; that is, the polymer may include residual —CH₂CH(C(═O)OR^{X})— groups. As used herein, the term “acrylamide polymer” refers to a polymer or polymer segment (block) that includes 80 mole percent (mol-%) or greater, 85 mol-% or greater, 90 mol-% or greater, 95 mol-% or greater, or 99 mol-% or greater acrylamide functionalities.

In one or more embodiments, a complex of the present disclosure includes a diblock polymer. A diblock polymer includes a first block and a second block. The first block includes a first homopolymer and the second block includes a second homopolymer. The first homopolymer may be a homopolymer that includes cationic groups or cationizable groups. In one or more embodiments, the first homopolymer (first block) includes an acrylamide polymer where the acrylamide polymer includes cationic groups or cationizable groups.

The second homopolymer (second block) of a diblock polymer may be a polymer that allows for the formation of micelles that include the diblock polymer (described herein). The second homopolymer may include a hydrophobic polymer. In one or more embodiments, the second homopolymer includes a hydrophobic group. The hydrophobic group may be alkyl. The alkyl may be linear, branched, or cyclic. The alkyl may include 3, 4, 5, 6, 7, 8, 9, or 10 carbon atoms. Examples of hydrophobic alkyl groups include isopropyl, n-propyl, iso-butyl, n-butyl, and sec-butyl.

The second homopolymer may be an acrylate polymer or an acrylamide polymer. As such, the second homopolymer may include the repeating acrylamide functionality (—CH₂CH(C(═O)NHR^{Y})—) or the repeating acrylate functionality (—CH₂CH(C(═O)OR^{Y})—) where R^{Y} the is a hydrophobic group described herein. Examples of acrylate polymers include isopropyl acrylate (poly; n-butyl acrylate; sec-butyl; and combinations thereof acrylate polymers. Examples of acrylate polymers include isopropyl acrylamide; n-butyl acrylamide; sec-butyl; and combination thereof acrylamide polymers.

In embodiments where the complex includes a diblock polymer the diblock polymer may be of the formula [block 1]_{z}-[block 2]_{q} were z an q are each independently an integer from 1 to 1000.

The number-average molecular weight (Mn) of a polymer of a complex of the present disclosure may vary. Mn is calculated using the following equation:

$$Mn = \sum {x}_{i} {M}_{i}$$

where Mᵢ is the mean molecular size of range i and xᵢ is the number fraction of the total number of polymer chains that are within Mᵢ range. Mn may be determined, for example, using size exclusion chromatography with a multi-angle light scattering detector.

In one or more embodiments, the Mn of the polymer is 5 kilodalton (kDa) or greater, 10 kDa or greater, 20 kDa or greater, 25 kDa or greater, 30 kDa or greater, 40 kDa or greater, 50 kDa or greater, or 75 kDa or greater. In one or more embodiments, the Mn of the polymer is 100 kDa or less, 75 kDa or less, 50 kDa or less, 40 kDa or less, 30 kDa or less, 25 kDa or less, 20 kDa or less, or 10 kDa or less. In one or more embodiments, the Mn of the polymer is 5 kDa to 100 kDa, 5 kDa to 75 kDa, 5 kDa to 50 kDa, 5 kDa to 40 kDa, 5 kDa to 30 kDa, 5 kDa to 25 kDa, 5 kDa to 20 kDa, or 5 kDa to 10 kDa. In one or more embodiments, the Mn of the polymer is 10 kDa to 100 kDa, 10 kDa to 75 kDa, 10 kDa to 50 kDa, 10 kDa to 40 kDa, 10 kDa to 30 kDa, 10 kDa to 25 kDa, or 10 kDa to 20 kDa. In one or more embodiments, the Mn of the polymer is 20 kDa to 100 kDa, 20 kDa to 75 kDa, 20 kDa to 50 kDa, 20 kDa to 40 kDa, 20 kDa to 30 kDa, or 20 kDa to 25 kDa. In one or more embodiments, the Mn of the polymer is 25 kDa to 100 kDa, 25 kDa to 75 kDa, 25 kDa to 50 kDa, 25 kDa to 40 kDa, or 25 kDa to 30 kDa. In one or more embodiments, the Mn of the polymer is 30 kDa to 100 kDa, 30 kDa to 75 kDa, 30 kDa to 50 kDa, or 30 kDa to 40 kDa. In one or more embodiments, the Mn of the polymer is 40 kDa to 100 kDa, 40 kDa to 75 kDa, or 40 kDa to 50 kDa. In one or more embodiments, the Mn of the polymer is 50 kDa to 100 kDa or 50 kDa to 75 kDa. In one or more embodiments, the Mn of the polymer is 75 kDa to 100 kDa.

The weight-average molecular weight (Mw) of a polymer of the present disclosure may vary. Mw is calculated using the following equation:

$$Mw = \sum {w}_{i} {M}_{i}$$

where Mᵢ is the mean molecular size of range i and wᵢ is the weight fraction of the total number of polymer chains that are within Mᵢ range. Mw may be determined using size exclusion chromatography with a multi-angle light scattering detector (see Example).

In one or more embodiments, the Mw of the polymer is 5 kilodalton (kDa) or greater, 10 kDa or greater, 20 kDa or greater, 25 kDa or greater, 30 kDa or greater, 40 kDa or greater, 50 kDa or greater, or 75 kDa or greater. In one or more embodiments, the Mw of the polymer is 100 kDa or less, 75 kDa or less, 50 kDa or less, 40 kDa or less, 30 kDa or less, 25 kDa or less, 20 kDa or less, or 10 kDa or less. In one or more embodiments, the Mw of the polymer is 5 kDa to 100 kDa, 5 kDa to 75 kDa, 5 kDa to 50 kDa, 5 kDa to 40 kDa, 5 kDa to 30 kDa, 5 kDa to 25 kDa, 5 kDa to 20 kDa, or 5 kDa to 10 kDa. In one or more embodiments, the Mw of the polymer is 10 kDa to 100 kDa, 10 kDa to 75 kDa, 10 kDa to 50 kDa, 10 kDa to 40 kDa, 10 kDa to 30 kDa, 10 kDa to 25 kDa, or 10 kDa to 20 kDa. In one or more embodiments, the Mw of the polymer is 20 kDa to 100 kDa, 20 kDa to 75 kDa, 20 kDa to 50 kDa, 20 kDa to 40 kDa, 20 kDa to 30 kDa, or 20 kDa to 25 kDa. In one or more embodiments, the Mw of the polymer is 25 kDa to 100 kDa, 25 kDa to 75 kDa, 25 kDa to 50 kDa, 25 kDa to 40 kDa, or 25 kDa to 30 kDa. In one or more embodiments, the Mw of the polymer is 30 kDa to 100 kDa, 30 kDa to 75 kDa, 30 kDa to 50 kDa, or 30 kDa to 40 kDa. In one or more embodiments, the Mw of the polymer is 40 kDa to 100 kDa, 40 kDa to 75 kDa, or 40 kDa to 50 kDa. In one or more embodiments, the Mw of the polymer is 50 kDa to 100 kDa or 50 kDa to 75 kDa. In one or more embodiments, the Mw of the polymer is 75 kDa to 100 kDa.

The dispersity of the molecular weight of the polymer affect the characteristics of the polymer. The molecular weight dispersity may be quantified as the dispersity (Ð_{M}). Ð_{M} is the distribution of individual molecular masses of a polymer. Ð_{M} is calculated as the quotient of the mass average molecular weight (Mw) divided by the number-average molecular weight (Mn). The Mw and Mn may be determined using various methods including, for example, viscometry, size exclusion chromatography, and mass spectrometry. Generally, a small Ð_{M} is preferred. Although there is no desired lower limit, in practice the Ð_{M} of the polymer may be 1.0 or greater, 1.1 or greater, 1.2 or greater, 1.3 or greater, 1.4 or greater, 1.5 or greater, 1.6 or greater, 1.7 or greater, 1.8 or greater, 1.9 or greater, 2.0 or greater, 2.1 or greater, 2.2 or greater, 2.3 or greater. 2.3 or greater, or 2.4 or greater. In one or more embodiments, the Ð_{M} of the polymer may be 2.5 or less, 2.2 or less, 2.0 or less, 1.8 or less, 1.7 or less, 1.6 or less, 1.5 or less, 1.4 or less, 1.3 or less, 1.2 or less, or 1.1 or less. In one or more embodiments, the Ð_{M} for the polymer is 1.0 to 2.5, 1.0 to 2.2, 1.0 to 2.0, 1.0 to 1.8, 1.0 to 1.7, 1.0 to 1.6, 1.0 to 1.5, 1.0 to 1.4, 1.0 to 1.3, 1.0 to 1.2, or 1.0 to 1.1. In one or more embodiments, the Ð_{M} for the polymer is 1.1 to 2.5, 1.1 to 2.2, 1.1 to 2.0, 1.1 to 1.8, 1.1 to 1.7, 1.1 to 1.6, 1.1 to 1.5, 1.1 to 1.4, 1.1 to 1.3, or 1.1 to 1.2. In one or more embodiments, the Ð_{M} for the polymer is 1.2 to 2.5, 1.2 to 2.2, 1.2 to 2.0, 1.2 to 1.8, 1.2 to 1.7, 1.2 to 1.6, 1.2 to 1.5, 1.2 to 1.4, or 1.2 to 1.3. In one or more embodiments, the Ð_{M} for the polymer is 1.3 to 2.5, 1.3 to 2.2, 1.3 to 2.0, 1.3 to 1.8, 1.3 to 1.7, 1.3 to 1.6, 1.3 to 1.5, or 1.3 to 1.4. In one or more embodiments, the Ð_{M} for the polymer is 1.4 to 2.5, 1.4 to 2.2, 1.4 to 2.0, 1.4 to 1.8, 1.4 to 1.7, 1.4 to 1.6, or 1.4 to 1.5. In one or more embodiments, the Ð_{M} for the polymer is 1.5 to 2.5, 1.5 to 2.2, 1.5 to 2.0, 1.5 to 1.8, 1.5 to 1.7, or 1.5 to 1.6. In one or more embodiments, the M for the polymer is 1.6 to 2.5, 1.6 to 2.2, 1.6 to 2.0, 1.6 to 1.8, or 1.6 to 1.7. In one or more embodiments, the Ð_{M} for the polymer is 1.7 to 2.5, 1.7 to 2.2, 1.7 to 2.0, or 1.7 to 1.8. In one or more embodiments, the Ð_{M} for the polymer is 1.8 to 2.5, 1.8 to 2.2, or 1.8 to 2.0. In one or more embodiments, the Ð_{M} for the polymer is 2.0 to 2.5, 2.0 to 2.2, or 2.2 to 2.5.

A complex of the present disclosure includes a polymer (e.g., a homopolymer or diblock polymer) associated with an oligonucleotide cargo. The ratio of moles of the amine or ammonium containing cationic group or cationizable of the polymer to the moles of the negatively charged phosphorous containing groups (e.g., phosphate groups or phosphorothioate groups) of the oligonucleotide cargo (N/P) may vary. The N/P ratio of the polymer may affect the efficiency of the complex as a delivery system. As used herein, the N/P ratio refers to the N/P ratio of a single complex or the average N/P ratio of a plurality of complexes. It is understood that the N/P of a complex or a plurality of complexes is the predicted or calculated N/P of such complex or complexes. In one or more embodiments, the N/P ratio is 1 or greater, 3 or greater, 5 or greater, 6 or greater, 8 or greater, 10 or greater, 12 or greater, 14 or greater, 16 or greater, 18 or greater, 20 or greater, 25 or greater, or 30 or greater. In one or more embodiments, the N/P ratio is 40 or less, 35 or less, 30 or less, 25 or less, 20 or less, 18 or less, 16 or less, 14 or less, 12 or less, 10 or less, 8 or less, 6 or less, 5 or less, or 3 or less. In one or more embodiments, the N/P ratio is 1 to 40, 1 to 35, 1 to 30, 1 to 25, 1 to 20, 1 to 18, 1 to 16, 1 to 14, 1 to 12, 1 to 12, 1 to 10, 1 to 8, 1 to 6, 1 to 5, or 1 to 3. In one or more embodiments the N/P ratio is 3 to 40, 3 to 35, 3 to 30, 3 to 25, 3 to 20, 3 to 18, 3 to 16, 3 to 14, 3 to 12, 3 to 12, 3 to 10, 3 to 8, 3 to 6, or 3 to 5. In one or more embodiments, the N/P ratio is 5 to 40, 5 to 35, 5 to 30, 5 to 25, 5 to 20, 5 to 18, 5 to 16, 5 to 14, 5 to 12, 5 to 12, 5 to 10, 5 to 8, or 5 to 6. In one or more embodiments, the N/P ratio is 6 to 40, 6 to 36, 6 to 30, 6 to 26, 6 to 20, 6 to 18, 6 to 16, 6 to 14, 6 to 12, 6 to 12, 6 to 10, or 6 to 8. In one or more embodiments, the N/P ratio is 8 to 40, 8 to 36, 8 to 30, 8 to 26, 8 to 20, 8 to 18, 8 to 16, 8 to 14, 8 to 12, 8 to 12, or 8 to 10. In one or more embodiments, the N/P ratio is 10 to 40, 10 to 36, 10 to 30, 10 to 26, 10 to 20, 10 to 18, 10 to 16, 10 to 14, or 10 to 12. In one or more embodiments, the N/P ratio is 12 to 40, 12 to 36, 12 to 30, 12 to 26, 12 to 20, 12 to 18, 12 to 16, or 12 to 14. In one or more embodiments, the N/P ratio is 14 to 40, 14 to 36, 14 to 30, 14 to 26, 14 to 20, 14 to 18, or 14 to 16. In one or more embodiments, the N/P ratio is 16 to 40, 16 to 36, 16 to 30, 16 to 26, 16 to 20, or 16 to 18. In one or more embodiments, the N/P ratio is 18 to 40, 18 to 36, 18 to 30, 18 to 26, or 18 to 20. In one or more embodiments, the N/P ratio is 20 to 40, 20 to 36, 20 to 30, or 20 to 26. In one or more embodiments, the N/P ratio is 25 to 40, 25 to 36, 25 to 30, 30 to 36, or 35 to 40.

A complex of the present disclosure generally includes a plurality of polymers associated with a cargo in a particular molecular configuration. The type of molecular configuration may be dependent at least in part on the type and/or identity of polymers included in the complex. For example, a complex that includes homopolymers may be a relatively unorganized complex of a plurality of homopolymer chains and a plurality of polynucleotide chains (oligonucleotide cargo), termed a polyplex. In a polyplex, the oligonucleotide associates with (e.g., binds to) the polymer via electrostatic interactions between the negatively charged oligonucleotide backbone and the cationic groups.

In one or more embodiments, a complex that includes a diblock polymer may include or be a micelleplex. A micelleplex includes a micelle and an oligonucleotide cargo non-covalently bound to the micelle. The cargo may be non-covalently bound to the exterior of the micelle. A micelle is supramolecular assembly of a plurality of amphipathic molecules organized such that the hydrophobic (or lipophilic) portion of the molecules are packed together forming a hydrophobic core and the hydrophilic portion of the molecules are displayed exterior to the hydrophobic core. For example, the hydrophobic block of a plurality of diblock polymers of the present disclosure may assemble together to form a hydrophobic core of a micelle while the block that includes the cationic groups or cationizable groups is displayed on the surface of the micelle. The cationic groups displayed on the micelle are able to associate with (e.g., bind to) the oligonucleotide cargo via electrostatic interactions between the negatively charged oligonucleotide backbone and the cationic groups to form a micelleplex.

The size of a complex (e.g., a micelleplex or polyplex) may affect its efficacy and/or efficiency as an oligonucleotide delivery system. Complex size can be measured, for example, using dynamic light scattering such as described in Example 1 and Example 2. In one or more embodiments, a complex has a size of, or a plurality of complexes have an average size of 0.05 nm or greater, 0.5 nm or greater, 1 nm or greater, 10 nm or greater, 20 nm or greater, 30 nm or greater, 40 nm or greater, 50 nm or greater, 75 nm or greater, 100 nm or greater, or 200 nm or greater. In one or more embodiments a complex has a size of, or a plurality of complexes have an average size of 500 nm or less, 200 nm or less, 100 nm or less, 75 nm or less, 50 nm or less, 40 nm or less, 30 nm or less, 20 nm or less, 10 nm or less, 1 nm or less, or 0.5 nm or less.

The molecular configuration of the complex may affect its efficacy and/or efficiency as an oligonucleotide delivery system. Additionally, the configuration of the cells to which the complex is delivered may affect the complexes efficacy and/or efficiency as a delivery system. For example, in one or more embodiments, polyplexes may be able to more effectively delivery an oligonucleotide cargo to cells that are adhered to a surface as compared to micelleplexes. In one or more embodiments, micelleplexes may be able to more effectively delivery an oligonucleotide cargo to cells that free in solution (not adhered to a surface) as compared to polyplexes.

Nucleic acid delivery (e.g., oligonucleotide cargo delivery) is often assessed via in vitro cultures with cell monolayers. One method to enhance cellular interactions with delivery systems is to mediate aggregation, where complexes settling down on cells faster may lead to higher interactions with those cells. For example, this trend was observed when CRISPR/Cas9 ribonucleoprotein delivery using polymers in different media (without serum) was previously explored. Specifically, when CRISPR/Cas9 ribonucleoprotein polymer-complexes were formulated in PBS compared to water, some formulations had faster aggregation and settling kinetics onto the cells, which resulted in more efficient gene editing. However, there may be a limit to this phenomenon because large sizes of aggregates may affect internalization pathways and settling onto adhered cells is not a factor in the case of in vivo studies. To increase the cell interaction with complexes independent of the settling kinetics, cells can also be transfected while lifted or in suspension (for cell types that are cultured via suspension or adherent methods). Adhered cell lines can easily be exposed to transfection formulations after detachment and prior to plating. While suspension cells may be more challenging to transfect, the solution mixing can cause increased delivery system-cell interactions. Suspension cell growth methods are often used in bioreactors (for production of biopharmaceuticals) due to scale-up practicality and adherent cells can be cultured on microbeads that promote scaled culture growth in bioreactors.

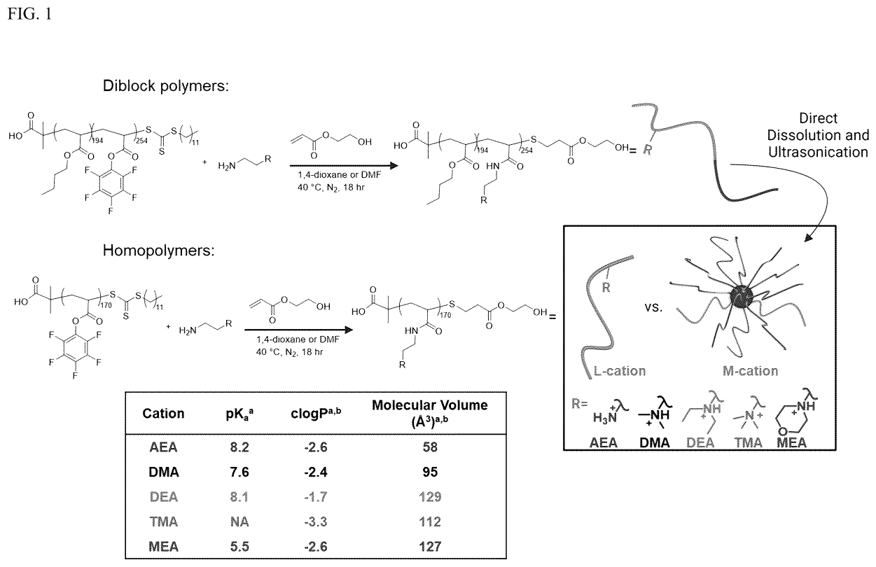

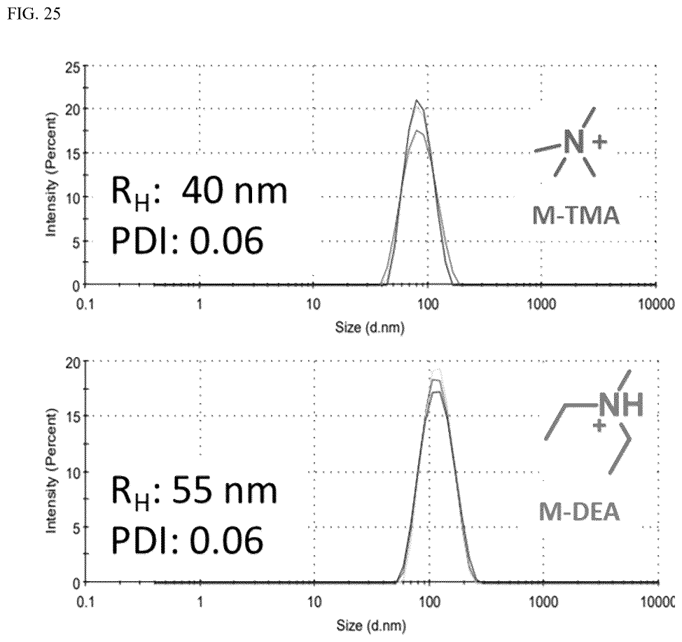

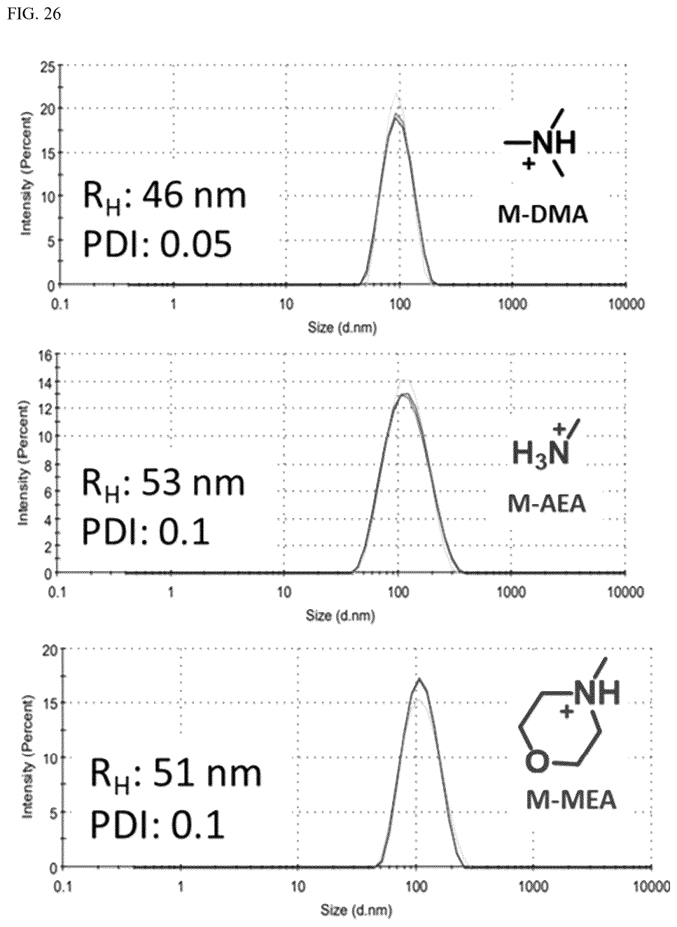

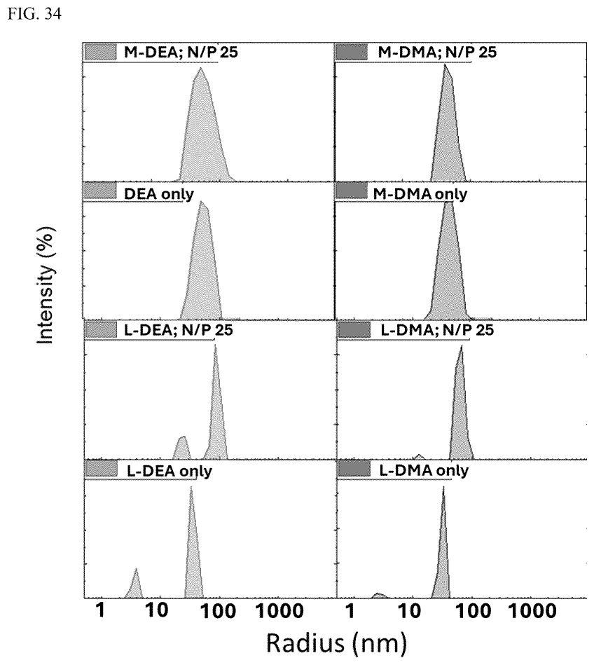

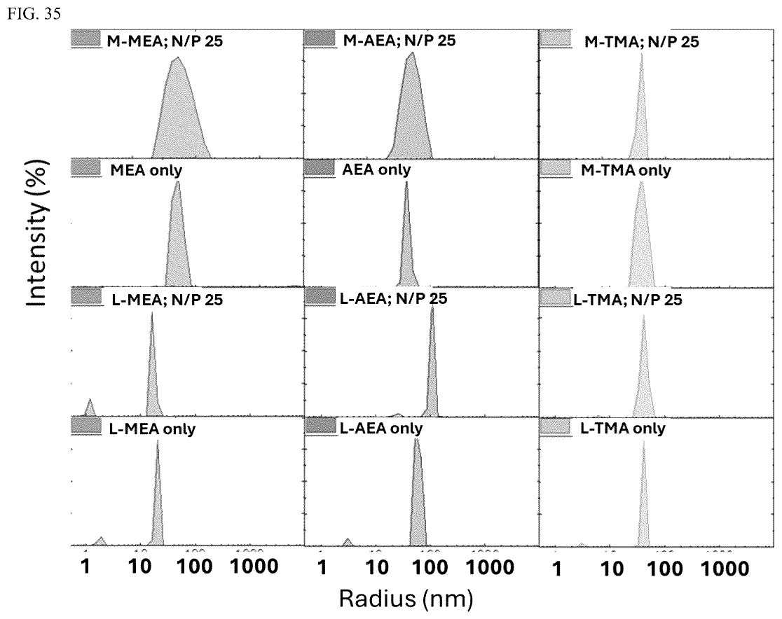

A series of 10 polymers were synthesized (see Example 1, Polymer Synthesis). A homopolymer of pentafluoro phenyl acrylate (PFPA) as well as a diblock of PFPA and a hydrophobic block of n-butyl acrylate (nBA) were synthesized to yield similar molecular weight PFPA regions. Aminolysis reactions were performed on these two scaffolds to replace the PFPA moiety with one of five different cationic groups or cationizable groups (AEA, DMA, DEA, TMA, MEA) yielding a total of 10 polymers: 5 diblock and 5 linear variants, respectively (FIG. 1). The diblock polymers were formed into unimodal micelles of ˜50 nm (see Example 1, Micelle Formation; FIG. 25, FIG. 26, FIG. 34, FIG. 35). Leveraging the modularity of the PFPA block incorporated into both the homopolymer and diblock polymer, post polymerization modifications allowed the direct comparison of the effects of each cationic group or cationizable group and their properties on ASO delivery (FIG. 1). Additionally, the effects of polymer architecture on oligonucleotide delivery was explored. Specifically, micelleplexes and polyplexes that included the same cationic group or cationizable group were compared. L-cationic abbreviation or M-cationic abbreviation will be used to state whether the architecture is linear (e.g., complexes were polyplexes) or micelle (e.g., complexes were micelleplexes), respectively along with the cationic moiety or cationizable moiety used in the polymer.

Each of the 10 linear polymers and micelles were complexed with a model ASO that knocks down deGFP production and tested in vitro to determine the delivery efficacy of each formulation. Cells were transfected either while adhered or through simultaneous addition of cells and complexes (using a lifted cell procedure) so that identical adhered cell lines could be used in both conditions (see Example 1, Transfection Study). A green fluorescent protein (GFP) knockdown assay was used to determine delivery efficacy. In the GFP knockdown assay, human embryonic kidney (HEK) cells modified to consistently express destabilized GFP (deGFP) were transfected with a polyplex or micelleplex that included an ASO that binds to the deGFP mRNA and knocks down production of the deGFP. The extent of GFP knockdown was measured using flow cytometry by quantifying the decrease in the geometric mean GFP fluorescence intensity of the cells, as compared to the untreated control. Additionally, polyplexes and micelleplexes including \a scrambled or mismatch (MM) ASO sequence were included to screen for unrelated knockdown effects deriving from the protocol and/or toxicity.

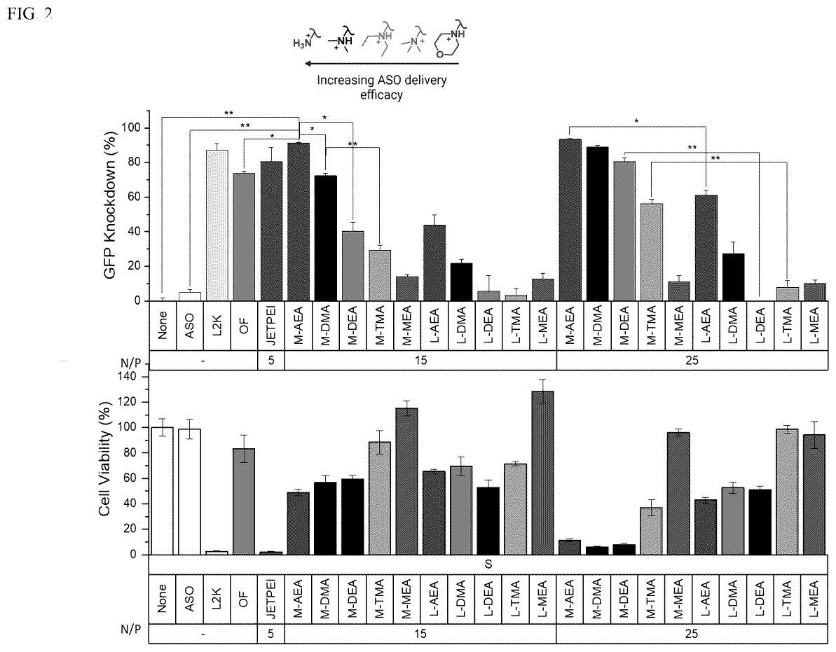

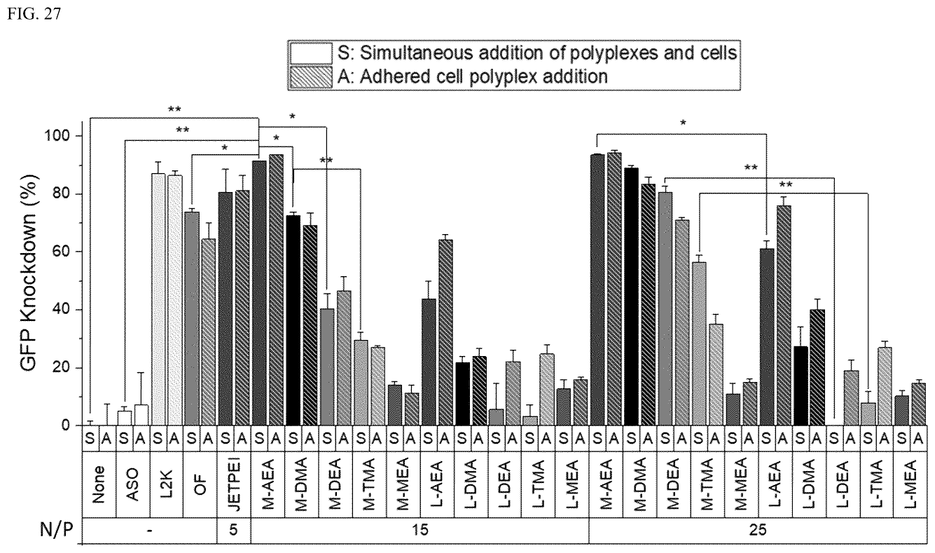

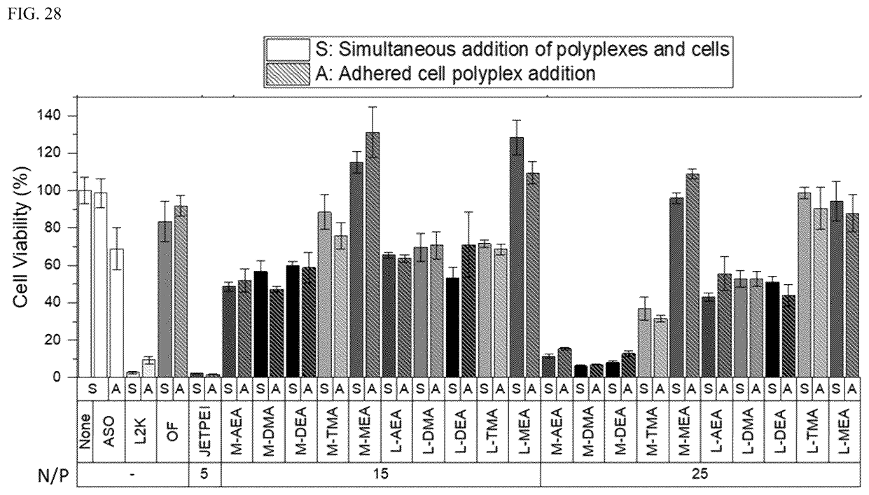

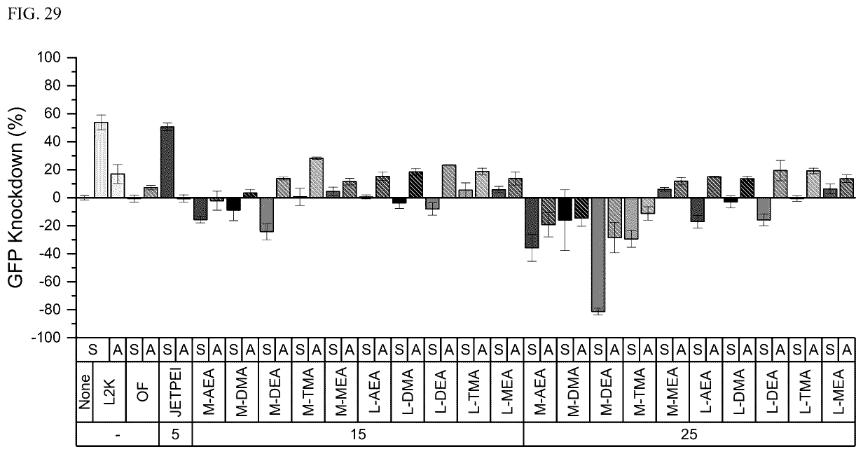

Many of the complexes exhibited good knockdown efficacy in the presence of serum (10% fetal bovine serum (FBS)) and also maintained high cell viability especially compared to the commercial controls, LIPOFECTAMINE (L2K) and Jet PEI (FIG. 2, FIG. 27, FIG. 28). OLIGOFECTAMINE (OF), another commercial control, did have significant knockdown with low toxicity, comparable to some complexes (linear polymers and micelles) of this study. The mismatch ASO (MM) negative control and had minimal knockdown (<30% except for transfection via commercial controls L2K and JetPEI) (FIG. 29). The MM control results indicated that the observed knockdown with the anti-GFP sequence ASO and the polymers of this study was due to the ASO and not toxicity or interference. However, with the commercial controls (L2K and JetPEI), toxicity is an issue as up to about 60% knockdown was found with the MM ASO. A higher toxicity indicates an increase in dead cells resulting in fewer cells that are fluorescent, which, in turn, may result in a decrease in fluorescence in the population as a whole (live+dead)(a false positive result). It is unique to have very high transfection efficacy, comparable to commercial controls for some of the micelles tested, since one downside to many polymer vehicles is their lack of stability and transfection efficacy in serum.

Micelles performed better than their linear analogues at both N/Ps studied. In each grouping of architecture and N/P there is a trend in cationic group/cationizable group performance that is similar. The primary amine, AEA, displays the highest levels of deGFP knockdown, followed by DMA, DEA, TMA, and lastly MEA. A similar trend was observed in previous studies when comparing primary, secondary, tertiary substituted amines for pDNA delivery, where less substituted amines performed better likely due to increased endosomal escape. However, these studies did not use post-polymerization methods to control for differences in length and are not comparing cations in a micelle architecture.

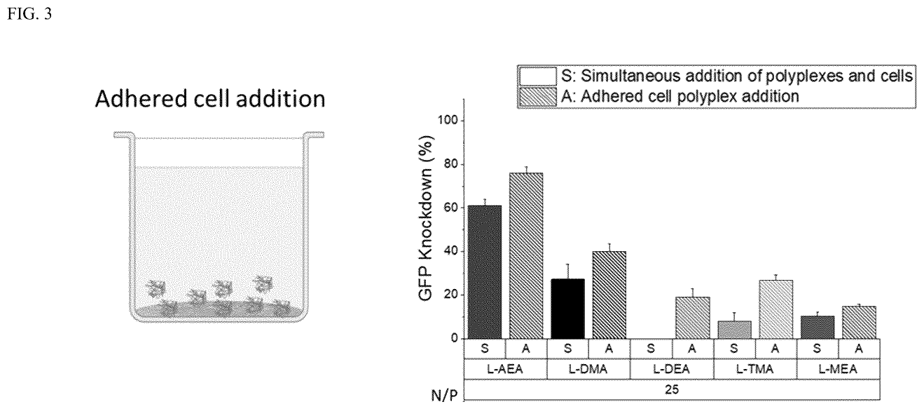

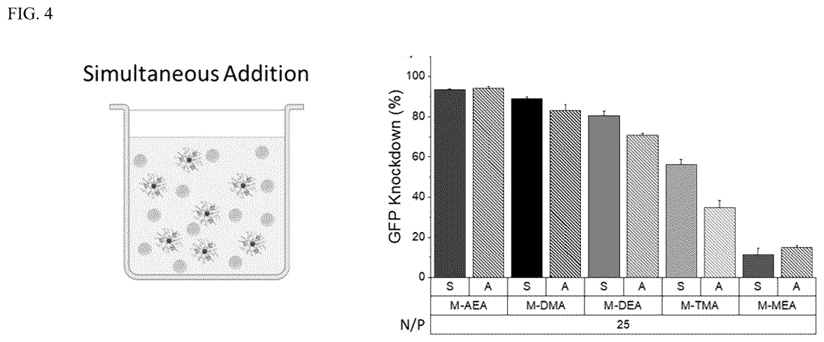

Two procedures were compared for transfection, the adhered cell and the simultaneous addition of cell and complex conditions (see Example 1, Transfection Study). Polyplex perform better when added to adhered cells compared to when cells are simultaneously added with the polyplexes. This effect may be due to linear polymers being more prone to aggregation in the presence of serum and thus have faster settling kinetics leading to an increase in cell-polyplex interaction when compared to micelleplexes (FIG. 3). Contrarily, micelleplexes perform better when cells are added simultaneously with micelleplexes compared to when added to adhered cells. This may be due to the same phenomenon as described with linear polymers; however, micelles are known to be more colloidally stable, hence their interactions with cells are maximized when cells are added at the same time and mixed as micelleplexes had slower aggregation and settling kinetics (FIG. 4).

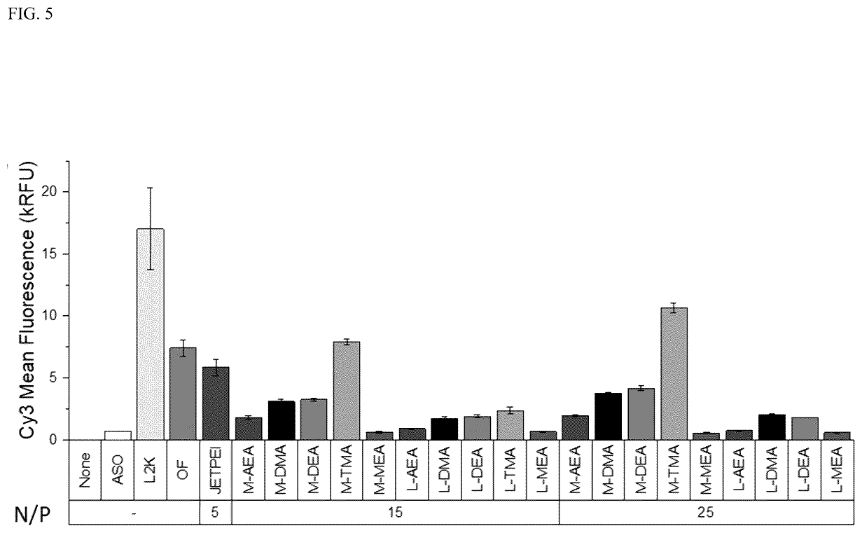

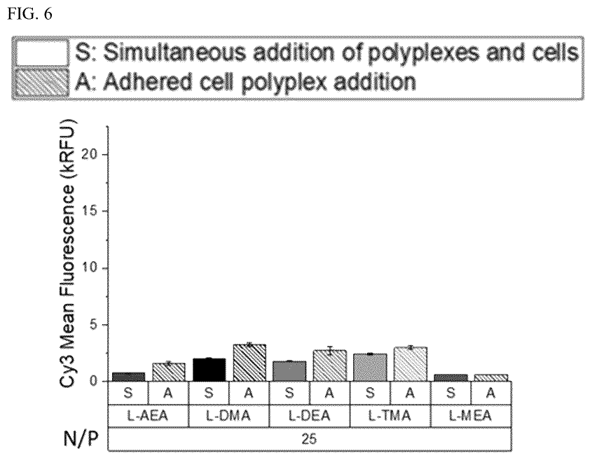

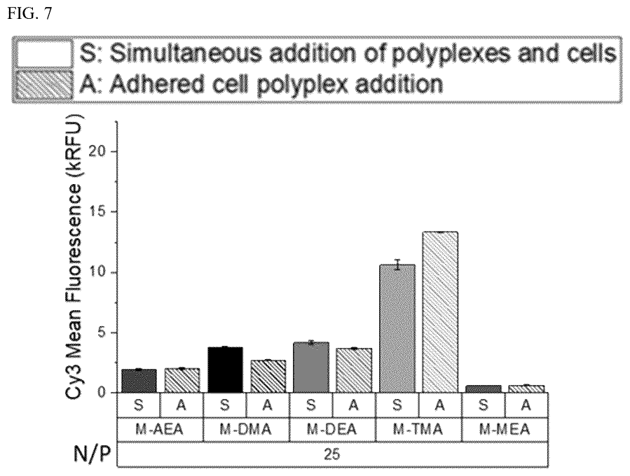

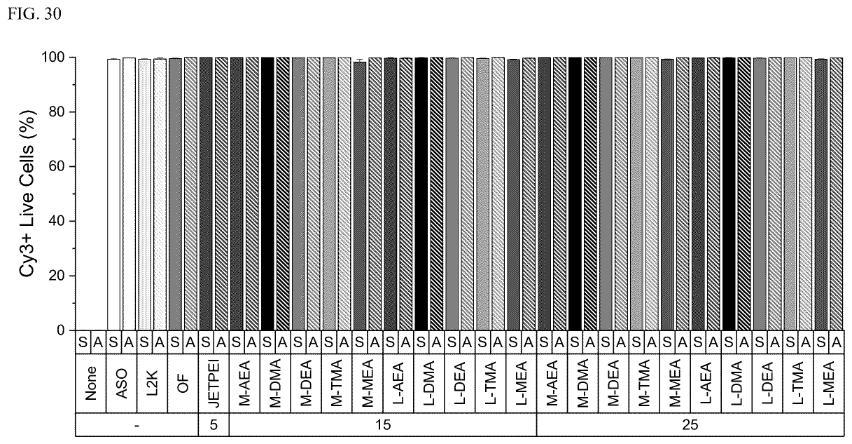

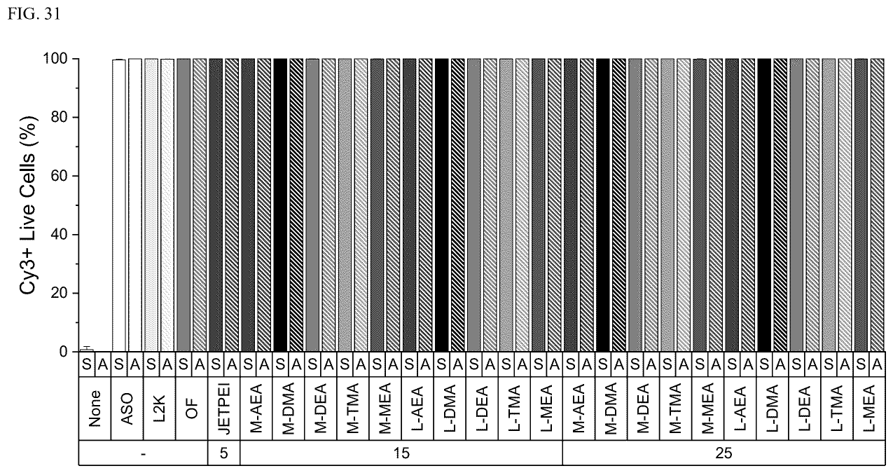

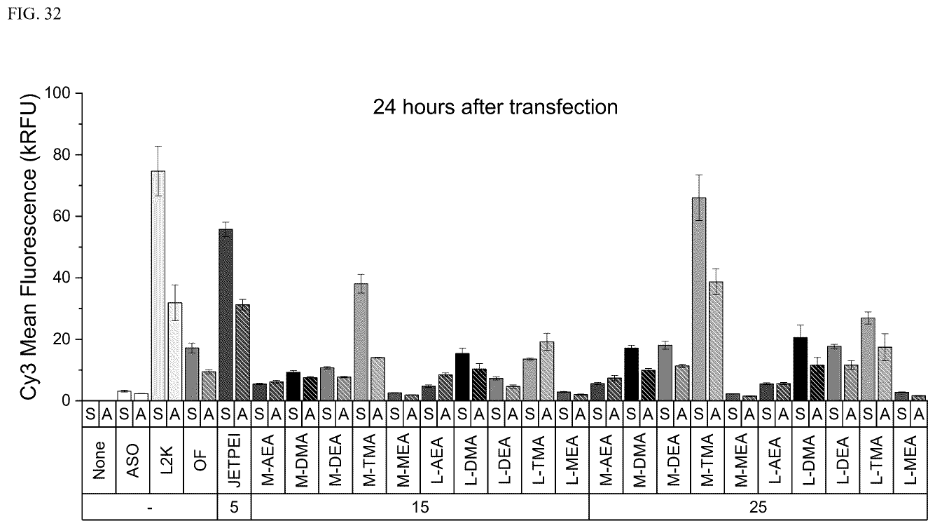

The internalization of the ASO, or lack thereof, may explain the trend seen in transfection efficiency. HEK deGFP cells were transfected in serum using a Cy3 tagged ASO (see Example 1, Cy3 ASO Internalization Study). Interestingly, the nonpackaged ASO, or ‘ASO only’, was found to be present in the full population of cells (FIG. 30, FIG. 31, and FIG. 32) likely due to the ASO modifications (phosphorothioate backbone linkages and locked rings) that help with stability and internalization. However, differences in Cy3-ASO internalization amount per cell was found to significantly vary when analyzing the Cy3 mean fluorescence intensity (FIG. 5, FIG. 6, and FIG. 7). The amount of Cy3 ASO internalization did not always correlate with deGFP knockdown. When comparing corresponding micelle and linear polymer vehicles, micelles internalize more Cy3-ASO at both the 5-hour time point in some cases (M-DEA-15, M-TMA-15&25), but not in all (M-DMA-15&25) despite having large differences in their knockdown (FIG. 5). This could be due to micelle delivery vehicles allowing better trafficking within the cell or binding of ASO to mRNA compared to linear analogues. This result is contrary to what was previously observed for pDNA delivery with micelles and linear polymers. Specifically, it was previously observed that micelles (micelleplexes included pDNA) internalized pDNA better compared to linear analogues (polyplexes including pDAN). However, the previous study only focused on one cationic/cationizable moiety for their linear polymers and micelles, DMA. Furthermore, the ASO is a different cargo than a pDNA cargo. For example, an ASO is short and single stranded as compared to pDNA that is long, double stranded, and supercoiled.

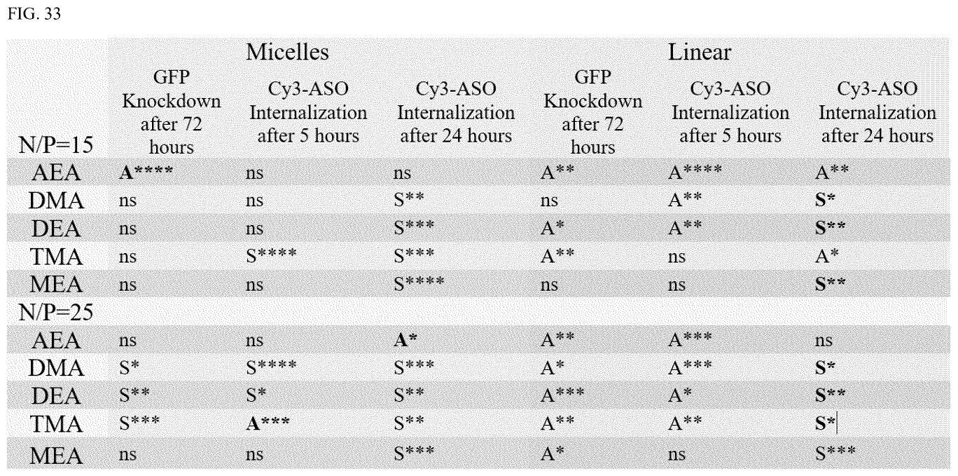

When comparing cationic/cationizable groups (excluding MEA), groups such as TMA with high internalization showed lower gene knockdown performance. This may be explained by the minimal release of ASO or minimal endosomal escape, supporting the hypothesis that internalization may not be the main factor producing high knockdown, but rather something more complex. It is important to note that Cy3 is a large hydrophobic molecule that could change the ASO properties significantly. However, it is thought that the Cy3 assay can give important insight into internalization using these delivery vehicles. When comparing adhered versus simultaneous addition probed by the Cy3 internalization assay, various trends were observed (FIG. 6, FIG. 7, FIG. 33). Generally, internalization after five hours matches the GFP knockdown after 72 hours. This might be due to higher interaction for cells that start to adhere while complexes are in solution (i.e., if cells are added simultaneously with complexes) since complexes could be caught under cells that are adhering. This would lead to cells having increased interactions with complexes that were caught under them. Since GFP knockdown is instead correlated with the five-hour time point of internalization (when comparing addition conditions), internalization may be most important over this shorter period.

Hence, micelleplexes generally have better knockdown and internalization after five hours under simultaneous addition compared to the adhered cell addition condition, and vice versa for polyplexes. This supports the hypothesis that micelles (micelleplexes) perform better under the simultaneous addition condition and linear polymers (polyplexes) perform better under adhered cell conditions due to increased cell-complex interactions and internalization.

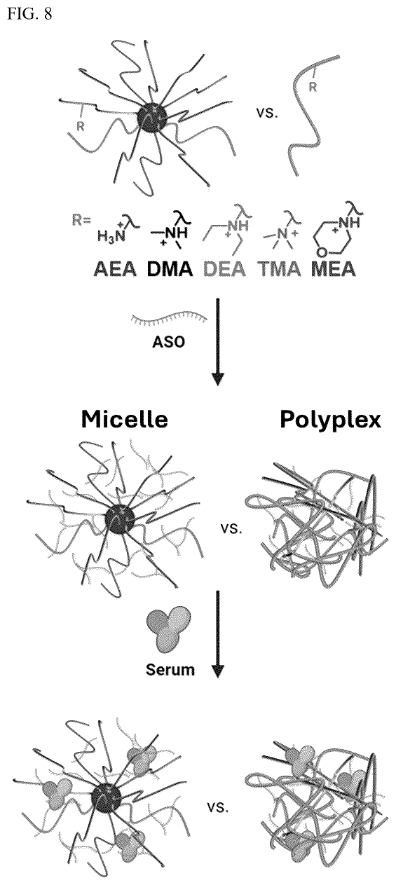

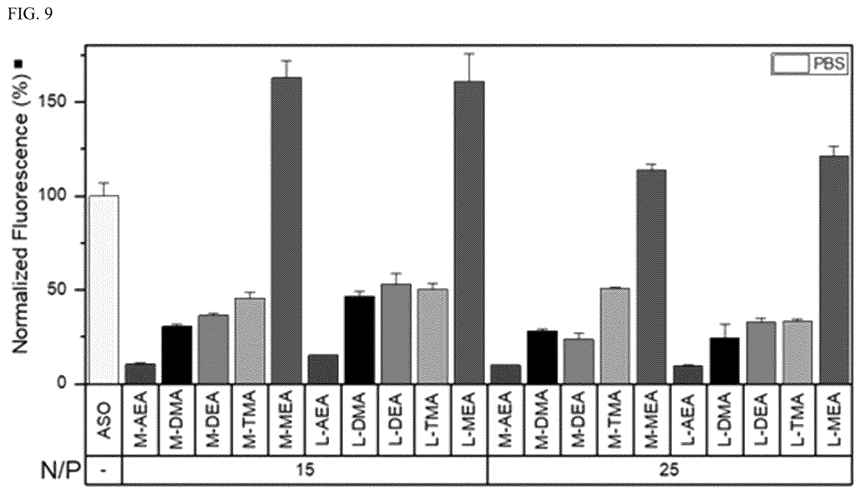

A dye exclusion binding study with QUANT-IT OLIGREEN reagent was used to compare the relative strengths of binding between the linear polymers or micelles and the ASO payload (see Example 1, Binding Study; FIG. 8 and FIG. 9). In this assay, the OLIGREEN dye binds to the ASO inducing fluorescence, however if a cationic polymer or micelle binds to the ASO and excludes the OLIGREEN dye, a decrease in fluorescence is observed. A larger decrease in fluorescence indicates more dye exclusion and tighter binding. Due to the increased number of amines (cationic groups or cationizable groups) available to bind to the ASO, generally higher N/P (25 vs 15) have higher relative binding affinity. However, the architecture does not affect the binding significantly. Interestingly, there are large differences in relative binding affinity based on the cationic/cationizable moiety. Overall, a trend was found as follows: AEA>DMA>DEA>TMA>MEA with respect to binding strength to the ASO cargo. This binding trend is also generally correlated to the number of substituents or the bulkiness of the cation (FIG. 1). Cationic groups and cations of cationizable groups, with more substituents and more bulkiness seem to generally have a lower binding affinity, likely due to steric repulsion preventing tighter binding. The poor binding of MEA may be due to its pKa (5.5) being below the pH of the solution (7.4) (FIG. 1) resulting in a lack of protonation and not allowing association (rather than its bulkiness). This trend was previously seen when looking at these micelles and their binding to pDNA. The overall trend of binding appears to correlate well with the transfection results; cationic groups that bind with a higher affinity deliver the ASO more efficiently, whereas cationic groups that bind weaker tend to have less knockdown.

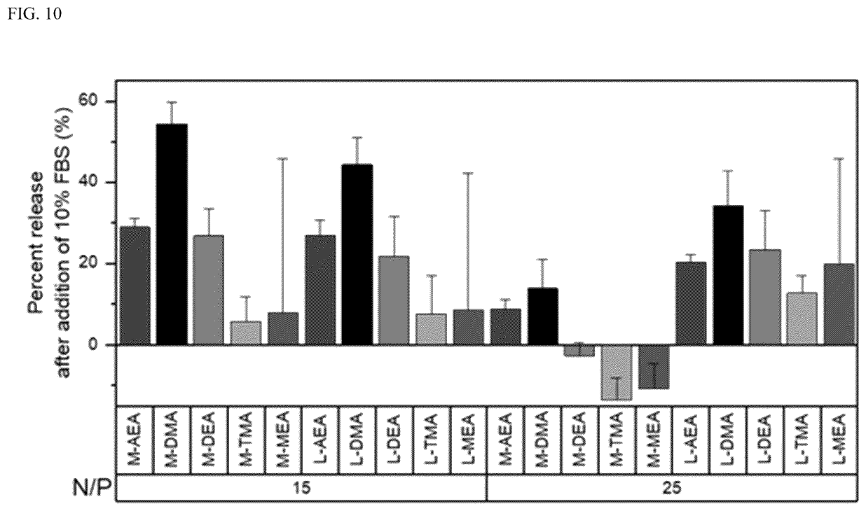

Tighter binding can be important for getting the ASO into the cell, however tighter binding can also be detrimental to delivery since the cargo must be released or unpackaged to perform its function. In this case, the ASO needs to be released from the vehicle (polymer) to bind to mRNA and cause knockdown. Adding a small amount of serum (10% FBS) to the culture media decreased the ASO-polymer binding. Interestingly, some of the cationic/cationizable groups that had stronger relative binding affinities (when no serum was present) also seemed to have increased release when exposed to FBS (such as AEA, DMA, and DEA) (FIG. 10).

Correlating these results, the delivery efficacy of polymers with different cationic/cationizable groups seems to be closely correlated to their binding strength with the ASO, thus influencing its intracellular transport. However, once in the cell, proteins at even higher concentrations (>10% serum) may help unpackage the ASO from the polymer or micelle so that the ASO can then bind to mRNA knocking down deGFP production.

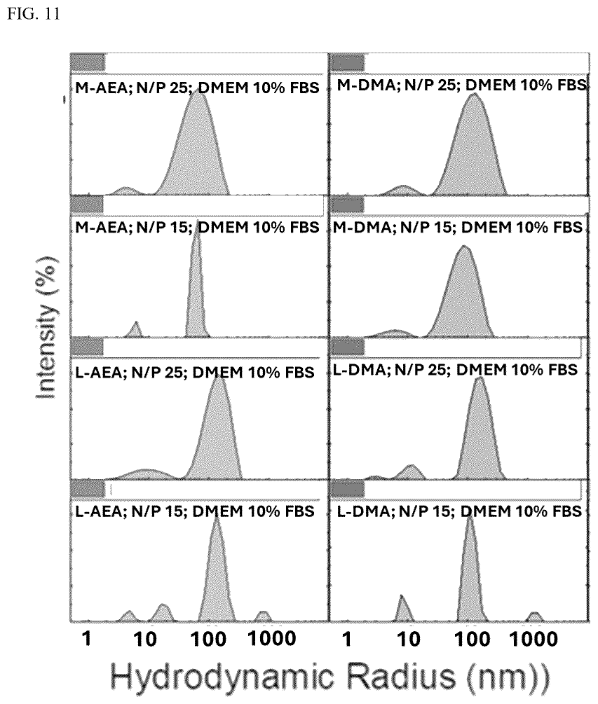

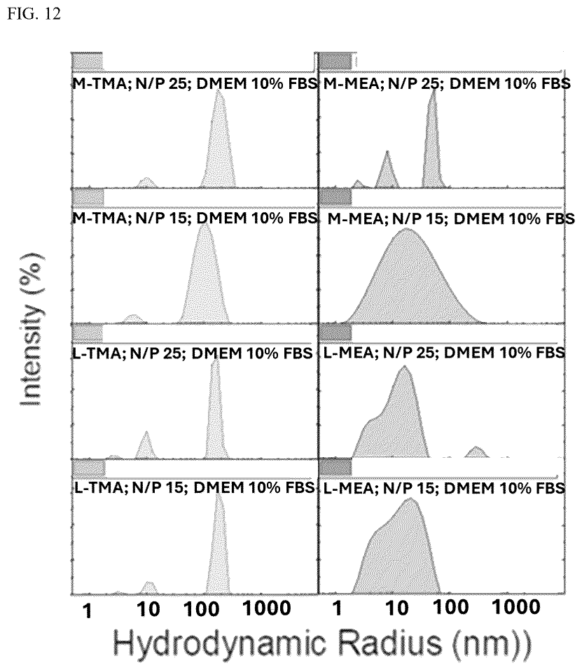

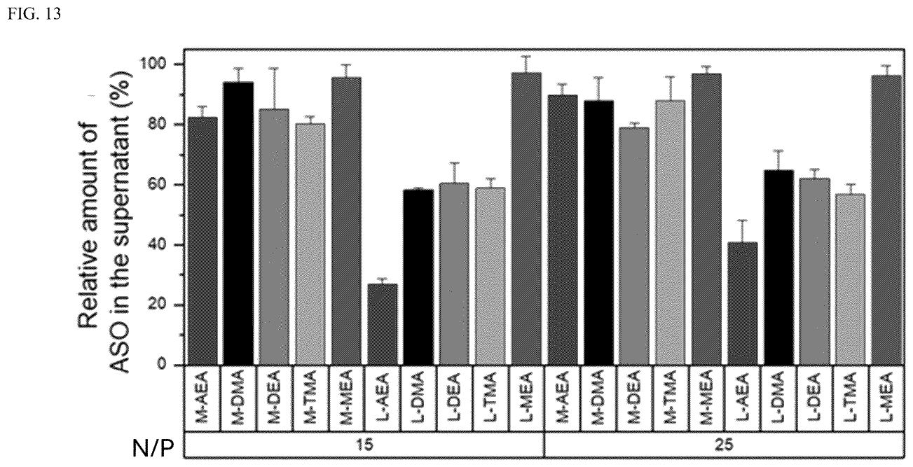

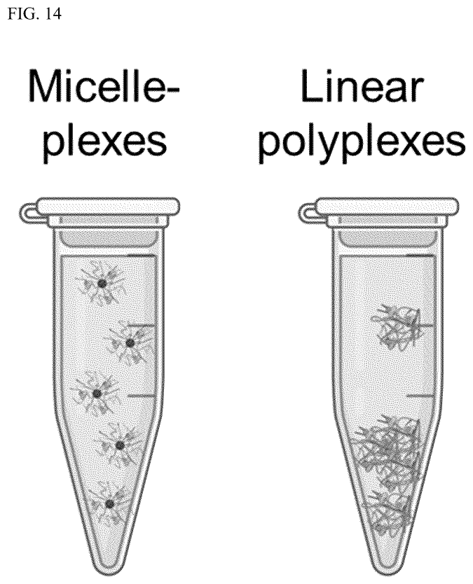

It was hypothesized that (1) polyplexes have higher aggregation causing them to settle down faster and interact with adhered cells (compared to cells that were simultaneously added) leading to increased delivery efficacy in adhered cells and (2) micelleplexes were more colloidally stable and interact with cells in the solution that were added simultaneously with micelleplexes, again leading to increased ASO delivery. To further understand the impact of solution properties, the micelleplex and linear polyplex hydrodynamic radii was measured in serum via dynamic light scattering (DLS) (see Example 1, Dynamic Light Scattering; FIG. 11 and FIG. 12). The radii of the micelleplexes and polyplexes in serum were around the same size as the micelles and linear polymers alone, as well as the micelleplexes and linear polyplexes without serum (FIG. 25, FIG. 26, FIG. 27, FIG. 28). However, the polydispersities were much higher when ASO was complexed with each respective delivery vehicle (micelle or linear polymer) relative to the uncomplexed vehicle. Additionally, the polydispersity increased even more when serum was added. This increase in polydispersity is likely due to the added components such as ASO and proteins from the serum. No significant size differences between micelleplexes and linear polyplexes in DLS studies was observed; however, it is important to note that DLS only measures the size of particles less than ˜1000 nm and only particles that remain in suspension during the analysis. To this end, it is hypothesized that not all of the complexes were measured with this experiment, particularly the population that have settled out of solution. To probe this theory, polyplexes and micelleplexes were formed with a Cy3-labeled ASO to allow a direct comparison of the differences in the concentration of ASOs suspended in the supernatant versus settled (see Example 1, Settling Study). To speed up the settling process for this experiment, centrifugation was used to pull down the population of aggregated Cy3-ASO complexes. After measuring the Cy3-ASO concentration in the supernatant, the complexes would be resuspended by vortexing the solutions to get a normalized ASO concentration ‘before settling’. Micelleplexes had a higher concentration of ASO in the supernatant, compared to their polyplex analogues (FIG. 13 and FIG. 14). This colloidal stability hypothesis is supported by previously observed transmission electron microscopy (TEM) images of similar micelleplexes where at high enough N/P ratios (>4) micelleplexes had similar sizes to the micelles without ASO likely indicating comparable colloidal stability to the micelles. Linear polyplexes were not able to be imaged via TEM due to their low contrast. Note that the MEA-based polyplexes and micelleplexes are an exception to this trend since MEA vehicles do not bind with the ASO. This study supports the hypothesis and overall data that polyplexes are less colloidally stable (particularly in serum), causing them to settle on adhered cells, leading to more interactions with cells and greater transfection efficacy. On the other hand, micelleplexes are more colloidally stable leading to better delivery in cells that have been simultaneously added with the complexes prior to plate adherence. This is an important consideration for treating difficult-to-transfect suspension cell types such as human myeloid cell line HL-60 or canine mast cell line C2 as well as for in vivo transfection when complexes are circulating in complex media such as blood, thus delineating the role of physicochemical properties in improving transfection efficiency. The colloidal stability of micelleplexes especially in serum is also useful when moving to in vivo experiments where aggregation can be detrimental to the delivery efficiency.

A complex of the present disclosure may include more than one type of polymer. A type of polymer is the composition of the polymer, for example, including the cationic or cationizable groups. In one or more embodiments, a complex of the present disclosure may include 1, 2, 3, 4, 5, 6, 7, 8, 9, or 10 different types of polymers. For example, a complex of the present disclosure may include a first polymer (e.g., homopolymer or diblock polymer) having a first cationic or cationizable group and a second polymer (e.g., homopolymer of diblock polymer) having a second cationic or cationizable group.

In one or more embodiments, a complex of the present disclosure includes a first polymer (e.g., a first diblock polymer) having a morpholino ethyl acrylamide block and a second polymer (e.g., a second diblock polymer) having an amino ethyl acrylamide, dimethyl amine ethyl acrylamide, diethyl amine ethyl acrylamide, or trimethyl amine ethyl acrylamide block.

In one or more embodiments, a complex of the present disclosure includes a first polymer having a first cation or cationizable group and a second polymer having a second cation or cationizable group. In such embodiments, the weight ratio or mole ratio of the first polymer to the second polymer can vary. It is understood that the weight ratio or mole ratio is the predicted or calculated weight ratio or mole ratio determined from the amount of the polymers present during complex or micelle formation. For example, a complex or micelle formed from a mixture that included 5 g of a first polymer and 5 g of a second polymer is said to have weight ratio of 1 part first polymer to 1 part second polymer. In one or more embodiments, the weight ratio or mole ratio of the first polymer to the second polymer is 0.1 parts or greater of the first polymer for every 1 part of the second polymer; 0.2 parts or greater of the first polymer for every 1 part of the second polymer; 0.3 parts or greater of the first polymer for every 1 part of the second polymer; 0.4 parts or greater of the first polymer for every 1 part of the second polymer; 0.5 parts or greater of the first polymer for every 1 part of the second polymer; 0.6 parts or greater of the first polymer for every 1 part of the second polymer; 0.7 parts or greater of the first polymer for every 1 part of the second polymer; 0.8 parts or greater of the first polymer for every 1 part of the second polymer; or 0.9 parts or greater of the first polymer for every 1 part of the second polymer. In one or more embodiments, the weight ratio or mole ratio of the first polymer to the second polymer is 1 part or less of the first polymer for every 1 part of the second polymer; 0.9 parts or less of the first polymer for every 1 part of the second polymer; 0.8 parts or less of the first polymer for every 1 part of the second polymer; 0.7 parts or less of the first polymer for every 1 part of the second polymer; 0.6 parts or less of the first polymer for every 1 part of the second polymer; 0.5 parts or less of the first polymer for every 1 part of the second polymer; 0.4 parts or less of the first polymer for every 1 part of the second polymer; 0.3 parts or less of the first polymer for every 1 part of the second polymer; or 0.2 parts or less of the first polymer for every 1 part of the second polymer.

In embodiments, the weight ratio or mole ratio of the first polymer to the second polymer is 0.8 parts to 1 part of the first polymer for every 1 part of the second polymer. In embodiments, the weight ratio or mole ratio of the first polymer to the second polymer is 0.1 parts to 0.5 part of the first polymer for every 1 part of the second polymer. In embodiments, the weight ratio or mole ratio of the first polymer to the second polymer is 0.1 parts to 0.3 part of the first polymer for every 1 part of the second polymer. In embodiments, the weight ratio or mole ratio of the first polymer to the second polymer is 0.2 parts of the first polymer for every 1 part of the second polymer. A complex having two or more different polymers (e.g., a first diblock polymer and a second diblock polymer) has a total polymer content. The total polymer content is the sum of the number of moles or weight of the polymers. In one or more embodiments, the total polymer content a complex having two or more different polymers includes 5 mol-% or weight-% or greater, 10 mol-% or weight-% or greater, 20 mol-% or weight-% or greater, 30 mol-% or weight-% or greater, 40 mol-% or weight-% or greater, 50 mol-% or weight-% or greater, 60 mol-% or weight-% or greater, 70 mol-% or weight-% or greater, or 80 mol-% or weight-% or greater of the first polymer. In one or more embodiments, the total polymer content a complex having two or more different polymers includes 90 mol-% or weight-% or less, 80 mol-% or weight-% or less, 70 mol-% or weight-% or less, 60 mol-% or weight-% or less, 50 mol-% or weight-% or less, 40 mol-% or weight-% or less, 30 mol-% or weight-% or less, 20 mol-% or weight-% or less, or 10 mol-% or weight-% or less of the first polymer. In one or more embodiments, a complex having two or more polymers has a total polymer content that includes 30 mol-% or weight-% to 70 mol-% of weight-%, 40 mol-% or weight-% to 60 mole-% or weight-%, or 50 mol-% or weigh-% of the first polymer. In one or more embodiments, a complex having two or more polymers has a total polymer content that includes 5 mol-% or weight-% to 40 mol-% of weight-%, 10 mol-% or weight-% to 30 mole-% or weight-%, or 20 mol-% or weigh-% of the first polymer. It is understood that the mole-% or weight-% of the polymers in the total polymer content is the predicted of calculated amount determined from the amount of the polymers present during complex formation. For example, a complex formed from a mixture that included 5 g of a first polymer and 5 g of a second polymer is said to have a total polymer content that has 50 weight-% first polymer and 50 weight-% second polymer.

In another aspect, the present disclosure describes a composition that includes one or more complexes of the present disclosure. The composition may be a delivery system composition. In one or more embodiments, the delivery system is a transfection composition. A transfection composition is a composition that includes one or more components for transfection. Transfection is the in vivo or in vitro process of introducing an exogenous oligonucleotide (e.g., an oligonucleotide cargo) into a cell.

A transfection composition includes a plurality of complexes of the present disclosure. The plurality of complexes may include one complex species or two or more complex species. It is understood that a “complex species” refers to a complex that includes a specific oligonucleotide cargo and a specific polymer or polymers (e.g., a complex that includes a first polymer and a second polymer). A first complex species may differ from a second complex species by the oligonucleotide cargo, the polymer or polymers, or both.

In one or more embodiments, a transfection composition includes a first complex and a second complex. The amount of each complex in a transfection composition may vary. In one or more embodiments, the weight ratio or mole ratio of the first complex to the second complex is 0.1 parts or greater of the first complex for every 1 part of the second complex; 0.2 parts or greater of the first complex for every 1 part of the second complex; 0.3 parts or greater of the first complex for every 1 part of the second complex; 0.4 parts or greater of the first complex for every 1 part of the second complex; 0.5 parts or greater of the first complex for every 1 part of the second complex; 0.6 parts or greater of the first complex for every 1 part of the second complex; 0.7 parts or greater of the first complex for every 1 part of the second complex; 0.8 parts or greater of the first complex for every 1 part of the second complex; or 0.9 parts or greater of the first complex for every 1 part of the second complex. In one or more embodiments, the weight ratio or mole ratio of the first complex to the second complex is 1 part or less of the first complex for every 1 part of the second complex; 0.9 parts or less of the first complex for every 1 part of the second complex; 0.8 parts or less of the first complex for every 1 part of the second complex; 0.7 parts or less of the first complex for every 1 part of the second complex; 0.6 parts or less of the first complex for every 1 part of the second complex; 0.5 parts or less of the first complex for every 1 part of the second complex; 0.4 parts or less of the first complex for every 1 part of the second complex; 0.3 parts or less of the first complex for every 1 part of the second complex; or 0.2 parts or less of the first complex for every 1 part of the second complex.

The transfection complex may further include a transfection medium. The transfection medium may be any suitable medium for contact with cells. In one or more embodiments, the medium comprises water. In one or more embodiments, the medium includes salts and or agents or compounds that promote cell health and/or growth. In one or more embodiments, the medium is a cell growth medium, such as, for example, Dulbecco's Modified Eagle Medium (DMEM), Minimum Essential Medium (MEM), RPMI 1640 Medium, OPTIMEM I, or any combination thereof. In one or more embodiments, the medium includes proteins. In other embodiments, the medium does not include proteins. In one or more embodiments, the medium is serum free. In other embodiments, the medium includes serum.

The pH of the transfection composition may vary. In one or more embodiments, the pH of the transfection composition is 2 or greater, 3 or greater, 4 or greater, 5 or greater, 6 or greater, 7 or greater, or 8 or greater. In one or more embodiments, the pH of the transfection composition is 9 or less, 8 or less, 7 or less, 6 or less, 5 or less, 4 or less, or 3 or less. In one or more embodiments, the pH of the transfection composition is 4 to 8, 5 to 8, 6 to 8, or 7 to 8. In another aspect, the present disclosure describes a method. The method includes forming a complex of the present disclosure. The method used to form the complex may depend at least in part on the polymer or polymers used to form the complex.

In one or more embodiments, the method includes mixing a polymer or polymers (e.g., one or more different types of polymers) with an oligonucleotide cargo to create a first mixture. The method may further include incubating the first mixture for a first period of time to form a second mixture comprising the complex. In one or more embodiments, the second mixture is a transfection composition that can be used to treat cells. For example, this method may be used to form a complex that includes a homopolymer. This method may be used to form a polyplex.

In one or more embodiments, the method includes mixing a polymer or polymers in a pre-first mixture. The method may further include incubating the pre-first mixture for period of time to form a first mixture that include micelles. Incubating the pre-first mixture allows at least a portion of the polymers to organize into one or more micelles. In one or more embodiments, the method includes incubating the first mixture with an oligonucleotide to from a second mixture, the second mixture including a plurality of complexes. Incubating the first mixture with the oligonucleotide allows at least a portion of the oligonucleotide to associate with the micelles to form micelleplexes. In one or more embodiments, the second mixture is a transfection composition that can be used to treat cells. For example, this method may be used to form a complex that includes a diblock polymer or two or more diblock polymers.

The pre-first mixture includes a carrier. The carrier may include water. The carrier may further include one or more additional components. For example, the carrier may include a buffering agent and/or a salt. Examples of buffering agents include 3-(N-morpholino)propanesulfonic acid; 2-(N-morpholino)ethanesulfonic acid; and 4-(2-hydroxyethyl)-1-piperazineethanesulfonic acid.

The pre-first mixture may have one or more properties that may facilitate micelle formation. For example, the pre-first mixture may have a pH of 4 to 9, such as 6.5 to 7.5 or 7. The pre-first mixture may have an ionic strength of 10 mM to 1000 mM, such as 50 mM to 200 mM or 100 mM.

In one or more embodiments, the method may further include agitating the pre-first mixture after incubation. For example, ultrasonication may be used to agitate the pre-first mixture. In one or more embodiments, the method may further include incubating the agitated pre-first mixture at an elevated temperature.

The absolute concentration of the one or more polymers and the absolute concentration of the oligonucleotide in the first mixtures of the methods presented herein may vary depending at least in part on the desired N/P ratio, the identity and properties of the polymer or polymers, the identity and properties of the oligonucleotide, the number of cells to be treated, other factors, or any combination thereof.

In one or more embodiments, the first mixture includes water. In one or more embodiments, the first mixture includes an aqueous solution of water and an acid. The acid is used to adjust the pH to a value of 1 to 5. A low pH may be beneficial to complex as a low pH may increase the probability that the cationizable groups of the polymers are ionized (e.g., protonated). Protonation of the cationizable amines gives the cationizable amines a positive charge which may increase electrostatic interactions between the polymer and any negatively charged backbone groups of an oligonucleotide. Any suitable acid or buffer may be included. Examples of acids include, but are not limited to, citric acid, acetic acid, hydrochloric acid, formic acid, lactic acid, uric acid, malic acid, tartaric acid, sulfuric acid, and the like. Examples of buffer include sodium acetate buffer, chloroacetate buffer, and the like.

The length of first incubation period of time may affect the transfection efficiency of a complex. In one or more embodiments, the first period of time is 1 minute or greater, 15 minutes or greater, 20 minutes or greater, 25 minutes or greater, 30 minutes or greater, 40 min or greater, 1 hour or greater, 2 hours or greater, 4 hours or greater, or 5 hours or greater. In one or more embodiments, the first period of time is 5 hours or less, 4 hours or less, 2 hours or less, 1 hour or less, 40 minutes or less, 35 minutes or less, 30 minutes or less, 25 minutes or less, 20 minutes or less, or 15 minutes or less. In one or more embodiments, the first period of time is 15 min to 1 hour, 15 minutes to 40 minutes, 15 minutes to 35 minutes, 15 minutes to 30 minutes, 15 minutes to 25 minutes, or 15 minutes to 20 minutes. In one or more embodiments, the first period of time is 20 minutes to 1 hour, 20 minutes to 40 minutes, 20 minutes to 35 minutes, 20 minutes to 30 minutes, or 20 minutes to 25 minutes. In one or more embodiments, the first period of time is 25 minutes to 1 hour, 25 minutes to 40 minutes, 25 minutes to 35 minutes, or 25 minutes to 30 minutes. In one or more embodiments, the first period of time is 30 minutes to 1 hour, 30 minutes to 40 minutes or 30 minutes to 35 minutes. In one or more embodiments, the first period of time is 25 minutes to 35 minutes.

In one or more embodiments, the method further includes diluting the second mixture with a dilution medium to create a third mixture. In one or more embodiments, the third mixture is a transfection composition that can be used to treat cells. In such embodiments, the dilution medium is a transfection medium.

The second mixture may be diluted with an amount of a medium such that the concentration of the complex in the third mixture is 99% or less, 90% or less, 80% or less, 70% or less, 60% or less, 50% or less, 40% or less, 30% or less, 20% or less, 10% or less, 5% or less, 1% or less 0.1% or less, 0.01% or less, 0.001% or less, 0.0001% or less of the concentration of the complex in the second mixture. In one or more embodiments, the second mixture may be diluted with an amount of a medium such that the concentration of the complex in the third mixture is 0.0001% or greater, 0.001% or greater, 0.01% or greater, 0.1% or greater, 1% or greater, 5% or greater, 10% or greater, 20% or greater, 30% or greater, 40% or greater, 50% or greater, 60% or greater, 70% or greater, 80% or greater, or 90% or greater of the concentration of the complex in the second mixture. In one or more embodiments, the second mixture may be diluted with an amount of a medium such that the concentration of the complex in the third mixture is 10% to 90%, 20% to 80%, or 40% to 60% of the concentration of the complex in the second mixture.

The length of the second period of time may affect the efficiency of transfection. In one or more embodiments, the second period of time is 1 minute or greater, 15 minutes or greater, 20 minutes or greater, 25 minutes or greater, or 30 minutes or greater, 40 minutes or greater, 1 hour or greater, 2 hours or greater, 4 hours or greater, or 5 hours or greater. In one or more embodiments, the second period of time is 5 hours or less, 4 hours or less, 2 hours or less, 1 hour or less, 40 minutes or less, 35 minutes or less, 30 minutes or less, 25 minutes or less, 20 minutes or less, or 15 minutes or less. In one or more embodiments, the second period of time is 15 minutes to 1 hour, 15 minutes to 40 minutes, 15 minutes to 35 minutes, 15 minutes to 30 minutes, 15 minutes to 25 minutes, or 15 minutes to 20 minutes. In one or more embodiments, the second period of time is 20 minutes to 1 hour, 20 minutes to 40 minutes, 20 minutes to 35 minutes, 20 minutes to 30 minutes, or 20 minutes to 25 minutes. In one or more embodiments, the second period of time is 25 minutes to 1 hour, 25 minutes to 40 minutes, 25 minutes to 35 minutes, or 25 minutes to 30 minutes. In one or more embodiments, the second period of time is 30 minutes to 1 hour, 30 minutes to 40 minutes or 30 minutes to 35 minutes. In one or more embodiments, the second period of time is 25 minutes to 35 minutes.

In another aspect, the present disclosure describes a method of transfecting a cell with a complex of the present disclosure. In some such embodiments, the complex is in a transfection composition of the present disclosure. The complex and/or transfection composition may include any components as disclosed herein and may be formed according to any method disclosed herein. The method includes contacting a complex and/or a transfection composition with a cell to form a transfection mixture. In one or more embodiments, the transfection composition is the third mixture following the second incubation time from the method of forming the complex described herein.

The technique of contacting a complex and/or transfection composition with a cell may depend at least in part on if the cell is a part of an in vitro or in vivo system. In one or more embodiments where the cell is a part of an in vitro system, contacting the cell with the complex of a transfection composition may include placing the transfection composition on top of an adhered cell or in a mixture that includes non-adhered cells. In one or more embodiments where the cell is a part of an in vivo system, contacting a complex of a transfection composition with a cell may include administering the transfection composition to a subject. Administration may be, for example, topical, enteral, or parenteral.

The pH of the complex or transfection composition used to treat the cells may affect the efficiency of transfection. For example, a pH that is too high or too low may kill the cells. In one or more embodiments, the pH of the complex or transfection composition is between 5 and 8. In one or more embodiments, the pH of complex or transfection composition is between 7 and 8.

In one or more embodiments, the method further incudes incubating the transfection mixture for a transfection time. For example, in embodiments where the cell is a part of an in vitro system, the transfection composition may be in contact with the cell for a transfection time. In one or more embodiments, the transfection time is 1 minute or greater, 15 minutes or greater, 20 minutes of greater, 25 minutes or greater, or 30 minutes or greater, 40 minutes or greater, 1 hour or greater, 5 hours or greater, 12 hours or greater, 24 hours or greater, 48 hours or greater, 64 hours or greater, or 96 hours or greater. In one or more embodiments, the transfection time of time is 96 hours or less, 64 hours or less, 48 hours or less, 24 hours or less, 12 hours or less, 5 hours or less, 1 hour or less, 40 minutes of less, 35 minutes or less, 30 minutes or less, 25 minutes or less, or 20 minutes or less. In one or more embodiments, the transfection time of time is 15 minutes to 40 minutes, 15 minutes to 35 minutes, 15 minutes to 30 minutes, 15 minutes to 25 minutes, or 15 minutes to 20 minutes. In one or more embodiments, transfection time is 20 minutes to 40 minutes, 20 minutes to 35 minutes, 20 minutes to 30 minutes, or 20 minutes to 25 minutes. In one or more embodiments, the transfection time is 25 minutes to 40 minutes, 25 minutes to 35 minutes, or 25 minutes to 30 minutes. In one or more embodiments, the transfection time is 30 minutes to 40 minutes or 30 minutes to 35 minutes. In one or more embodiments, the transfection time is 25 minutes to 35 minutes.

In one or more embodiments, the method further includes quenching the transfection mixture. For example, in one or more embodiments where the cell is a part of an in vitro system, the method further includes quenching the transfection mixture. Quenching the transfection mixture decreases the likelihood of transfection. Quenching the transfection mixture may increases cell survival during and/or after the transfection process. In one or more embodiments, quenching the transfection mixture includes adding serum-containing media to the transfection mixture to create a quenched mixture. The serum in the serum-containing media disrupts the ability of the transfection complex to be transfected into a cell.

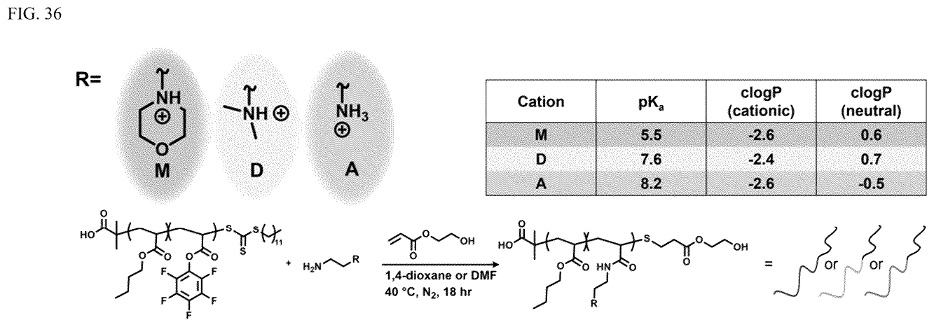

Mixed and blended micelleplex formulations were explored. Mixed micelleplexes were compositions that included two micelleplex species differing by the identity of the diblock polymer. A blended micelleplex was a single micelleplex that included two different diblock polymers, each diblock polymer having a different cationic/cationizable group. It was hypothesized the mixed and blended formulations of polymers could influence and improve properties involved in nucleic acid delivery and offer a new facile method to study and rapidly optimize polymeric formulations in a manner similar to lipids. To this end, three diblock polymers were synthesized via post polymerization modifications of a pentafluoro phenyl acrylate and n-butyl acrylate (PFPA-nBA) diblock polymer to include 3 different amine types (M, D, and A in FIG. 36; see Example 2, Diblock Synthesis) while maintaining consistent polymer length, dispersity, and block ratio between all of the three variants.

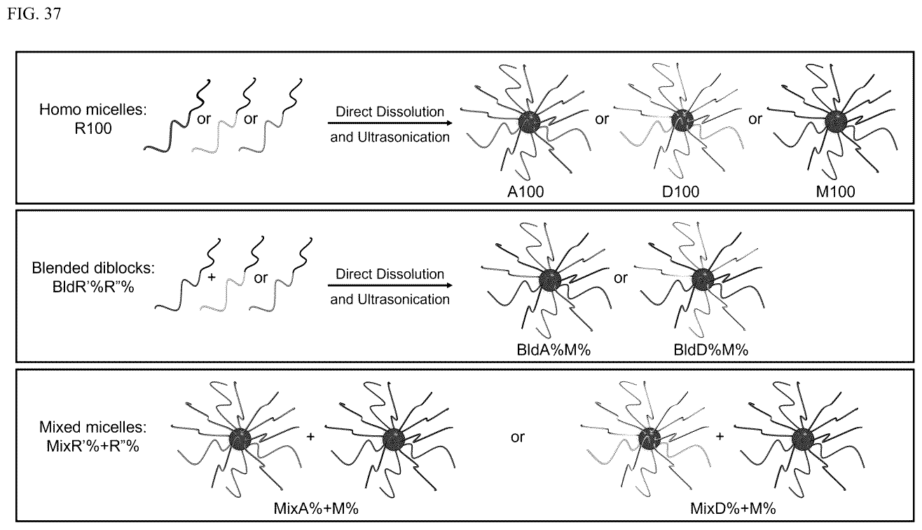

Once synthesized, the diblocks were formulated into micelles using direct dissolution in 3-(N-morpholino)propanesulfonic acid (MOPS) buffer (pH=7). They were stirred for 1 week, followed by ultrasonication for 2 hours (see Example 2, Homomicelle Formation and Blended Diblock Formation). Two different versions of micelles were formulated (1) homo-micelles, formed from one type of diblock (M100, D100, and A100) and (2) blended diblock micelles, where 2 different diblocks were blended into one micelle (BldR′% R″% where R′% and R″% indicate the amount of the diblock containing cationizable moieties R′ or R″, where R′ and R″ are different and are independently M, D, or A) (FIG. 37). For example, A100 is a micelle composed of 100% diblock that includes cationizable moiety A, where BldA50M50 refers to a blended micelle composed of 50% block that includes cationizable moiety A and 50% diblock that includes cationizable moiety M. Homomicelles were combined into mixed micelle compositions (designated as MixR′%+R″% where R′% and R″% indicate the amount of the homomicelle having cationizable moieties R′ and the amount of the homomicelle having cationizable moiety R″, where R′ and R″ are different and are independently M, D, or A) (FIG. 37). For example, a MixA50+M50 refers to a mixed micelle composition having 50% A homomicelles and 50% M homomicelles. These micelles were complexed with a model ASO to form micelleplexes and studied for their ASO delivery efficacy in serum with a model cell line. Note that blended micelleplexes and mixed micelleplexes has multiple N/Ps: an N/P of each cationizable amine type (A, D, or M) as well as the total N/P (sum of the N/Ps of each cationizable amine type).

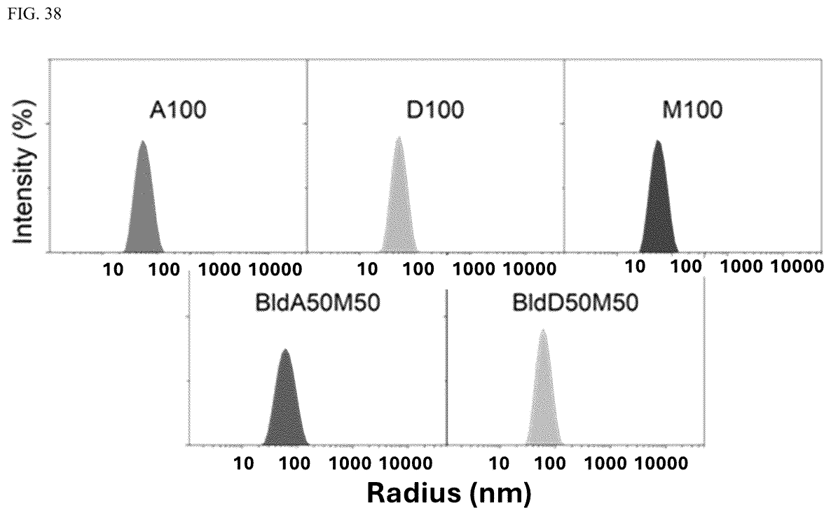

All of the micelles were of similar size of about 50-60 nm radius and low polydispersity (FIG. 38; see Example 2, Dynamic Light Scattering). This is likely due to the polymers being made from a consistent scaffold so each of the blocks are of equal size and have similar properties, indicating the micelles formed will likely be similar. Additionally, previous studies have observed that similar diblock polymers will form similarly sized micelles in aqueous solutions independent of formulation method. Thus, it was hypothesized that due to the diblocks having the same length, block ratio, and dispersity, the blended micelles would have a statistical distribution of different polymers at the stated formulation ratio. Additionally, it was thought that the mixed micelle systems would stay as two homo-micelles in the same solution within the time frame and temperature of transfection experiments (less than 1 day, 37° C.) due to the low rate of chain exchange and concentrations many magnitudes above their critical micelle compositions.

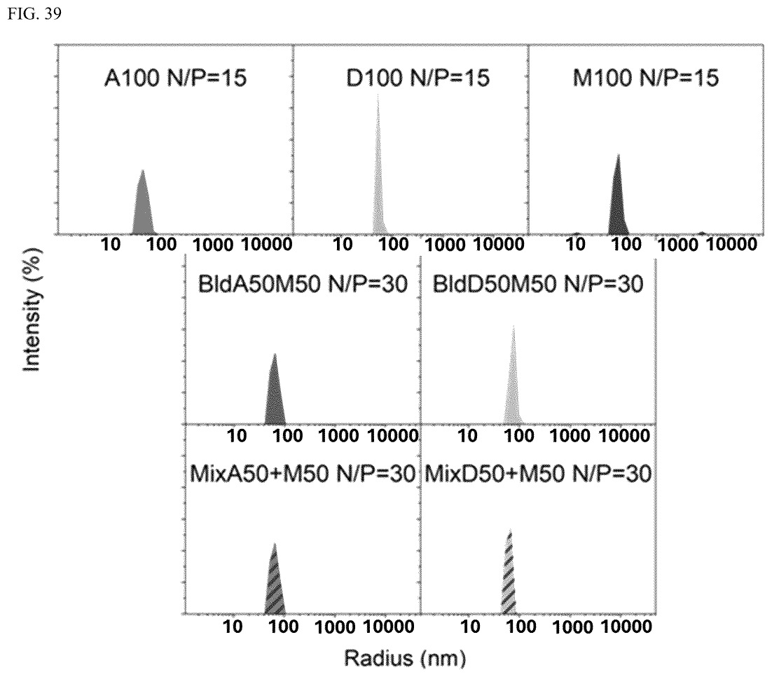

Micelleplexes were formulated at N/Ps of 15 for each homomicelle formulations (A100, D100, and M100) and N/P of 30 for blended diblock formulations (BldA50M50 and BldD50M50) and mixed micelle formulations (MixA50+M50 and MixD50+M50). These N/Ps were chosen based on the high performance of N/P 15 of micelle formulations in previous studies. Additionally, the N/P of each cationizable moiety was 15 leading to a total N/P of 30, as each cationizable moiety was in a 50:50 mixture in the mixed micelle and blended diblock system. When the micelles were complexed with ASO, it was observed that they formed unimodal micelleplex populations that mimic the size of the uncomplexed micelles (FIG. 39). It was noted that the polymers do not aggregate in the presence of ASO at the tested N/Ps. Lack of aggregation indicates that the micelles are colloidally stable, a parameter to consider for nucleic acid delivery applications in vivo. Micelleplex size may impact the oligonucleotide cargo delivery effectiveness. For example, it has been observed that micelleplex size may be a way to target certain organs.

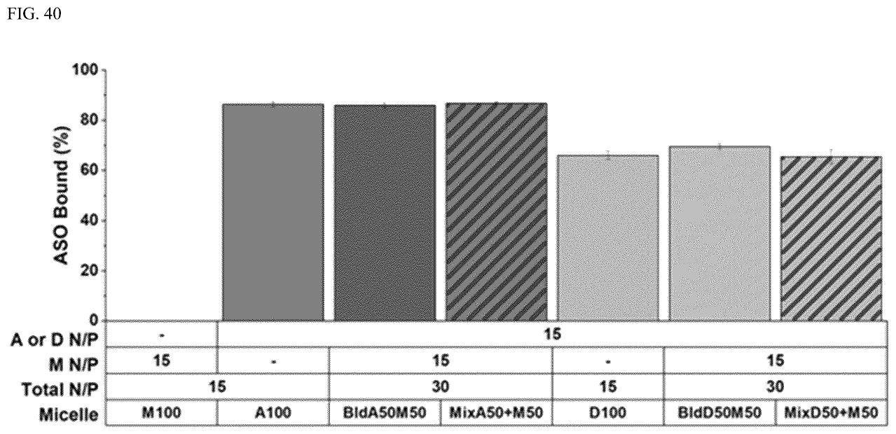

The binding of micelles to the ASO in PBS was studied (FIG. 40). To study this, a dye exclusion assay was used where an ASO-compatible dye, OLIGREEN, was added to the micelleplex solutions. If the ASO is not bound to the micelle, it is free to bind with the dye, causing the dye to fluoresce. Raw fluorescence data was normalized by dividing the micelleplex fluorescence by the ASO only control to obtain the percent ASO unbound. Next, the percent ASO unbound was subtracted from 100% to yield the percent ASO bound, where 100% bound means there was no fluorescence (no dye could bind to the ASO and fluoresce). At an N/P of 15, M100 did not bind at all to the ASO due to its lack of positive charges in PBS. A100 and D100 have strong binding to the ASO at approximately 85% and 70% respectively. When M is blended with A or D as diblocks (BldA50M50 or BldD50M50) or as mixed micelles (MixA50+M50 or MixD50+M50) the binding does not differ from the homomicelle formulations of A100 or D100, respectively. This is likely because M does not bind to the ASO, so addition of M is not affecting the binding. Polyion binding is entropically driven by the release of counterions. Because M is not charged and has no counterion on its own or in the presence of A or D, there is no driving force for the ASO to bind to M. However, it was unexpected that the presence of M in the mixed and blended formulations (BldA50M50, BldD50M50, MixA50+M50, or MixD50+M50) did not affect binding by changing the properties of the A or D moieties. Adding spacers can modify the pKa of polymers and affect binding. It is postulated that there is already plenty of excess positive charges (due to the high N/Ps), so binding appears to not be affected by changes in pKa and is affected more by the moiety substitution pattern and steric hindrance.

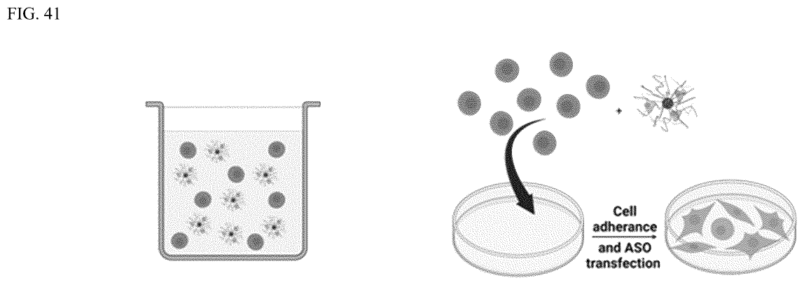

The micelleplex systems were studied for their delivery of ASOs in vitro. A GFP knockdown assay was used to study delivery efficacy (see Example 2, Transfection). Briefly, HEK cells that have been modified to continually produce destabilized GFP (deGFP) were lifted using trypsin and then added to micelleplexes containing an ASO that would knockdown deGFP mRNA and prevent it from being translated into the protein. These transfections were all completed in conditions containing serum. The cells and micelleplex formulations were added to plates allowing the cells to adhere. 72 hours after transfection, these cells were analyzed via flow cytometry to measure GFP fluorescence and corresponding gene knockdown. It has been observed that colloidally stable micelleplexes interact more when cells are temporarily suspended in solution and yield better transfection efficacy compared to cells that were pre-adhered and then transfected (FIG. 41 left). Therefore, an assay where micelleplexes were added to lifted cells rather than pre-adhered cells to increase cell-micelleplex interactions was used (FIG. 41 right).

A100 and D100 were chosen because they were the best-performing micelles in previous studies. Additionally, the moiety M was also explored as it was hypothesized that it could improve transfection. For example, it was hypothesized that the incorporation of a moiety with a lower pKa would only be protonated under acidic conditions such as endosomal pH and not in extracellular conditions therefore potentially improving endosomal escape without detrimental interactions with the cell membrane, which often causes of toxicity.

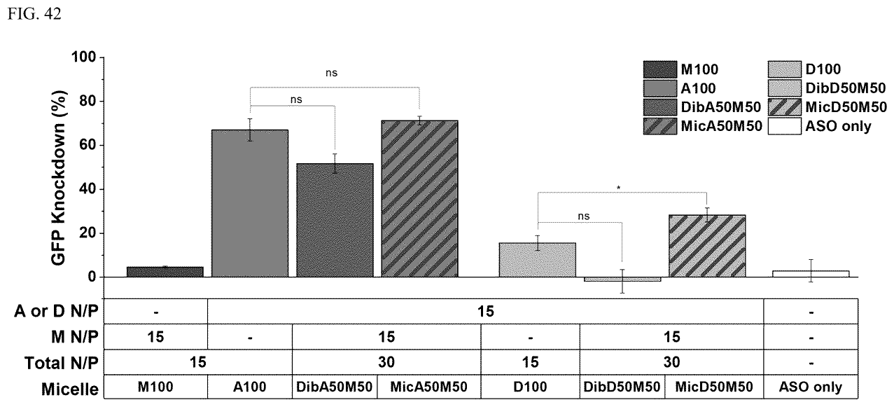

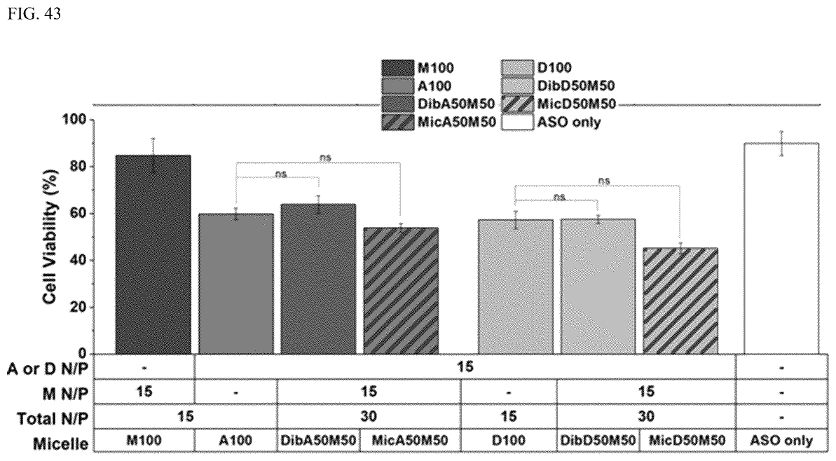

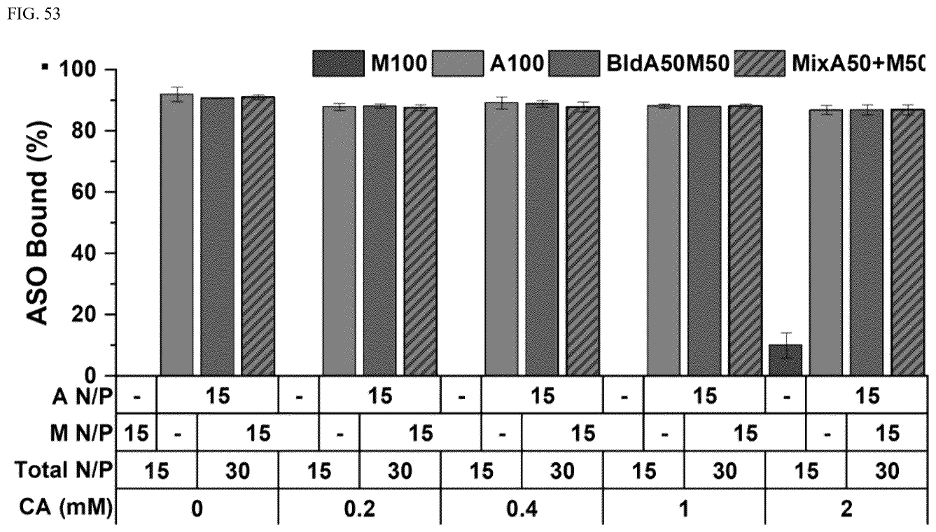

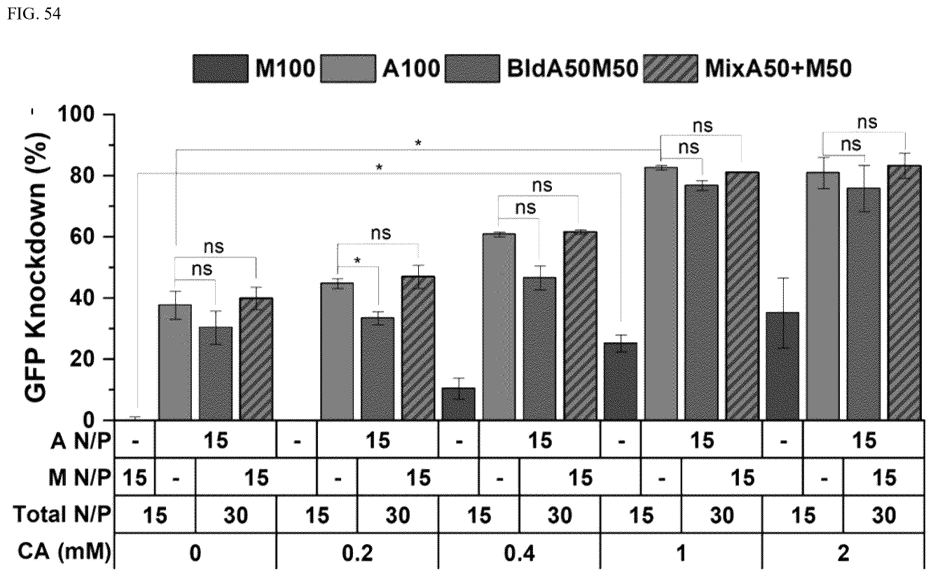

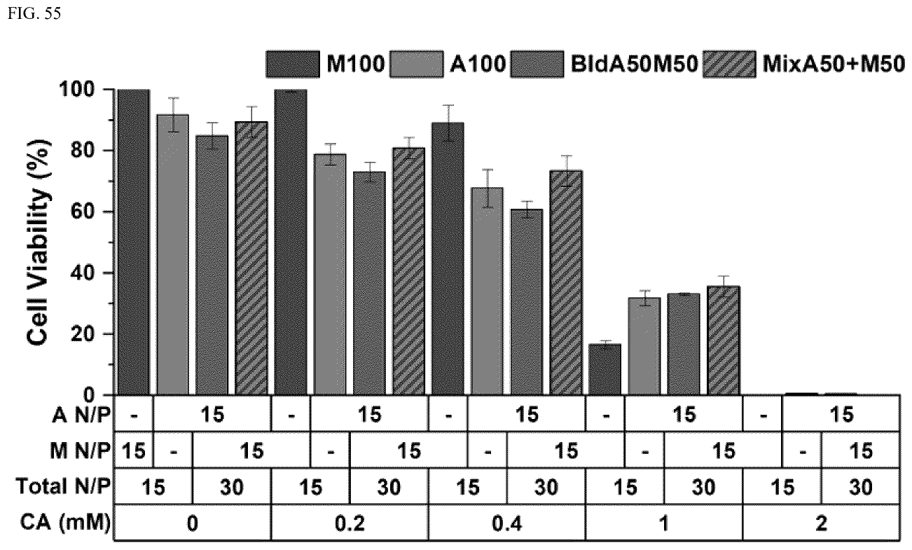

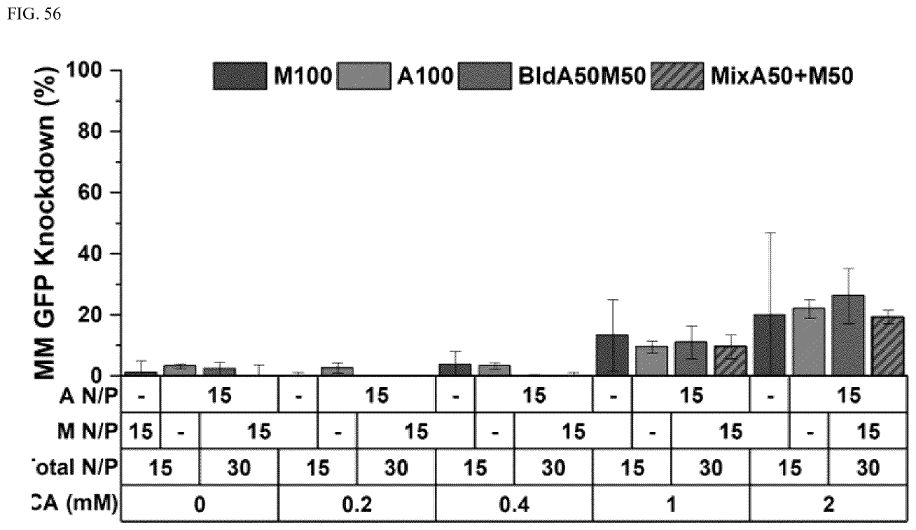

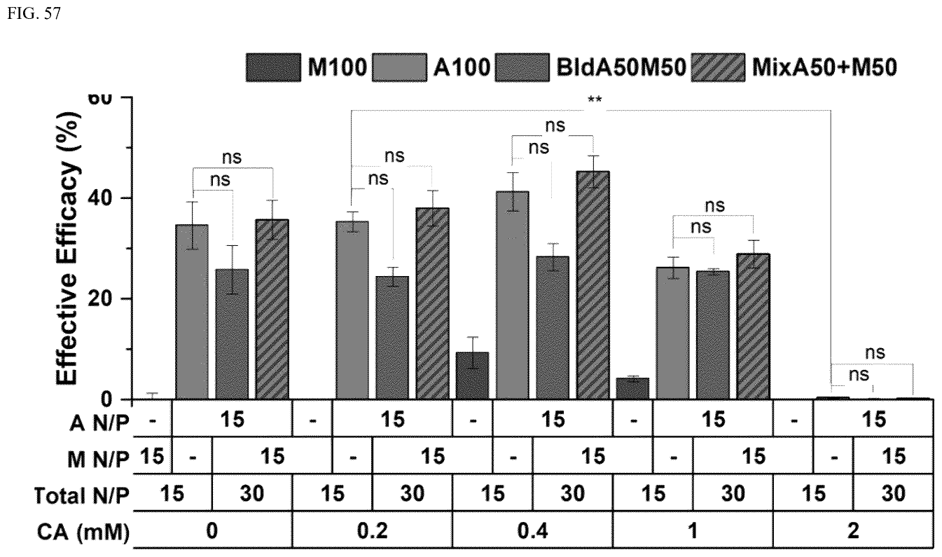

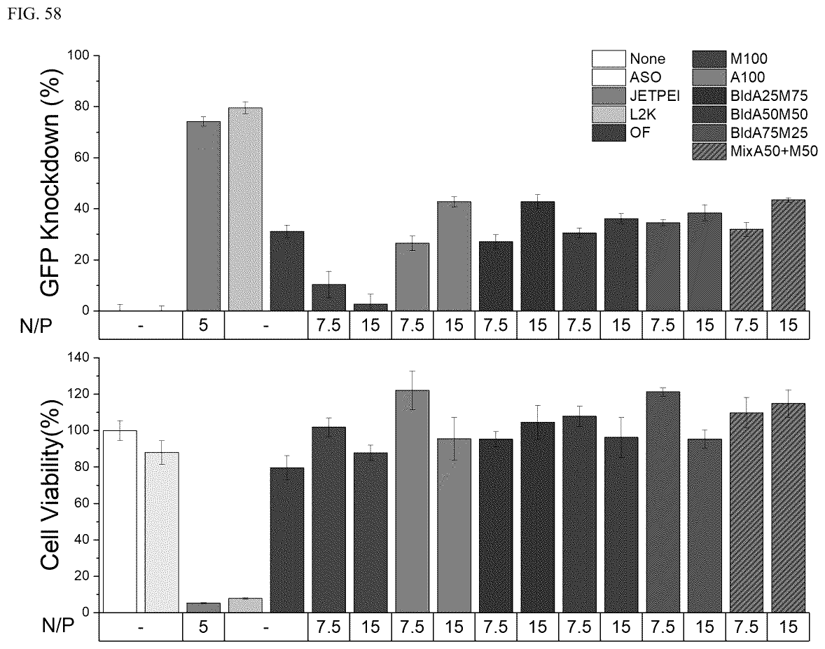

A100, D100, M100 and 50:50 formulations of M with either D or A as either blended (BldA50M50 or BldD50M50) or mixed (MixA50+M50 or MixD50+M50) micelles were first studied (FIG. 42 and FIG. 43). An N/P of 15 for A100, D100, and M100 was chosen since this N/P had high GFP knockdown and low toxicity in previous studies. For the mixed systems, the total N/P was 30 (A, D, and M each having a N/P of 15) so that it could easily compare to the A100 and D100 homomicelles. The M homomicelle complexed with ASOs had very little knockdown and low toxicity. This is hypothesized to be due to its low pKa and subsequent charge neutrality under most physiological conditions. Also, as seen before, the homomicelle A100 yielded significant transfection efficacy with decent viability. It was unexpected that there was no significant difference in GFP knockdown or viability between homomicelle A100, MixA50+M50, and BldA50M50, however MixD50+M50 had improved GFP knockdown compared to D100 with no significant decrease in viability. It was examined if M could affect A in acidic environments (chloroacetate), however there was still no improved transfection of BldA50M50 or MixA50+M50 over A100 under these conditions (FIG. 53, FIG. 54, FIG. 55, FIG. 56. and FIG. 57; see also FIG. 58). Interestingly though, chloroacetate did improve transfections for all formulations equally. However, when looking at the effective efficiency (multiple of knockdown and cell viability), chloroacetate did not improve overall performance for all systems as the increase in knockdown was followed by a related decrease in cell viability.

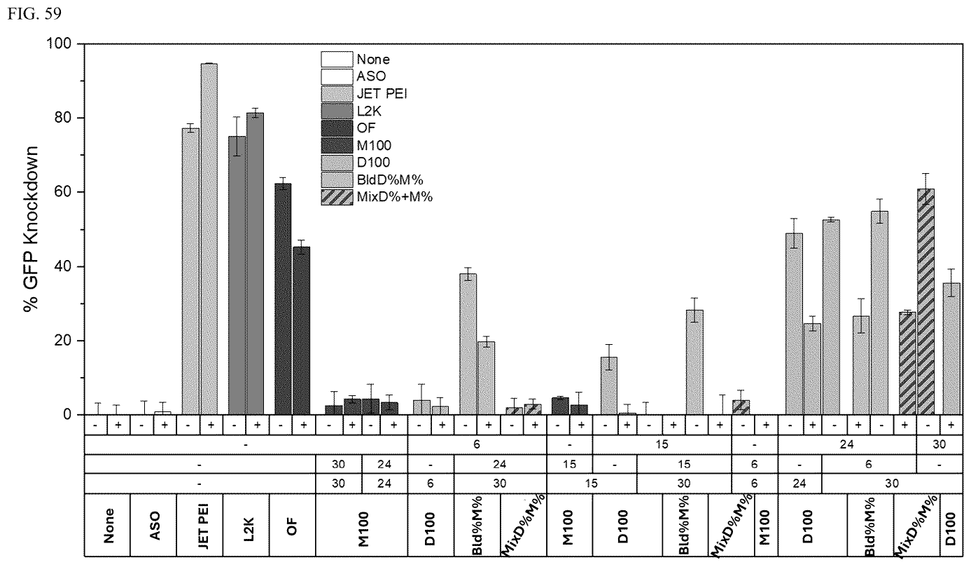

Coformulating M with D increased transfection, but both mixing or blending of M with A did not increase transfection compared to transfections of D100 and A100, respectively (FIG. 59). This may be due to the pKa of D being closer to the pH of biological conditions (pH˜7) (FIG. 36) that could have an effect on endosomal escape (acts as a proton sponge). Furthermore, it was thought that the morpholino group in M could be acting as a hydrophobic moiety (when unprotonated) that lowers the pKa of D. Since D's pKa is much closer to the pH of the conditions used, slight changes in pKa would greatly affect the percentage of D that is protonated. This could potentially lead to the increase in transfection efficacy see in MixD50⁺M50 compared to D100. The pKa of A, on the other hand, is 8.2, therefore small changes in this pKa would not significantly change the amount of A that is protonated and cause changes in the transfection.

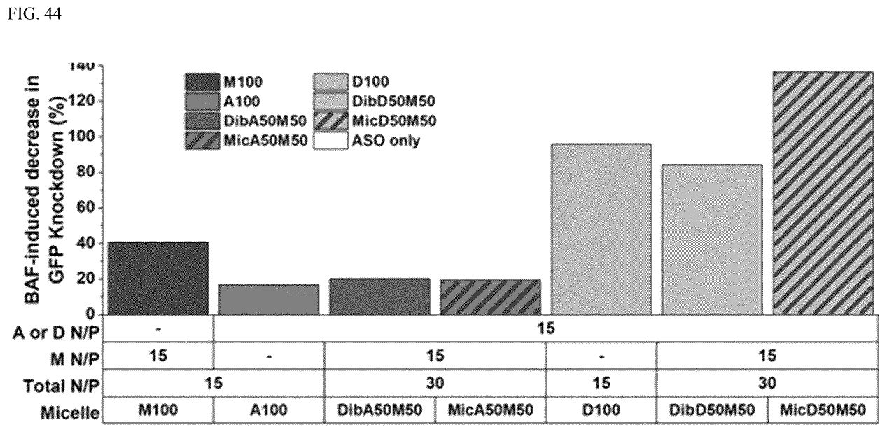

The shifts in pKa may alter the micelles' ability to escape the endosome via the proton sponge effect. To test the hypothesis that mixing or blending M with D causes more endosomal escape then mixing or blending M with A, the same transfection conditions with an additional preincubation step with Baf-A1, a small molecule known to inhibit the endosomal proton pumps that are necessary for the proton sponge effect (FIG. 44), was done. In general, micelles containing D (D100, BldD50M50, and MixD50⁺M50) rather than A (A100, BldA50M50, and MixA50⁺M50) had a larger decrease in knockdown when Baf-A1 was added (compared to standard transfection conditions in the absence of Baf-A1). This indicates that micelles containing D likely rely more on the proton sponge effect to escape the endosome as ASO-mediated knockdown is not as effective when Baf-A1 is present (because the micelles are not able to use the proton sponge effect to escape the endosome). Interestingly, it seems that the best performing micelle with D in this series, MixD50+M50, was inhibited the most with Baf-A1 addition, indicating that it heavily relies on a proton sponge-based escape. Collectively, these results support the hypothesis that M likely modulates pKa. For micelles containing D this modulation can increase transfection (for example MixD50+M50) through an increased proton sponge-based endosomal escape. However, it seems that for harder charges such as A, which has a higher pKa, no further modulation in endosomal escape and hence transfection is found with an increase in M.

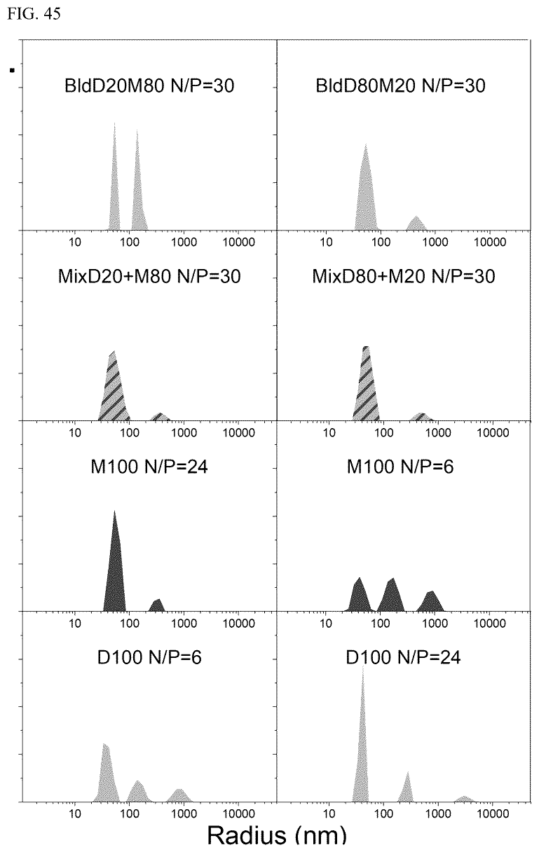

Due to the ability of M to modulate D's performance in the blended and mixed systems, these formulations were studied at different ratios of D and M to enable optimization of performance. The corresponding D100 and M100 homomicelles at N/P formulation ratios that correlate to the amount of D and M present in the mixed and blended systems were also studied. There was little difference in the micelleplex size and dispersity for the blended diblock systems and the micelle mixed systems (FIG. 45). There were some aggregates found in the non-mixed micelleplex systems (M100 N/P=6 and D100 N/P=6 and 24) that were not observed if the system was mixed or blended. These aggregates are likely caused by multiple micelleplexes that are ionically aggregated together by the ASOs. These aggregates are more common in lower N/Ps as there are more ASOs to cause ionic crosslinking of micelles. Overall, the micelleplex size is not significantly different between all the tested systems and therefore is likely not be the cause of any differences in transfection.

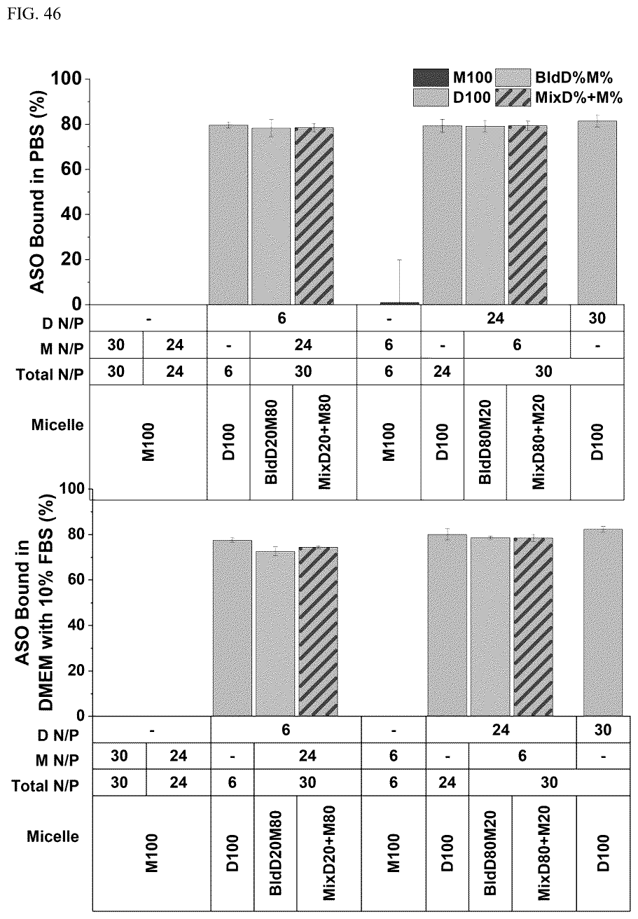

Binding was studied using the OLIGGREEN assay to observe if changing the ratio of D and M would affect binding in either PBS (the media used for micelleplex formulation) or after addition of DMEM with 10% FBS (the media used for transfection) (see Example 2, Binding Study). It was hypothesized that because all N/Ps were well above 1 that formulations including D (D100, BldD20M80 MixD20+M80, BldD80M20, and MixD20+M80) would bind similarly (as previously observed when studying only the 50:50 mixtures, FIG. 40). All formulations with D had approximately 70-80% binding with no major differences in both PBS or DMEM, indicating that the amount of ASO bound to these micelles is consistent and will not be a factor in transfection performance (FIG. 46). Additionally, this indicates that when DMEM and FBS are added there is no significant unpackaging of these systems, noting that these delivery systems are potentially useful in vivo. While unpackaging of our ASOs in this system cannot be ruled out, under the 10% FBS conditions, there appears to not be enough protein to release the ASO from our micelles in the transfection media.

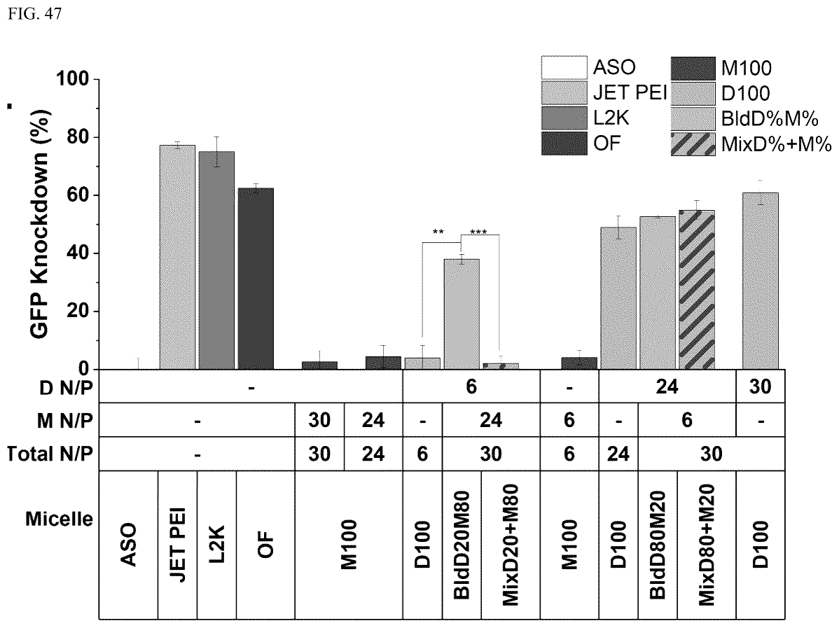

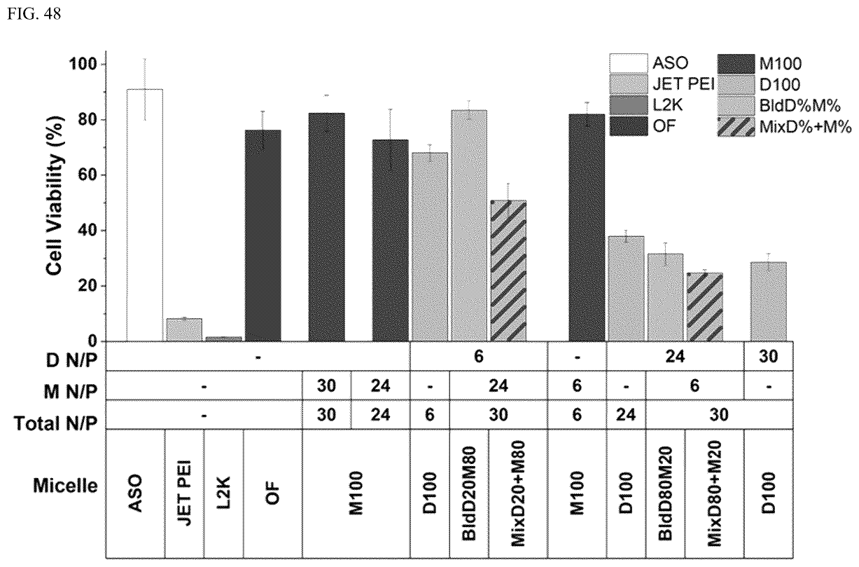

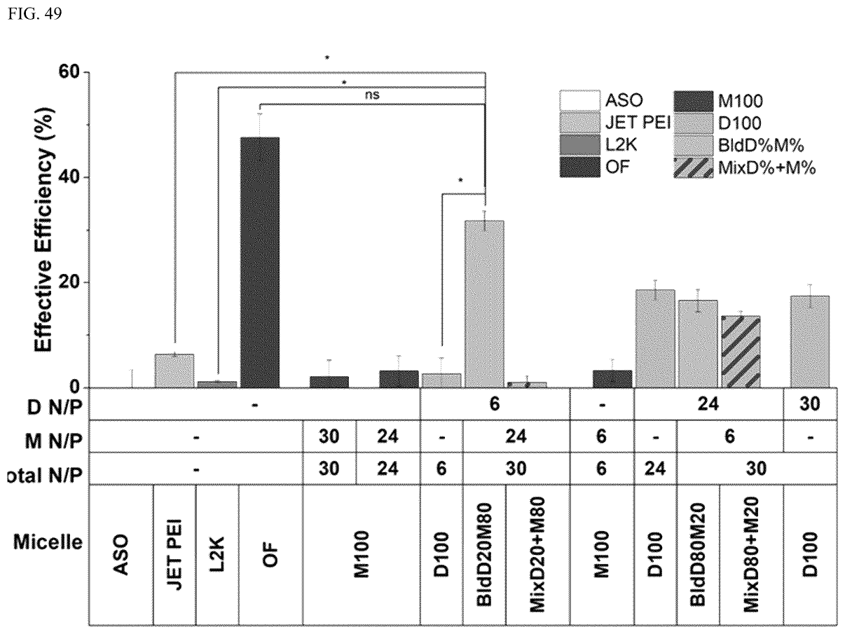

Transfection of the D and M mixed and blended systems with ASO payload was further examined to determine optimal ratios of D:M. It was hypothesized that increasing the amount of M in the system (whether as a mixed micelle or blended diblock) would improve transfection efficacy because there would be a greater change in pKa of the D moieties when more hydrophobic M is added. Additionally, the hydrophobic M moiety itself could improve delivery as increasing hydrophobicity often improves nucleic acid delivery through a variety of different mechanisms including modulation of nucleic acid binding/release, internalization, membrane-interactions, etc. Furthermore, hydrophobicity can cause differences in cooperative protonation/deprotonation, a factor in delivery of oligonucleotide cargos. One of the mixed systems with higher M incorporation, BldD20M80 N/P=30, had a much higher GFP knockdown compared to the D100 micelle at the corresponding N/P (D100 N/P=6) (FIG. 47). BldD20M80 had low toxicity (FIG. 48) as well as high knockdown hence it had the highest effective efficiency of all of the systems studied (FIG. 49). BldD20M80 had higher effective efficiency than the commercial controls L2K and JetPEI and had no statistically significant difference to the commercial control OLIGOFECTAMINE. The mixed micelle at this ratio (MixD20+M80) did not perform as well indicating that co-formulating these polymers in the same micelle is impacts performance.

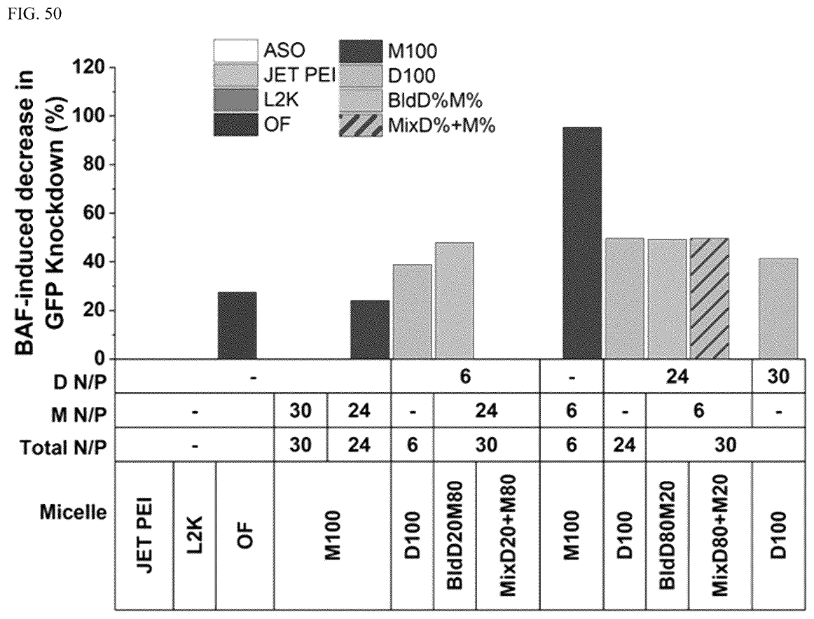

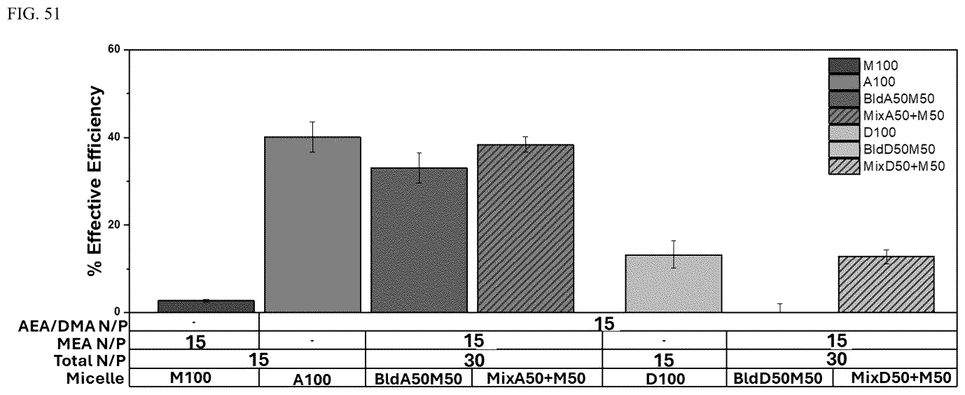

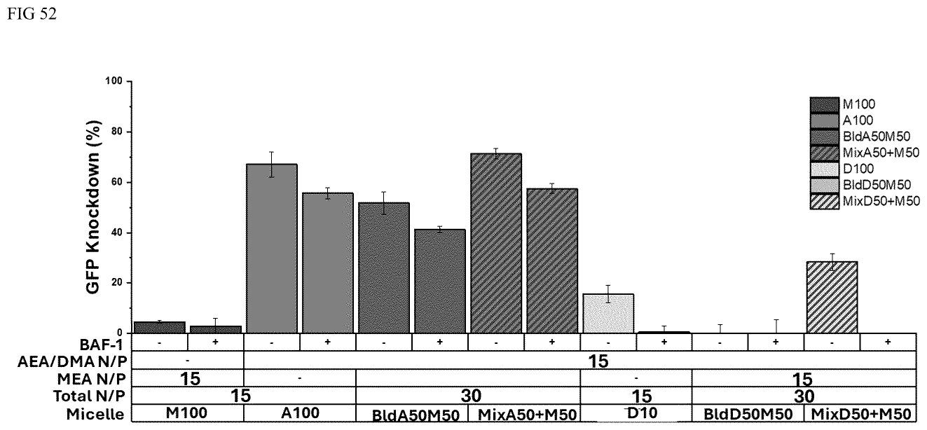

Transfection with and without Baf-A1 was examined to understand if this trend was due to changes in endosomal escape through the proton sponge hypothesis since Baf-A1 blocks proton pumps in the endosome. BldD20M80 had the most decrease in knockdown when Baf-A1 was added versus when it wasn't compared to MixD20+M80 and D100, indicating that BldD20M80 is using the proton sponge effect more than MixD20+M80 and D100, possibly explaining its high performance (FIG. 50; see also FIG. 51 and FIG. 52). At high D ratios, there are no significant differences between D100, BldD80M20, and MixD80+M20. Overall, mixing of D and M can cause modulations in endosomal escape through the proton sponge effect. These modulations may occur due to M changing the pKa of the D cationic moiety and hence increasing its proton sponge effect rather than M acting as a proton sponge itself in our original hypothesis. However, current experimental limitations make pKa measurements of micelles and micelleplexes difficult due to the high concentrations of micelles needed (too high to form monomodal micelles) to measure pKa. Mixing micelles and blending diblocks can improve performance by changing the amount of endosomal escape through the proton sponge effect.

Mixing micelles and blending diblocks systems can lead to improved transfection efficiency over homomicelle systems. This approach provides an easy and modular formulation method for optimized delivery into the cells. M could improve transfection efficiency when mixed or blended with D, but did not improve efficacy when mixed or blended with A. This may be due to the static charge density of the chains containing A within the cellular and endosomal pH range. Micelles including D were much more affected by addition of Baf-A1 indicating said formulations utilize a proton sponge mechanism of endosomal escape (compared to micelle formulations including A that were much lesser affected). M is able to increase endosomal escape using the proton sponge when D is in the system. However, A is not as reliant on endosomal escape via proton sponge effect. Therefore, no improvements in transfection were observed when M is mixed or blended with A. Additionally, the ratios of D and M were tuned within blended diblock and mixed micelle formulations. It was found that a ratio of 80:20 M:D as a blended diblock (BldD20M80) yielded optimal ASO transfection in this system.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

As used herein, “have,” “has,” “having,” “include,” “includes,” “including,” “comprise,” “comprises,” “comprising” or the like are used in their open-ended inclusive sense, and generally mean “include, but not limited to,” “includes, but not limited to,” or “including, but not limited to.” Further, wherever embodiments are described herein with the language “have,” “has,” “having,” “include,” “includes,” “including,” “comprise,” “comprises,” “comprising” and the like, otherwise analogous embodiments described in terms of “consisting of” and/or “consisting essentially of” are also provided. The term “consisting of” means including, and limited to, that which follows the phrase “consisting of.” That is, “consisting of” indicates that the listed elements are required or mandatory, and that no other elements may be present. The term “consisting essentially of” indicates that any elements listed after the phrase are included, and that other elements than those listed may be included provided that those elements do not interfere with or contribute to the activity or action specified in the disclosure for the listed elements.

In the preceding description, particular embodiments may be described in isolation for clarity. Reference throughout this specification to “one embodiment,” “an embodiment,” “certain embodiments,” “one or more embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

As used herein, the word “exemplary” means to serve as an illustrative example and should not be construed as preferred or advantageous over other embodiments.

In several places throughout the above description, guidance is provided through lists of examples, which examples can be used in various combinations. In each instance, the recited list serves only as a representative group and should not be interpreted as an exclusive list.

For any method disclosed herein that includes discrete steps, the steps may be performed in any feasible order. And, as appropriate, any combination of two or more steps may be performed simultaneously.

As used herein, the terms “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

As used herein, the terms “formed from” and “polymerized from” are open ended, unless expressly stated, and may include other components that may not be expressly described relative to the subject that is formed from or polymerized from the stated components. For example, a polymer formed from or polymerized from a quinoline-containing monomer may include capping groups or other groups not expressly mentioned.

### EXAMPLES

The present invention is illustrated by the following examples. It is to be understood that the particular examples, materials, amounts, and procedures are to be interpreted broadly in accordance with the scope and spirit of the invention as set forth herein.

### Example 1

### Materials:

N-Boc-ethylenediamine; N,N-dimethylethylenediamine; N,N-diethylethylenediamine, (2-aminoethyl)trimethylammonium chloride hydrochloride; 4-(2-aminoethyl)morpholine; and trifluoroacetic acid were purchased from Sigma Aldrich (St. Louis, MO). Human embryonic kidney cells that express destabilized green fluorescent protein (deGFP HEK cells) were procured. deGFP knockdown (+T+T+GCCGGTGGTGCAGAT+A+A+A (SEQ ID NO: 1)) and deGFP knockdown mismatched sequence ASOs (+G+G+AGTACACTATATCGG+T+G+G (SEQ ID NO: 2)) were purchased from Integrated DNA Technologies (Coralville, IA). ASO modifications included phosphorothioated backbones. Additionally, the +indicates a locked nucleic acid. Dulbecco's Modified Eagle Medium (DMEM), high glucose, GLUTAMAX FBS, trypsin-EDTA, penicillin-streptomycin, PBS, optimum, OLIGOFECTAMINE, LIPOFECTAMINE 2000, QUANT-IT OliGreen ssDNA Reagent, and calcein violet stain were from Invitrogen Life Technologies ThermoFisher Scientific (Carlsbad, CA). JetPEI was purchased through Polyplus (Illkirch-Graffenstaden, France). CCK-8 kit was purchased from Dojindo Molecular Technologies (Rockville, MD).

### Polymer Synthesis:

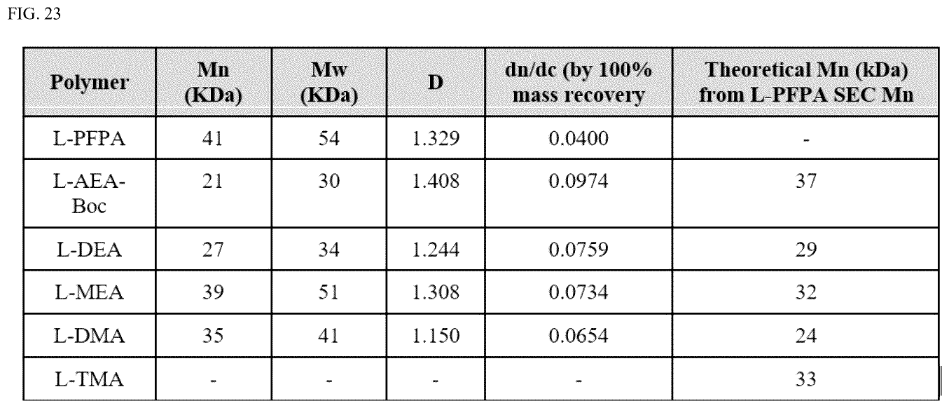

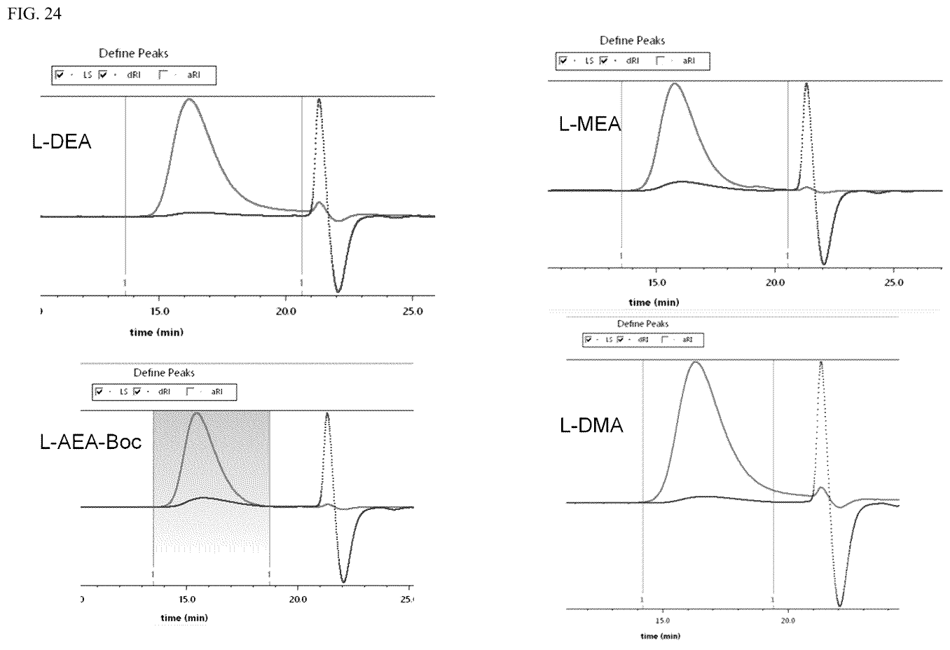

The synthesis of the cationic linear and diblock polymers from poly(pentafluorophenyl acrylate) (PPFPA) followed the procedure outlined in Santa Chalarca, C. F.; Dalal, R. J.; Chapa, A.; Hanson, M. G.; Reineke, T. M. Cation Bulk and PKa Modulate Diblock Polymer Micelle Binding to PDNA. ACS Macro Lett. 2022, 588-594. doi.org/10.1021/acsmacrolett.2c00015.). Briefly, the diblock or homopolymer with pendant PFPA was mixed with the ethylamine diamine-functionalized derivative of each cationic moiety and 2-hydroxyethyl acrylate (HEA) in 1,4-dioxane, or N,N-Dimethyl formamide (DMF) for TMA and MEA. The solution was stirred under nitrogen for 18 hours at 40° C. Dialysis in saline, followed by water, was used for purification. FIG. 23 shows the Mn, Mw, dispersity (D), dn/dc, and theoretical Mn for various polymers. FIG. 24 shows SEC traces of various purified linear polymers.

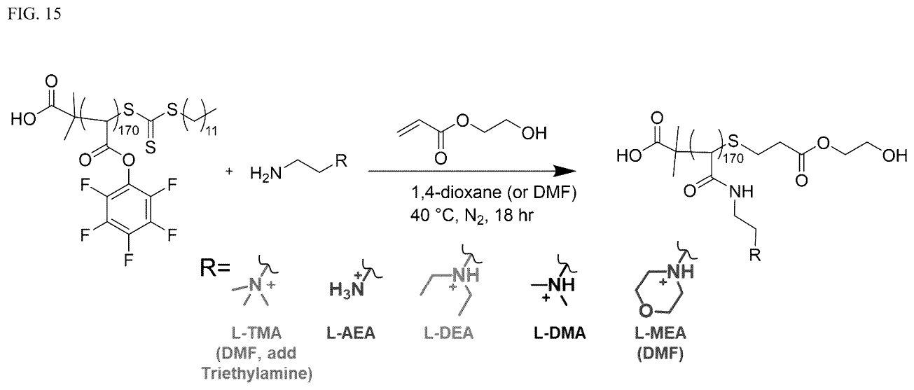

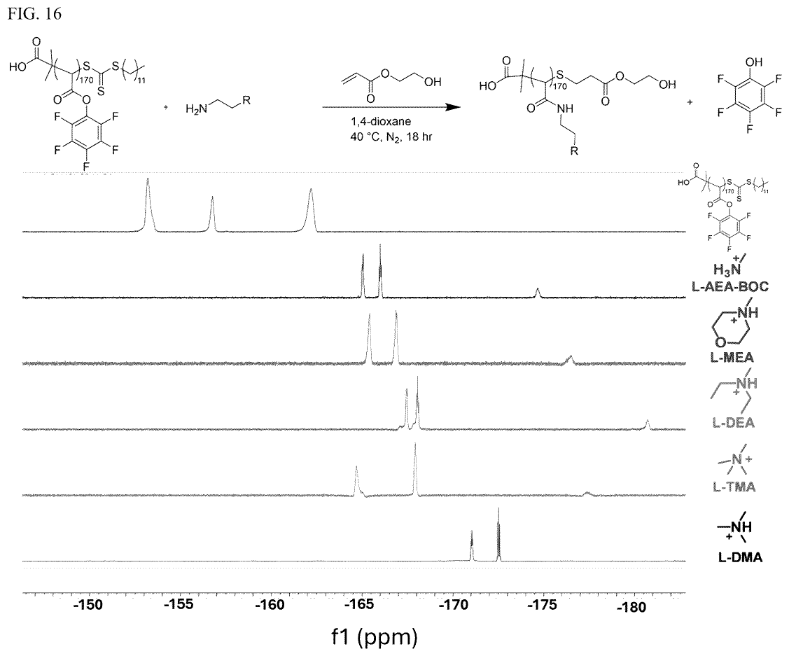

PPFPA was synthesized as described in Santa Chalarca, C. F.; Dalal, R. J.; Chapa, A.; Hanson, M. G.; Reineke, T. M. Cation Bulk and PKa Modulate Diblock Polymer Micelle Binding to PDNA. ACS Macro Lett. 2022, 588-594. doi.org/10.1021/acsmacrolett.2c00015. Aminolysis and Michael addition of the PPFPA yielded the five different linear polymers following the general procedure described in FIG. 15. Aminolysis was monitored by ¹⁹F NMR (FIG. 16).

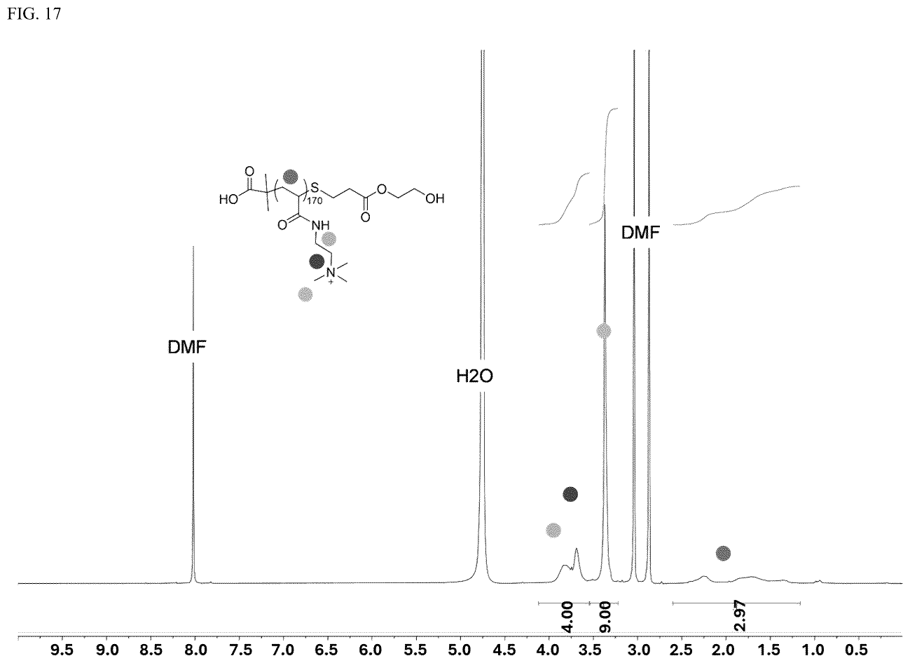

L-TMA Synthesis: To a 4 mL scintillation vial, the PFPA polymer (1 eq. per PFPA repeat unit, Mn=40K, 71.4 mg) dissolved in anhydrous DMF (2.1 mL) was added. Next, HEA (0.15 eq. per PFPA repeat unit, 25 eq. per end group, 5.1 μL) was run through a basic alumina plug column and added. Triethylamine (2 eq. per PFPA repeat unit, 83.6 μL) was added at the same time as (2-aminoethyl)trimethylammonium chloride hydrochloride (2 eq. per PFPA repeat unit, 105 mg) was added. The vial was sealed with a septum, put under N2 and heated at 40° C. for 18 hours. Next an ¹⁹F NMR was taken (FIG. 17). After confirming disappearance of PFPA polymer fluorine peaks, 15 mL of milliQ water was added to the solution which was then transferred to a dialysis membrane (Regenerated Cellulose (RC) Molecular Weight Cutoff (MWCO) 12-15 kDa). This was dialyzed against 100 mM NaCl for 2 changes/2 days and then MilliQ water for 3 changes/3 days. The solution was then freeze dried. Next, the product was dissolved in MilliQ water and run through a GHP 0.2 μm filter since there was some sort of greasy impurity in the NMR. After lyophilization, this yielded the L-TMA polymer.

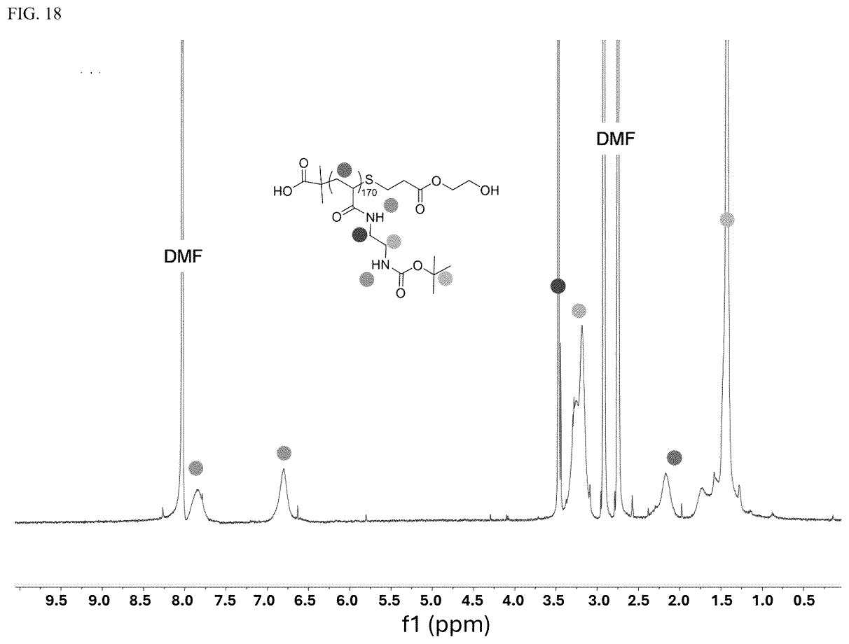

L-AEA-Boc Synthesis: To a 20 mL scintillation vial, the PFPA polymer (1 eq. per PFPA repeat unit, Mn=40K, 119.0 mg) dissolved in anhydrous 1,4 dioxane (3.6 mL) was added. Next, HEA (0.15 eq. per PFPA repeat unit, 25 eq. per end group, 5.1 μL) was run through a basic alumina plug column and added. Next the N-Boc-ethylenediamine (2 eq. per PFPA repeat unit, 158 μL) was added. The vial was sealed with a septum, put under N2 and heated at 40° C. for 18 hours. Next an ¹⁹F NMR was taken (FIG. 18). After confirming disappearance of PFPA polymer fluorine peaks, 15 mL of milliQ water was added to the solution which was then transferred to a dialysis membrane (RC MWCO 12-15 kDa). This was dialyzed against 100 mM NaCl for 2 changes/2 days and then MilliQ water for 3 changes/3 days. The solution was then freeze dried yielding the L-AEA-Boc polymer.

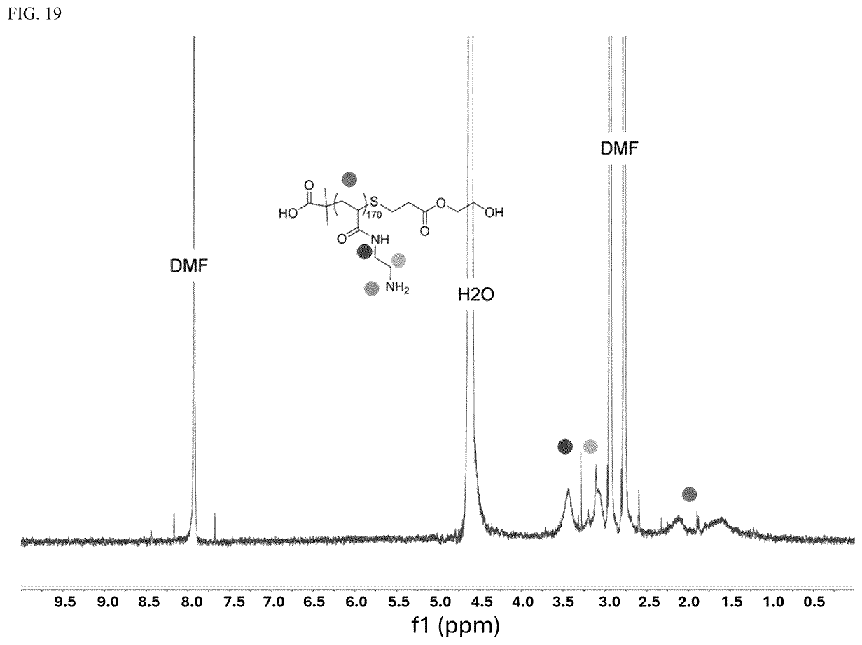

L-AEA Synthesis: To a 4 mL scintillation vial, the L-AEA-Boc polymer (10.9 mg) and trifluoroacetic acid (TFA) (0.4 mL) were added. This was stirred at room temperature for 2 hours. Next, the TFA was evaporated off using a rotary evaporator. Next, 5 mL of milliQ water was added to the resulting L-AEA polymer which was then transferred to a dialysis membrane (RC MWCO 12-15 kDa). This was dialyzed against 100 mM NaCl for 2 changes/2 days and then MilliQ water for 3 changes/3 days. The solution was then freeze dried yielding the L-AEA polymer. The ¹H NMR of L-AEA is shown in FIG. 19.

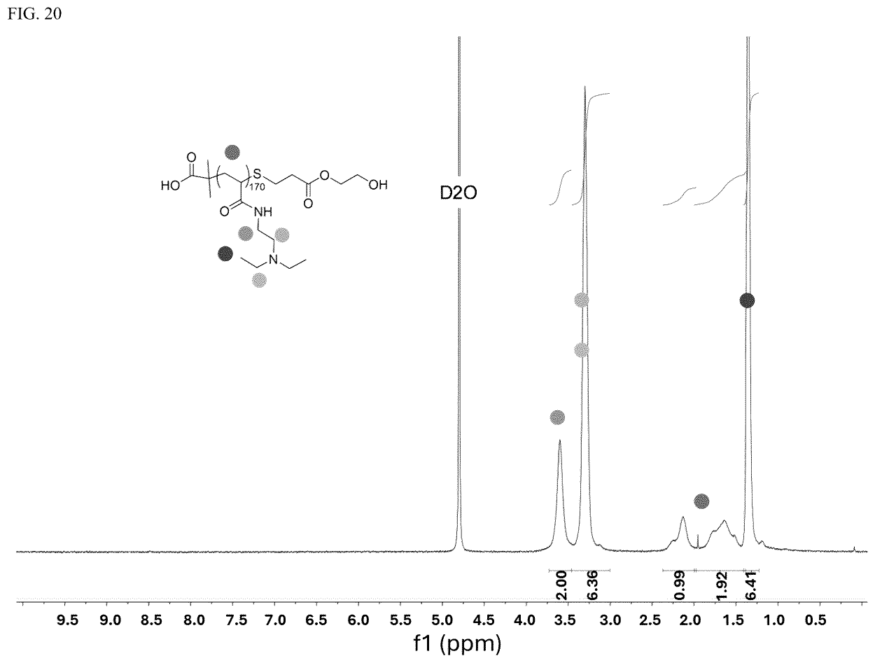

L-DEA Synthesis: To a 20 mL scintillation vial, the PFPA polymer (1 eq. per PFPA repeat unit, Mn=40K, 119.0 mg) dissolved in anhydrous 1,4 dioxane (3.6 mL) was added. Next, HEA (0.15 eq. per PFPA repeat unit, 25 eq. per end group, 5.1 μL) was run through a basic alumina plug column and added. Next the N,N-diethylenediamine (2 eq. per PFPA repeat unit, 141.7 μL) was added. The vial was sealed with a septum, put under N2 and heated at 40° C. for 18 hours. Next an ¹⁹F NMR was taken (FIG. 20). After confirming disappearance of PFPA polymer fluorine peaks, 15 mL of milliQ water was added to the solution which was then transferred to a dialysis membrane (RC MWCO 12-15 kDa). This was dialyzed against 100 mM NaCl for 2 changes/2 days and then MilliQ water for 3 changes/3 days. The solution was then freeze dried yielding the L-DEA polymer.

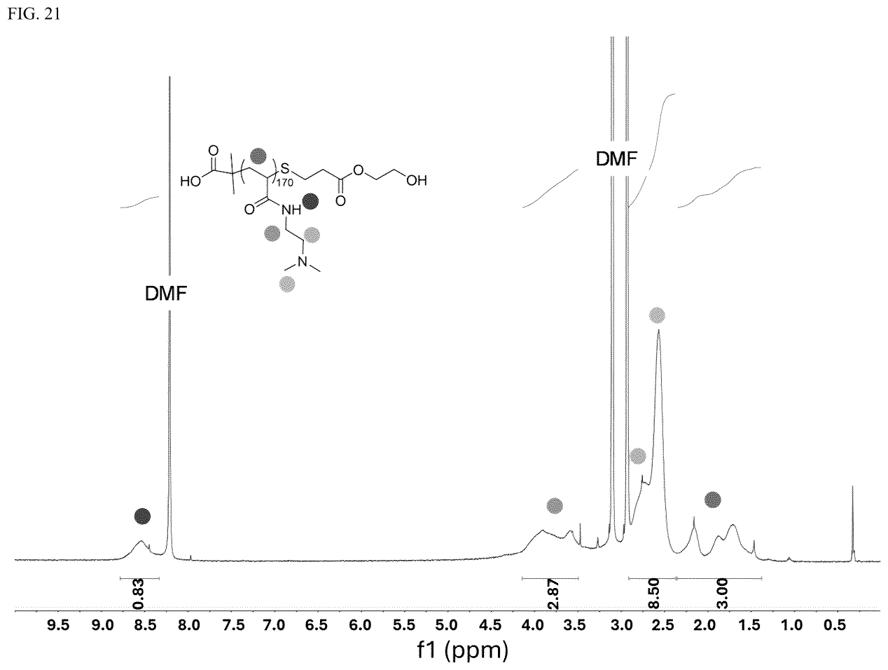

L-DMA Synthesis: To a 4 mL scintillation vial, the PFPA polymer (1 eq. per PFPA repeat unit, Mn=40K, 23.81 mg) was dissolved in anhydrous 1,4 dioxane (0.71 mL) was added. Next, HEA (0.15 eq. per PFPA repeat unit, 25 eq. per end group, 5.1 μL) was run through a basic alumina plug column and added. Next the N,N-dimethylenediamine (2 eq. per PFPA repeat unit, 21.8 μL) was added. The vial was sealed with a septum, put under N2 and heated at 40° C. for 18 hours. Next an ¹⁹F NMR was taken (FIG. 21). After confirming disappearance of PFPA polymer fluorine peaks, 15 mL of milliQ water was added to the solution which was then transferred to a dialysis membrane (RC MWCO 12-15 kDa). This was dialyzed against 100 mM NaCl for 2 changes/2 days and then MilliQ water for 3 changes/3 days. The solution was then freeze dried yielding the L-DMA polymer.

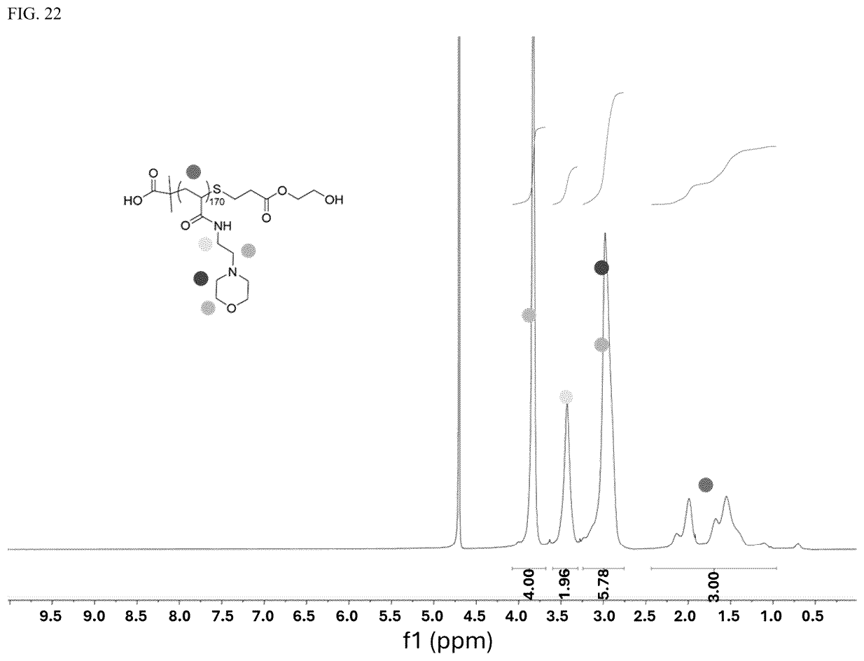

L-MEA Synthesis: To a 20 mL scintillation vial, the PFPA polymer (1 eq. per PFPA repeat unit, Mn=40K, 119.0 mg) dissolved in anhydrous DMF (3.6 mL) was added. Next, HEA (0.15 eq. per PFPA repeat unit, 25 eq. per end group, 5.1 μL) was run through a basic alumina plug column and added. Next the 4-(2-aminoethyl)morpholine (2 eq. per PFPA repeat unit, 131.2 μL) was added. The vial was sealed with a septum, put under N2 and heated at 40° C. for 18 hours. Next an ¹⁹F NMR was taken (FIG. 22). After confirming disappearance of PFPA polymer fluorine peaks, 15 mL of milliQ water was added to the solution which was then transferred to a dialysis membrane (RC MWCO 12-15 kDa). This was dialyzed against 100 mM NaCl for 2 changes/2 days and then MilliQ water for 3 changes/3 days. The solution was then freeze dried yielding the L-DEA polymer.

### Micelle Formulation:

The formulation of the cationic micelles followed the procedure outlined in Santa Chalarca, C. F.; Dalal, R. J.; Chapa, A.; Hanson, M. G.; Reineke, T. M. Cation Bulk and PKa Modulate Diblock Polymer Micelle Binding to PDNA. ACS Macro Lett. 2022, 588-594. doi.org/10.1021/acsmacrolett.2c00015.). Briefly, diblock polymers were dissolved in MOPS (10 mM, ionic strength=100 mM, pH=7.0) at a 1 mg/mL concentration and stirred for 7 days. Next, the solutions were ultrasonicated for 2 hours, annealed at 40° C. overnight, and ultrasonicated again for 2 hours. Micelle size and dispersity were determined via dynamic light scattering (DLS) on a Zetasizer Nano ZS (Malvern Instruments Limited, Worcestershire, UK, Model: ZEN3600).

### Transfection Study:

Complexes were formed by adding 50 μL of micelle or linear polymer solution including JETPEI at the corresponding N/P concentration in PBS to 50 μL of ASO (0.5 μg/well, 50 μL of ASO stock solution per well) in PBS. For OLIGFECTAMINE, the ASO was added to OLIGFECTAMINE and prepared in Optimem as outlined in the recommended procedure for this product. This was pipette mixed 3 times and then allowed to sit and complex for 30 minutes.

For the adhered cell condition, 200 μL of DMEM with 10% FBS containing 1% penicillin-streptomycin was added to the above solutions, and pipette mixed 3 times. This was added to adhered cells (plated 24 hours before at 50,000 cells per well in a 24 well plate). LIPOFECTAMINE 2000 was formulated with ASO payloads according to the commercial procedure, but scaled down to accommodate 0.5 μg of ASO/well.

For the lifted cell addition procedure, 200 μL of DMEM with 10% FBS and 1% penicillin-streptomycin was formulated with 50,000 cells and the above solutions were added and pipette mixed 3 times. This solution was added to an empty 24 well plate. Lipofectamine 2000 formulated with the commercial procedure with ASO (scaled down to 0.5 μg of ASO/well) was added to 200 μL of DMEM with 10% FBS and 1% penicillin-streptomycin with 50,000 cells that had just been added to the plate rather than adhered cells. For untreated cells, only 200 μL of DMEM+10% FBS and 1% penicillin-streptomycin and cells to each well.

24 hours after transfection, the media was replaced with 1 mL of DMEM containing 10% FBS and 1% penicillin-streptomycin. 72 hours after transfection, a CCK-8 assay was run following the commercial procedure to measure viability followed by flow cytometry to measure deGFP knockdown in live cells.

Knockdown was measured through flow cytometry by gating for cells and single cells using the forward and side scattering detectors. Cells were then gated by live cells using a calcein violet stain. The mean fluorescence of GFP in the live cells was used to analyze the GFP knockdown. The decrease in GFP fluorescence compared to untreated cells was normalized to untreated cells to get a percent knockdown value with 100% knockdown meaning no GFP fluorescence was observed.

### Cy3 ASO Internalization Study:

Cells were transfected using the same methods outlined under the transfection study, however a 5′ Cy3 labeled ASO was used. The same molarity of Cy3 ASO as ASO was used therefore, 0.547 μg Cy3 ASO per well was used to account for the added molecular weight of the Cy3 in the Cy3 ASO. 24 hours after transfection, the cells were trypsinized, washed with PBS, incubated in cell scrub for 10 min, and analyzed via flow cytometry to measure Cy3 fluorescence of live cells using a similar method described in the Transfection Study section.

### Binding Studies:

The complexes were formed by adding 50 μL micelle or linear polymer diluted at the corresponding N/P concentration to 50 μL ASO (1 μg ASO per well and 50 μL ASO stock per well). These were allowed to complex for 30 min. Next, 100 μL of QUANT-IT OLIGREEN (200x diluted from stock) was added and allowed to complex for 5 min. After, 66.6 μL of the complex QUANT-IT OLIGREEN mixture was transferred to another well and 133.3 μL of either PBS or FLUOROBRITE with 10% FBS. QUANT-IT OLIGREEN fluorescence intensity was measured on a Synergy H1 Hybrid Reader with excitation at 485 nm and emission at 528 nm (BioTek, Winooski, VT).

### Dynamic Light Scattering (DLS):

Working solutions of linear polymers, micelles, and ASO were filtered with a 0.2 μm hydrophilic polypropylene (GHP) filter. Complexes were formed by adding 33.3 μL micelle/polymer (at corresponding N/P ratio) to 33.3 μL ASO (0.6 μg/uL ASO). These were allowed to complex for 30 min. Then, 133.3 μL unfiltered DMEM with 10% FBS and 1% penicillin-streptomycin was added to each and let complex for 1 min. Note, there was not a difference in the DLS traces of filtered and unfiltered DMEM with 10% FBS and 1% penicillin-streptomycin, so unfiltered media was used to preserve FBS contents and avoid changes in concentration and/or shear-related denaturing. Complexes were then analyzed via DLS on a Wyatt DynaPro III instrument (Wyatt Technology, Santa Barbara, CA).

### Settling Study:

ASO concentration in the supernatant was compared after centrifugation to measure the amount of ASO that was in an aggregated state and “settled down”. To measure this, complexes were formed by adding linear polymers and micelles (100 μL) to Cy3-ASO (100 μL) (0.5 μg Cy3 ASO per centrifuged tube) at each designated N/P ratio. This was allowed to complex for 30 min. Next, 400 μL of FLUOROBRITE with 10% FBS was added to each complex solution. This was allowed to aggregate for 30 min. The vials were then centrifuged (15,700 rcf, 2 min). The supernatant was sampled (100 μL) and Cy3 fluorescence was measured on a Synergy H1 Hybrid Reader (Excitation: 488, Emission: 570). To normalize with respect to the ‘non-settled’ control, the vials were then vortexed for 2 min. 100 μL was sampled again from the solution and Cy3 fluorescence was measured on the plate reader. The relative amount of ASO in the supernatant was calculated as the ‘centrifuged fluorescence’ divided by the ‘vortexed fluorescence’ of each vial.

### Cell Culture:

deGFP HEK cells were maintained in Dulbecco's Modified Eagle Medium (DMEM), high glucose, GLUTAMAX Supplement (with high glucose, GLUTAMAX, and phenol Red, without sodium pyruvate) supplemented with 10% fetal bovine serum (FBS) and 1% penicillin streptomycin. Cells were kept in a 5% CO2 incubator at 37° C.

### Example 2

### Materials:

PFPA-nBa diblock copolymers were synthesized via our previously described in Example 1; N,N-dimethylethylenediamine, N-Boc-ethylenediamine, 4-(2-aminoethyl)morpholine; and trifluoroacetic acid were purchased from Sigma Aldrich (St. Louis, MO). GFP knockdown ASO (+T+T+GCCGGTGGTGCAGAT+A+A+A(SEQ ID NO: 1)) and GFP knockdown MM ASO (+G+G+AGTACACTATATCGG+T+G+G(SEQ ID NO: 2)) (+indicates a locked nucleic acid) were purchased from Integrated DNA Technologies (Coralville, IA). All ASOs had phosphorothioated backbones. Modified HEK cells that continually produce destabilized green fluorescent protein (deGFP HEK cells) were. CCK-8 kit was purchased from Dojindo Molecular Technologies (Rockville, MD). Dulbecco's Modified Eagle Medium (DMEM) high glucose GLUTAMAX, Fetal bovine serum, penicillin-streptomycin, trypsin-EDTA, phosphate-buffered saline, LIPOFECTAMINE 2000, OLIGOFECTAMINE, QUANT-IT OLIGREEN ssDNA Reagent, and calcein violet stain were from Invitrogen Life Technologies ThermoFisher Scientific (Carlsbad, CA). JETPEI was purchased through Polyplus (Illkirch-Graffenstaden, France). Bafilomycin-A1 was purchased from Cayman Chemical Company (Ann Arbor, MI).

### Diblock Synthesis:

The synthetic steps followed a similar procedure to Santa Chalarca, C. F.; Dalal, R. J.; Chapa, A.; Hanson, M. G.; Reineke, T. M. Cation Bulk and PKa Modulate Diblock Polymer Micelle Binding to PDNA. ACS Macro Lett. 2022, 588-594. doi.org/10.1021/acsmacrolett.2c00015. A PFPA-nBA diblock polymer was synthesized via RAFT chain extension of a PnBA macro-CTA. The PFPA-nBA diblock polymer was modified using either N-Boc-ethylenediamine(A-Boc protected), N,N-dimethylethylenediamine (D), or 4-(2-aminoethyl)morpholine (M) for aminolysis and HEA for thiocarboate cleavage and subsequent Michael addition in either 1,2-dioxane or DMF by stirring at 40° C. for 18 hours. The resulting solution was purified via dialysis in saline solution followed by Millipore water to yield the diblock polymers A-Boc protected, D, and M after freeze drying. The A-Boc protected polymer was deprotected by mixing with TFA for 2 hours followed by dialysis in saline solution followed by water to yield the A polymer after freeze drying.

### Homomicelle Formation:

The micelle formulation steps followed the general procedure outlined in Santa Chalarca, C. F.; Dalal, R. J.; Chapa, A.; Hanson, M. G.; Reineke, T. M. Cation Bulk and PKa Modulate Diblock Polymer Micelle Binding to PDNA. ACS Macro Lett. 2022, 588-594. doi.org/10.1021/acsmacrolett.2c00015. Polymers were directly dissolved in MOPS (20 mM, pH=7.0 100 ionic strength adjusted with NaCl) at 1 mg/mL in a 20 mL glass vial. This was stirred at room temperature for 7 days. After 7 days, the solutions were ultrasonicated for 2 hours.

### Dynamic Light Scattering (DLS) for Micelles:

Micelle size and dispersity were determined via DLS at 1 mg/mL concentrations on a Zetasizer Nano ZS (Malvern Instruments Limited, Worcestershire, UK, Model: ZEN3600).

### Dynamic Light Scattering (DLS) for Micelleplexes:

The micelleplexes were formed mimicking the transfection procedure by adding 50 μL micelle solution diluted at the corresponding N/P concentration to 50 μL ASO solution (0.25 μg ASO per well and 50 μL ASO stock per well). These were both diluted in PBS. These were allowed to complex for 30 min. Complexes were then analyzed via DLS on a Wyatt DynaPro III instrument (Wyatt Technology, Santa Barbara, CA). For studies in DMEM with 10% FBS, 200 μL of DMEM with 10% FBS and 1% penstrep was added. Complexes were then analyzed again via DLS on a Wyatt DynaPro III instrument.

### Binding Studies:

The micelleplexes were formed mimicking the transfection process. 50 μL micelle solution diluted at the corresponding N/P concentration was added to 50 μL ASO solution (0.25 μg ASO per well and 50 μL ASO stock per well). These were all diluted in PBS. These were allowed to complex for 30 min. Next, 0.5 μL of QUANT-IT OLIGREEN stock was added and allowed to complex for 5 min. Fluorescence intensity of the QUANT-IT OLIGREEN was measured on a Synergy H1 Hybrid Reader (excitation=485 nm, emission=528 nm) (BioTek, Winooski, VT). Next, if studied in DMEM with 10% FBS, 200 μL of DMEM with 10% FBS and 1% penstrep was added followed by chloroacetate buffer (if studied and at the concentration desired). Fluorescence intensity of the QUANT-IT OLIGREEN was measured again on the Synergy H1 Hybrid Reader (excitation=485 nm, emission=528 nm.

### Blended Diblock Formation:

The two different diblocks were mixed in the powder form. This mixture was then directly dissolved in MOPS (20 mM, pH=7.0, 100 ionic strength adjusted with NaCl) at 1 mg/mL in a 20 mL glass vial. This was stirred at room temperature for 7 days. After 7 days, the solutions were ultrasonicated for 2 hours. DLS was used to analyze the size and dispersity of the micelles using a Zetasizer Nano ZS (Malvern Instruments Limited, Worcestershire, UK, Model: ZEN3600).

### Transfection:

In a centrifuge tube, 50 μL of micelle solutions (concentration depended on N/P studied) made from either pre-formed homomicelles or blended diblock micelles or by mixing two solutions of homomicelles to form a mixed micelle was added to 50 μL of ASO solutions (5 ug/mL ASO so that each well contained 0.25 ug ASO (note: Example 1 studies were done at 0.5 ug ASO per well 22), pipette mixed, and then allowed to complex for 30 min to form the micelleplex solutions. Next, 200 μL of DMEM with 10% FBS and 1% penicillin-streptomycin containing 50,000 deGFP HEK cells was added to the 100 μL of micelleplex solution, chloroacetate (at designated concentration if studied) pipette mixed and added to the well in a 24 well plate. LIPOFECTMINE 2000 was formulated similarly to the commercial protocol however it was scaled down to 0.25 ug ASO per well. Additionally, 200 μL of DMEM with 10% FBS and 1% penicillin-streptomycin with 50,000 cells was added to the lipofectamine-ASO solution and this was mixed and added to the plate instead of adding it to adhered cells. For untreated cells, 200 μL of DMEM+10% FBS and 1% penicillin-streptomycin with 50,000 cells was added to 100 uL PBS (instead of micelleplex solution), mixed and added to each well. These cell and micelleplex mixtures were allowed to sit and settle for 30 minutes before being put in an 5% CO2, 37° C. incubator.

24 hours after introducing the cells to the micelleplexes, the media was changed out for 1 mL of DMEM containing 10% FBS and 1% penicillin-streptomycin. 72 hours after transfection, cells were analyzed via the CCK-8 viability assay following the commercial protocol. Additionally, the cells were analyzed via flow cytometry to measure GFP fluorescence. The forward and side scattering detectors were used to gate for cells and single cells and calcein violet staining was used to gate for live cells. GFP knockdown was analyzed using the mean fluorescence of GFP in the live, single cells. The decrease in GFP fluorescence was normalized to untreated cells to get a percent knockdown value where 100% knockdown meant no GFP fluorescence was observed.

### Bafilomycin-A1 Transfection:

Transfection conditions were identical to previous method; however, cells were preincubated with 200 nM Bafilomycin-A1 in DMEM containing 10% FBS and 1% penicillin-streptomycin for 30 minutes prior to transfection. After the pre-incubation step, cells were counted and transfected as described above.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A complex comprising:
a diblock polymer comprising:
a first block comprising an acrylate polymer; and
a second block comprising an acrylamide polymer, the acrylamide polymer comprising quaternary ammonium groups or amine cationizable groups; and
an oligonucleotide cargo.

2. The complex of claim 1, wherein the acrylamide polymer comprises amino ethyl acrylamide, dimethyl amine ethyl acrylamide, diethyl amine ethyl acrylamide, trimethyl amine ethyl acrylamide, or morpholino ethyl acrylamide.

3. The complex of claim 1, wherein the acrylate polymer comprises a n-butyl-acrylate polymer.

4. The complex of claim 1, wherein the oligonucleotide cargo comprises an antisense oligonucleotide.

5. The complex of claim 1, wherein the complex has an N/P ratio of 10 to 40.

6. The complex of claim 1, wherein the complex comprises a micelle.

7. A transfection composition comprising a plurality of the complexes of claim 1.

8. The transfection composition of claim 7, wherein the transfection composition comprises a first plurality of complexes of claim 1 and a second plurality of complexes of claim 1 wherein the first plurality of complexes and the second plurality of complexes differ by at least by the identity of the second block of the diblock polymer.

9. A transfection method comprising contacting a cell with the transfection composition of claim 7.

10. A complex comprising:
a first diblock polymer comprising:
a first block comprising an acrylate polymer; and
a second block comprising an acrylamide polymer, the acrylamide polymer comprising quaternary ammonium groups or amine cationizable groups; and
a second diblock polymer comprising:
a first block comprising an acrylate polymer; and
a second block comprising an acrylamide polymer, the acrylamide polymer comprising quaternary ammonium groups or amine cationizable groups, wherein the quaternary ammonium groups or amine cationizable groups of the second diblock polymer are different than the quaternary ammonium groups or amine cationizable groups of the first diblock polymer; and
an oligonucleotide cargo.

11. The complex of claim 10, wherein the first block of first diblock polymer, the first block of the second diblock polymer, or both comprises an n-butyl-acrylate polymer.

12. The complex of claim 10, wherein the acrylamide polymer of the first diblock polymer comprises morpholino ethyl acrylamide and wherein the acrylamide polymer of the second diblock polymer comprises amino ethyl acrylamide, dimethyl amine ethyl acrylamide, diethyl amine ethyl acrylamide, or trimethyl amine ethyl acrylamide.

13. The complex of claim 10, wherein the weight ratio or mole ratio of the first diblock polymer to the second diblock polymer is 0.8 parts or greater of the first diblock polymer for every 1 part of the second polymer.

14. The complex of claim 10, wherein the weight ratio or mole ratio of the first diblock polymer to the second diblock polymer is 0.1 to 0.3 parts of the first diblock polymer for every 1 part of the second polymer.

15. The complex of claim 10, wherein the complex comprises a micelle.

16. A transfection composition comprising a plurality of the complexes of claim 10.

17. A transfection method comprising contacting a cell with the transfection composition of claim 16.

18. A complex comprising:
a homopolymer comprising an acrylamide polymer, the acrylamide polymer comprising quaternary ammonium groups or amine cationizable groups; and
an oligonucleotide cargo, wherein the oligonucleotide cargo has a length of 1000 bases or less.

19. The complex of claim 18, wherein the complex is a polyplex.

20. The complex of claim 18, wherein the acrylamide polymer comprises amino ethyl acrylamide, dimethyl amine ethyl acrylamide, diethyl amine ethyl acrylamide, trimethyl amine ethyl acrylamide, or morpholino ethyl acrylamide.
